(19) United States
(12) Patent Application Publication
(10) Pub. No.: US 2024/0000616 A1
(43) Pub. Date: Jan. 4, 2024

(54) FLUID DRAINAGE AND DELIVERY DEVICE FOR WOUND TREATMENT

(71) Applicant: AROA BIOSURGERY LIMITED, Auckland (NZ)

(72) Inventors: Brian Roderick WARD, Pukekohe (NZ); Isaac Tristram Tane MASON, Auckland (NZ); Hamis Joshua ROSE, Auckland (NZ); Alister Todd JOWSEY, Acuckland (NZ); Michael Andrew LOVELAND, Auckland (NZ); Liam DOUGLAS, Auckland (NZ); Samuel Barry TURNER, Auckland (NZ); Brendan O'NEILL, (US); Sean James GORMAN, Auckland (NZ); Dorrin ASEFI, Auckland (NZ); Henry David CHITTOCK, Auckland (US)

(21) Appl. No.: 18/038,365

(22) PCT Filed: Nov. 24, 2021
(86) PCT No.: PCT/NZ2021/050207

§ 371 Date: May 23, 2023

Related U.S. Application Data
(60) Provisional application No. 63/281,363, filed on Nov. 19, 2021.
(60) Provisional application No. 63/217,941, filed on Jul. 2, 2021.
(60) Provisional application No. 63/117,995, filed on Nov. 24, 2020.

(51) Int. Cl. A61F 13/00; A61M 1/00

(52) U.S. Cl. CPC A61F 13/00068; A61M 1/92; A61M 1/91

(57) ABSTRACT

A device for implantation at a treatment site in the body of a patient for the removal of fluid from the treatment site. The device includes a conduit structure at least in part defining a fluid removal lumen, and a porous bioresorbable sheath surrounding a portion of the conduit structure. The conduit structure comprises a removable component configured for removal from the treatment site upon completion of treatment.

### TECHNICAL FIELD

The invention relates to a device for implanting at a wound treatment site, for the delivery of fluid to the site and for the drainage of fluid from the site. In particular, the device has a fluid supply lumen and a fluid removal lumen, and a bioresorbable sheath.

### BACKGROUND OF THE INVENTION

The drainage of fluid and the reduction of dead space from surgical or traumatic wounds is often a critical factor in the timely and effective recovery of a patient. Seromas and hematomas are pockets of serous fluid or blood that accumulate at wound sites post-surgery that can hinder recovery. In the absence of adequate drainage and dead space closure, poor healing, infection or dehiscence may lead to a requirement for additional surgery and longer hospital stays. Seromas and hematomas are common after reconstructive plastic surgery procedures, trauma, mastectomy, tumour excision, caesarean, hernia repair and open surgical procedures involving a lot of tissue elevation and separation.

While reducing dead space and providing drainage of fluid from a wound site is highly desirable in many instances, it is useful in other circumstances to be able to deliver fluid directly to a wound site to aid in the wound healing process. For example, instilling antimicrobial solutions locally to prevent infections. Similarly, instillation of local anaesthetics can aid pain management.

Prior art fluid removal devices are prone to blocking and are ineffective at preventing the formation of seroma within a soft tissue cavity. Loose tissue debris remaining at the site following surgery, such as loose connective tissue and adipose (fat) tissue, in combination with various biological factors such as fibrinogen and lysed cells tend to cause these devices to be substantially or completely block during use. Blockages reduce the ability of any device to remove fluid from a closed surgical wound and limit the effective delivery of vacuum pressure to a treatment site.

As a consequence, prior art fluid removal devices generally only apply a low level of suction (typically less than 60 mmHg of vacuum). Further, attempting to operate these devices at higher vacuums does not improve their effectiveness, it simply hastens the speed at which the devices block.

It is therefore an object of the invention to provide a fluid drainage or delivery device that addresses one or more of the abovementioned shortcomings, and/or at least to provide a useful alternative to existing devices.

In this specification where reference has been made to patent specifications, other external documents, or other sources of information, this is generally to provide a context for discussing features of the invention. Unless specifically stated otherwise, reference to such external documents or sources of information is not to be construed as an admission that such documents or such sources of information, in any jurisdiction, are prior art or form part of the common general knowledge in the art.

### SUMMARY OF THE INVENTION

In a first aspect, the present invention provides a device for implantation at a treatment site in the body of a patient for the removal of fluid from the treatment site. The device comprises a conduit structure at least in part defining a fluid removal lumen, and a porous bioresorbable sheath surrounding a portion of the conduit structure. The conduit structure comprises a removable component configured for removal from the treatment site upon completion of treatment.

In an embodiment, the device is configured to deliver a fluid to the treatment site, and wherein the conduit structure further defines a fluid supply lumen. One end of the fluid supply lumen may be in fluid communication with one end of the fluid removal lumen.

In an embodiment, the device comprises a dual lumen port for connection with one or more external components, wherein a first lumen of the port is in fluid communication with the fluid removal lumen.

In an embodiment, the bioresorbable sheath comprises a plurality of apertures positioned to enable fluid communication between the treatment site and the conduit structure, the apertures each having an area of about 1 mm² or less. For example, the apertures in the sheath may each have an area of between about 0.2 mm² to about 0.8 mm².

In an embodiment, the sheath comprises a top sheet that wraps over a top part of the conduit structure, and a bottom sheet that wraps over a bottom part of the conduit structure, wherein the top and bottom sheets are joined around the conduit structure along a side seam. The top and bottom sheets may be stitched together.

In an embodiment, the sheath forms one or more flange(s) or tab(s) extending beyond the side seam, for securing the device to tissue at the treatment site. The flanges or tabs may comprise two layers, and the layers are attached at or near an edge of the flange or tab.

In an embodiment, the apertures in the sheath are provided on upper and lower surfaces of the device.

In an embodiment, the sheath comprises an end section proximal an inlet and outlet of the device, configured to prevent or minimise the ingress of wound debris into the conduit structure. The end section of the sheath preferably does not comprise through apertures.

In an embodiment, an end of the sheath distal an inlet and outlet of the device is closed. Alternatively, an end of the sheath distal an inlet and outlet of the device may be open.

In an embodiment, the sheath comprises one or more layers of extracellular matrix (ECM) or polymeric material. The ECM may be formed from decellularised propria-submucosa of a ruminant forestomach.

In an embodiment, the fluid supply lumen of the removable conduit structure comprises a non-porous wall along at least a major part of the length of the structure.

The fluid removal lumen of the removable conduit structure may comprise a porous wall along a major part of the length of the structure.

In an embodiment, the removable conduit structure comprises a truss defining at least a major portion of the fluid removal lumen of the removable conduit structure. In an embodiment, the truss comprises two flexible elongate wall members wound such that they intersect each other periodically at a plurality of cross-over nodes. Each elongate wall member may be generally helical, and wherein the two wall members are oppositely wound. The truss may form a flexible tube having a round or oval cross-section.

In some embodiment trusses, the truss may include at least two flexible elongate bracing members, each bracing member being linked to the two elongate wall members at a plurality of the cross-over nodes. The bracing members may extend generally longitudinally along a side of the channel. The bracing truss members may be provided on opposite sides of the channel. Each bracing member may be bonded to the two elongate wall members at the respective cross-over nodes.

In an embodiment, the truss may include a securing truss member, wound to secure the truss of the fluid removal lumen to the fluid supply lumen.

In an embodiment, the removable conduit structure comprises a silicone form.

In an embodiment the fluid removal lumen has a cross-sectional area of at least 7 mm², for example a cross-sectional area of about 18 mm².

In an embodiment, the fluid removal lumen has an inlet end and an outlet end, and wherein the fluid supply lumen is configured to supply fluid to adjacent the inlet end of the fluid removal lumen.

In an embodiment, the fluid supply lumen and the fluid removal lumen are generally the same length and positioned adjacent each other.

In an embodiment, the fluid supply lumen and the fluid removal lumen are colinear. For example, the device may form a loop. In one embodiment, the loop comprises two limbs of the conduit structure with abutted ends.

In an embodiment, the device comprises a port in fluid communication with the fluid removal and/or fluid supply lumens and being connectable to a source of negative pressure or positive pressure.

The treatment site may be a region between surfaces or planes of muscle tissue, connective tissue and/or or skin tissue that have been separated during surgery or as a result of trauma, or a region within a layer of tissue.

In an embodiment, the sheath comprises a sealing end section free from apertures and having a tight fit with the underlying portion of the conduit structure. The sealing end section of the sheath extends over a portion of the conduit structure that comprises fluid impervious walls.

In an embodiment, the cross-sectional area of the sheath and the underlying conduit structure is reduced along at least a portion of the sealing section.

In an embodiment, the cross-sectional area of the sheath and the underlying conduit structure is tapered along at least a portion of the sealing section.

In a second aspect, the present invention provides a device for implantation at a treatment site in the body of a patient for the delivery of fluid to and/or removal of fluid from the treatment site. The device comprises: a conduit structure defining a fluid supply and/or removal lumen and a bioresorbable sheath surrounding a portion of the removable conduit structure. The sheath comprises a plurality of apertures sized and positioned to enable fluid communication between the treatment site and the conduit structure while preventing blockages in the device.

In an embodiment, the apertures in the sheath each have an area of between about 0.2 mm² to about 0.8 mm².

In an embodiment, the sheath comprises a sealing end section free from apertures and having a tight fit with the underlying portion of the conduit structure.

In an embodiment, the sealing end section of the sheath extends over a portion of the conduit structure that comprises fluid impervious walls.

In an embodiment, the cross-sectional area of the sheath and the underlying conduit structure is reduced along at least a portion of the sealing section.

In an embodiment, the cross-sectional area of the sheath and the underlying conduit structure is tapered along at least a portion of the sealing section.

In an embodiment, the device comprises a port in fluid communication with the lumen(s) of the conduit structure.

In an embodiment, the conduit structure comprises a removable component configured for removal from the treatment site upon completion of treatment.

The device according to the second aspect may include any one or more of the features described above in relation to the first aspect.

In a third aspect, the present invention provides a device for implantation at a treatment site in the body of a patient for the delivery of fluid to and/or removal of fluid from the treatment site; the device comprising:
a conduit structure defining a fluid supply lumen and a porous fluid removal lumen, one end of the fluid supply lumen being in fluid communication with a first end of the fluid removal lumen;
a bioresorbable sheath surrounding a portion of the removable conduit structure; and
a port in fluid communication with the fluid supply lumen and/or the fluid removal lumen(s).

In an embodiment, the device comprises a dual lumen port, with a first lumen of the port in fluid communication with the fluid supply lumen and a second lumen of the port in fluid communication with the fluid removal lumen.

A portion of the conduit structure defining the fluid supply lumen may be integrally formed with a portion of the conduit structure defining the fluid removal lumen.

In an embodiment, the fluid supply lumen and fluid removal lumen are co-axial. Alternatively, the fluid supply lumen and fluid removal lumen may be substantially parallel.

In an embodiment, the port is configured for connection with one or more external components.

In an embodiment, the sheath comprises a multiplicity of apertures to facilitate fluid transfer across the sheath, each aperture having an area of between about 0.2 mm² to about 0.8 mm².

In an embodiment, the sheath comprises a sealing end section free from apertures and having a tight fit with the underlying portion of the conduit structure.

In an embodiment, the conduit structure comprises a removable component configured for removal from the treatment site upon completion of treatment.

The device according to the third aspect may include any one or more of the features described above in relation to the first or second aspects.

In a fourth aspect, the present invention provides a system for draining fluid from a treatment site and delivering fluid to a treatment site in the body of a patient comprising:
(i) a device according to the first, second or third aspects;
(ii) a conduit releasably coupled to either a port of the device or to a fluid impermeable dressing;
(iii) a reservoir located external to the body of the patient and containing a treatment fluid, the reservoir in fluid communication with the fluid supply lumen;
(iv) a second reservoir located external to the body of the patient, the second reservoir in fluid communication with fluid removal lumen for receiving fluid from the device; and
(v) a source of pressure coupled to the conduit for delivering positive pressure or negative pressure to the device.

In an embodiment, the source of pressure is capable of delivering negative pressure to the device so that fluid is drained from the treatment site into the device and transferred through the conduit to the reservoir.

In an embodiment, the port of the device is positioned external to the patient's body.

In a fifth aspect, the present invention provides a kit of parts for forming the device as device according to the first, second or third aspects, comprising a conduit structure defining a fluid removal lumen, and a bioresorbable sheath defining a passage for receipt of the conduit structure.

In an embodiment, the bioresorbable sheath is generally tubular having two open ends.

In a sixth aspect, the present invention provides a system for treating a wound comprising: a fluid input and a fluid output for connection to a wound treatment device located at the wound. The wound treatment device may be as described above. The fluid input is adapted to be fluidly connected to an upstream side of the wound treatment device and the fluid output is adapted to be fluidly connected to a downstream side of the wound treatment device. The system further comprises an air inlet valve upstream of the fluid output; an actuator to drive the air inlet valve between an open position and a closed position; a pump downstream of the fluid input; a motor to drive the pump to provide a negative pressure to the wound treatment device; and a controller in communication with the actuator and the motor to operate the air inlet valve and the pump. The controller is configured to: i) open the air inlet valve and operate the pump to maintain a first vacuum pressure at the wound treatment device and introduce air into the wound treatment device; ii) close the air inlet valve and operate the pump to maintain a second vacuum pressure at the wound treatment device and remove air and fluid from the wound treatment device. The first vacuum pressure is less than or equal to the second vacuum pressure.

In an embodiment, the controller is configured to operate the pump to continuously maintain a negative pressure environment at the wound treatment device when the air valve is open and closed.

In an embodiment, the first and second vacuum pressures provide for effective negative pressure wound therapy.

In an embodiment, the controller is configured to repeat steps i) and ii) to cycle the air inlet valve between the open and closed positions.

In an embodiment, the controller is configured to repeat steps i) and ii) to continuously cycle the air inlet valve between the open and closed positions.

In an embodiment, the controller is configured to operate the pump when the air inlet valve is open to maintain a substantially constant first vacuum pressure.

In an embodiment, the controller is configured to operate the pump with the air inlet valve open so that a flow rate of air into the system through the air inlet valve is equal to a flow rate of the pump.

In an embodiment, the controller is configured to operate the pump when the air inlet valve is closed to maintain a substantially constant second vacuum pressure.

In an embodiment, the controller is configured to: in step (i), operate the pump with the air inlet valve open so that the system is in an equilibrium state with a zero or constant pressure differential across the treatment device.

In an embodiment, controller is configured to: in step (ii), operate the pump with the air inlet valve closed so that the system is in an equilibrium state with a zero or constant pressure differential across the treatment device.

In an embodiment, the controller is configured to operate the air inlet valve between open and closed to introduce a flow rate of air into the system that generates a bubble flow or slug flow comprising bubbles or slugs of air entrained in fluid flow from the wound treatment device.

In an embodiment, the controller is configured to operate the air inlet valve between open and closed to reduce a density of fluid at the wound to lift the fluid from the wound against gravity.

In an embodiment, the controller is configured to open and close the air inlet valve periodically.

In an embodiment, in step i) the controller is configured to open the air inlet valve for a predetermined time period. In an embodiment, in step i) the controller is configured to open the air inlet valve for at least 10 seconds.

In an embodiment, in step ii) the controller is configured to close the air inlet valve for a predetermined time period.

In an embodiment, the air inlet valve is open for at least 10% of the cycle pitch, or at least 20% of the cycle pitch, or at least 30% of the cycle pitch, or at least 40% of the cycle pitch, or at least 50% of the cycle pitch.

In an embodiment, in step i), the air inlet valve is open for a sufficient time period so that a volume of air delivered through the system is at least a substantial portion of a total volume of the system. For example in step (i), the air inlet valve may be open for a sufficient time period so that the volume of air delivered to the system is at least 50%, or at least 100% of the total volume of the system.

In an embodiment, the first vacuum pressure is about 30% to 100% of the second vacuum pressure.

In an embodiment, the first vacuum pressure is about 50 to 100 mmHg, preferably between about 80 and about 90 mmHg.

In an embodiment, the second vacuum pressure is about 100 to 150 mmHg, preferably between about 100 and about 110 mmHg.

In an embodiment, the first vacuum pressure is about 10 to 50 mmHg less than the second pressure.

In an embodiment, in step (i) the controller is configured to operate the pump to achieve a vacuum pressure threshold. In an embodiment, in step (ii) the controller is configured to operate the pump to achieve a vacuum pressure threshold.

In an embodiment, the system comprises a downstream pressure sensor located downstream of the wound treatment device and in communication with the controller. The controller may be configured to, in step i) operate the pump to achieve the vacuum pressure threshold based on a pressure sensed by the downstream pressure sensor.

In an embodiment, the system comprises an upstream pressure sensor located upstream of the wound treatment device and in communication with the controller. The controller may be configured to, in step ii), operate the pump to achieve the vacuum pressure threshold based on a pressure sensed by the upstream pressure sensor.

In an embodiment, the system comprises:
an upstream pressure sensor located upstream of the wound treatment device and in communication with the controller,
a downstream pressure sensor located downstream of the wound treatment device and in communication with the controller, and
the controller is configured to, in step i) operate the pump to achieve a first vacuum pressure threshold based on a pressure sensed by the downstream pressure sensor; and

in step ii), operate the pump to achieve a second vacuum pressure threshold based on a pressure sensed by the upstream pressure sensor.

In an embodiment, the first vacuum pressure threshold is less than or equal to the second vacuum pressure threshold.

In an embodiment, the system comprises an inlet restriction, and the upstream pressure sensor is located upstream of the inlet restriction so that the upstream pressure sensor measures ambient pressure when the air inlet valve is open.

In an embodiment, the system comprises an inlet restriction to present a predetermined pressure drop between ambient pressure and a vacuum pressure at the wound treatment device.

In an embodiment, the system comprises a filter to filter air introduced to the system, and wherein the filter is or comprises the inlet restriction.

In an embodiment, the pressure drop is approximately 20 to 130 mmHg.

In an embodiment, when the air inlet valve is open, substantially all pressure differential between ambient pressure and a pressure downstream of the wound treatment device is at the inlet restriction.

In an embodiment, the system comprises a reservoir for collecting fluid removed from the wound, and wherein the reservoir is located downstream of the pump such that fluid removed from the wound passes through the pump to the reservoir.

In an embodiment, the reservoir comprises a flexible bag.

In an embodiment, the reservoir comprises a vent to vent the reservoir to the ambient atmosphere.

In an embodiment, the system comprises a treatment fluid inlet upstream of the fluid outlet to connect a supply of treatment fluid.

In an embodiment, the system is configured so that, in step i) the introduction of treatment fluid to the wound treatment device is prevented or reduced by the introduction of air to the wound treatment device by the first vacuum pressure, and in step ii), treatment fluid is drawn to the wound treatment device by the second vacuum pressure.

In an embodiment, the system comprises:
a treatment fluid valve between the treatment fluid inlet and the fluid outlet, and
an actuator to drive the treatment fluid inlet valve between an open position and a closed position, wherein the controller is in communication with the fluid inlet valve actuator and the controller is configured to, in a fluid supply state:
   iii). open the fluid inlet valve and operate the pump to maintain a vacuum pressure at the wound treatment device and introduce treatment fluid into the wound treatment device;
   iv). close the fluid inlet valve and operate the pump to maintain a vacuum pressure at the wound treatment device and remove fluid from the wound treatment device.

In an embodiment, the controller is configured to operate the pump to continuously maintain a negative pressure environment at the wound treatment device when the fluid inlet valve is open and closed.

In an embodiment, the controller is configured to, in step (iii), operate the pump to generate a third vacuum pressure at the wound treatment device, and, in step (iv), operate the pump to generate a fourth vacuum pressure at the wound treatment device, wherein the third vacuum pressure is less than or equal to the fourth vacuum pressure.

In an embodiment, the third vacuum pressure is equal or similar to the first vacuum pressure and the fourth vacuum pressure is equal or similar to the second vacuum pressure.

In an embodiment, the third and fourth vacuum pressures provide for effective negative pressure wound therapy.

In an embodiment, after closing the fluid inlet valve and operating the pump to generate the vacuum pressure at the wound, the controller is configured to:
(v) flush the treatment fluid from the wound by:
(v)(a) opening the air inlet valve and operating the pump to maintain a vacuum pressure (e.g. the first vacuum pressure) at the wound treatment device and introduce air into the wound treatment device, and
(v)(b) closing the air inlet valve and operating the pump to maintain a vacuum pressure (e.g. the second vacuum pressure) at the wound treatment device and remove fluid from the wound treatment device.

In an embodiment, in step (v) the controller is configured to repeat steps (v)(a) and (v)(b) a predetermined number of times (for example, three times) to remove treatment fluid from the wound.

In an embodiment, in the fluid treatment state, the controller is configured to repeat steps (iii) to (v) a predetermined number of times.

In an embodiment, the controller is configured to, in step (iv), close the fluid inlet valve, wait for a predetermined time period, and operate the pump to generate the vacuum pressure at the wound treatment device and remove fluid from the wound treatment device.

In an embodiment, the controller is configured to activate the fluid supply state periodically.

In an embodiment, a time period between activating the fluid supply state is much greater than a cycle time of the air inlet valve.

In an embodiment, the system comprises an upstream pressure sensor and/or a downstream pressure sensor in communication with the controller, and, in step (iii), the controller is configured to operate the pump to achieve a vacuum pressure threshold based on a pressure sensed by the upstream and/or downstream pressure sensor.

In an embodiment, the system comprises an upstream pressure sensor and/or a downstream pressure sensor in communication with the controller, and, in step (iv), the controller is configured to operate the pump to achieve a vacuum pressure threshold based on a pressure sensed by the upstream and/or downstream pressure sensor.

This invention may also be said broadly to consist in the parts, elements and features referred to or indicated in the specification of the application, individually or collectively, and any or all combinations of any two or more said parts, elements or features. Where specific integers are mentioned herein which have known equivalents in the art to which this invention relates, such known equivalents are deemed to be incorporated herein as if individually described.

The term ‘comprising’ as used in this specification and claims means ‘consisting at least in part of’. When interpreting statements in this specification and claims that include the term ‘comprising’, other features besides those prefaced by this term can also be present. Related terms such as ‘comprise’ and ‘comprised’ are to be interpreted in a similar manner.

It is intended that reference to a range of numbers disclosed herein (for example, 1 to 10) also incorporates reference to all rational numbers within that range and any range of rational numbers within that range (for example, 1 to 6, 1.5 to 5.5 and 3.1 to 10). Therefore, all sub-ranges of all ranges expressly disclosed herein are hereby expressly disclosed.

As used herein the term ‘(s)’ following a noun means the plural and/or singular form of that noun. As used herein the term ‘and/or’ means ‘and’ or ‘or’, or where the context allows, both.

### BRIEF DESCRIPTION OF THE FIGURES

The present invention will now be described by way of example only and with reference to the accompanying drawings in which:

FIG. 1 is a right-top perspective view showing a first embodiment device having securing tabs;

FIG. 2 is a left-underside side perspective view showing the device of FIG. 1;

FIG. 3 is a top detail view of the device in FIGS. 1 and 2;

FIG. 4 is a side elevation detail view of the embodiment of FIGS. 1 to 3;

FIG. 5 is a detail perspective view corresponding to FIG. 1;

FIG. 6 is a detail perspective view corresponding to FIG. 2;

FIG. 7 is perspective view of an exemplary conduit structure having a truss form;

FIG. 8 is a further perspective view of the conduit structure of FIG. 7;

FIG. 9 is a side detail view of the conduit structure of FIGS. 7 and 8;

FIG. 10 is a side detail view of the truss from the conduit structure of FIGS. 7 to 9;

FIG. 11A is a section view through the truss of FIG. 10, taken through the porous portion of the fluid removal lumen;

FIG. 11B is an end view of the integrally formed portion of the conduit structure of FIGS. 7 to 10 showing the device inlet and outlet;

FIG. 11C is a section view through the conduit structure of FIGS. 7 to 10, taken through the porous portion of the fluid removal lumen;

FIG. 12 is a perspective view of the truss from the conduit structure of FIGS. 7 to 10;

FIG. 13 is a top, cut-away view illustrating of an alternative embodiment device having a truss-type conduit structure;

FIG. 14 is a cut-away perspective corresponding to FIG. 13;

FIG. 15 is a cut-away detail perspective view of the embodiment of FIGS. 13 and 14;

FIG. 16 is a right-top perspective view showing a further embodiment device having a continuous securing flange;

FIG. 17 is a left-underside side perspective view showing the device of FIG. 16;

FIG. 18 is a top detail view of the device in FIGS. 16 and 17;

FIG. 19 is a side elevation detail view of the embodiment of FIGS. 16 to 18;

FIG. 20 is an end view of the embodiment of FIGS. 16 to 19;

FIG. 21 is a section view of through the conduit structure of the device of FIGS. 16 to 20;

FIGS. 22A and 22B illustrate and alternative embodiment conduit structure, where FIG. 22A is a partial perspective section view, and FIG. 22B is a partial top detail view;

FIG. 23 is a cut-away perspective view showing a further embodiment device that is adjustable to shorten the length of the device;

FIG. 24 is a perspective view of the dual lumen conduit of the device of FIG. 23;

FIGS. 25(i) to 25(vi) are partial top views illustrating the process of shortening the device of FIG. 23, where FIG. 25(i) illustrates cutting the device, FIG. 25(ii) shows the cut end of the device, FIG. 25(iii) is a cut-away view showing the cut device, FIG. 25(iv) is a cut-away view showing the conduit structure adjusted to its new position, FIG. 25(v) illustrates flattening the end of the device, and FIG. 25(vi) shows the end folded under to form a seal;

FIG. 26 is a perspective view of one end of an alternative embodiment conduit structure;

FIG. 27 is a top cut-away section view of the conduit structure of FIG. 26;

FIG. 28 is a perspective view of one end of an alternative embodiment conduit structure;

FIG. 29 is a top cut-away section view of the conduit structure of FIG. 28;

FIG. 30 is a perspective view of one end of an alternative embodiment conduit structure;

FIG. 31 is a top cut-away section view of the conduit structure of FIG. 30;

FIG. 32 is a perspective view of one end of an alternative embodiment conduit structure;

FIG. 33 is a top cut-away section view of the conduit structure of FIG. 32;

FIG. 34 is a perspective view of one end of an alternative embodiment conduit structure;

FIG. 35 is a top cut-away section view of the conduit structure of FIG. 34;

FIG. 36 is a perspective view showing a fourth embodiment device with a loop structure;

FIG. 37 is a further perspective view of the embodiment of FIG. 36;

FIG. 38 is a top view of the embodiment of FIGS. 36 and 37;

FIG. 39 is a top cut-away view of the embodiment of FIGS. 36 and 37;

FIG. 40 is a section view of the fluid removal lumen in the embodiment of FIGS. 36 to 39;

FIG. 41 is a cut-away perspective view showing a further embodiment device with a loop structure;

FIG. 42 is a section view taken through the fluid removal lumen of the device of FIG. 41;

FIG. 43 is a plan view of the conduit structure of the device of FIG. 41, illustrating the direction of flow through the device;

FIG. 44 is an end view of the conduit structure of FIG. 43;

FIG. 45 is a plan view of the conduit structure of FIGS. 43 and 44, before assembly within the sheath;

FIG. 46 is a cut-away perspective view showing a further embodiment device with a loop structure;

FIG. 47 is a cut-away plan view corresponding to the embodiment shown in FIG. 46;

FIG. 48 is a section view taken through the fluid removal lumen of the device of FIGS. 46 and 47;

FIG. 49 is a plan view of the conduit structure of the device of FIGS. 46 to 48, illustrating the direction of flow through the device;

FIG. 50 is an end view of the conduit structure of FIG. 49;

FIG. 51 is a plan view of the conduit structure of FIGS. 49 and 50, before assembly within the sheath;

FIG. 52 is a top perspective view showing a fifth embodiment device with a loop structure;

FIG. 53 is an underside perspective view of the device of FIG. 52;

FIG. 54 is a section view of the fluid removal lumen in the embodiment of FIGS. 52 and 53;

FIG. 55 is a top perspective view showing a further embodiment device with a loop structure;

FIG. 56 is a cut-away perspective view of the device of FIG. 55;

FIG. 57 is a top perspective view showing a further embodiment device with a loop structure;

FIG. 58 is a top perspective view showing a further embodiment device with a loop structure with some of the top sheath cut-away;

FIGS. 59A and 59B illustrate directional fluid flow through a sheet of ECM, where FIG. 59A illustrates an exemplary ECM structure prior to processing, and FIG. 59B illustrates the structure following processing and the directional bias of fluid flow through the ECM sheet;

FIG. 60 provides a high-level schematic representation of a negative pressure treatment (NPT) system according to at least one embodiment described herein;

FIG. 61 illustrates the system of FIG. 60 applied to an internal wound;

FIG. 62 is a schematic representation of a vacuum unit of the system of FIG. 60.

FIG. 63 is a schematic representation of the system of FIG. 60;

FIG. 64 is a schematic representation of the system of a further embodiment negative pressure treatment (NPT) system;

FIG. 65 is a schematic representation of a further alternative embodiment of a negative pressure treatment (NPT) system;

FIG. 66 illustrates various flow characteristics with air entrained in a flow of liquid;

FIG. 67 provides a high-level control flow diagram for various embodiments of negative pressure treatment (NPT) system described herein;

FIG. 68 provides a control flow diagram for an airflow state of the control flow diagrams of FIGS. 67 and 72;

FIG. 69 provides a control flow diagram for a pressurise state of the control flow diagrams of FIGS. 67 and 72;

FIG. 70 provides a control flow diagram for a hold pressure state of the control flow diagram of FIG. 67;

FIG. 71 provides a control flow diagram for a timeout state of the control flow diagrams of FIGS. 67 and 72;

FIG. 72 provides a high-level control flow diagram for various embodiments of a NPWT system described herein;

FIG. 73 provides a control flow diagram for a hold pressure state of the control flow diagram of FIG. 72;

FIG. 74 provides a control flow diagram for a fluid flow state of the control flow diagram of FIG. 72;

FIG. 75 provides a control flow diagram for a flushing cycle of the fluid flow state of FIG. 72; and

FIG. 76 provides a chart showing system performance of a treatment system test set-up.

### DETAILED DESCRIPTION

### Definitions

The term “bioresorbable” as used herein means able to be broken down and absorbed or remodelled by the body, and therefore does not need to be removed manually.

The term “treatment site” as used herein refers to a site in a human or animal body where surfaces of muscle tissue, connective tissue or skin tissue have been separated during surgery or as a result of trauma or removal.

The term “propria-submucosa” as used herein refers to the tissue structure formed by the blending of the lamina propria and submucosa in the forestomach of a ruminant.

The term “lamina propria” as used herein refers to the luminal portion of the propria-submucosa, which includes a dense layer of extracellular matrix.

The term “extracellular matrix” (ECM) as used herein refers to animal or human tissue that has been decellularised and provides a matrix for structural integrity and a framework for carrying other materials.

The term “decellularised” as used herein refers to the removal of cells and their related debris from a portion of a tissue or organ, for example, from ECM.

The term “helical” as used herein refers to a generally spiralling form, it may relate to a form with a circular cross-section, but also refers to forms with non-circular cross sections.

The term “polymeric material” as used herein refers to large molecules or macromolecules comprising many repeated subunits, and may be natural materials including, but not limited to, polypeptides and proteins (e.g. collagen), polysaccharides (e.g. alginate) and other biopolymers such as glycoproteins, or may be synthetic bioresorbable materials including, but not limited to polyglycolic acid, polylactic acid, P4HB (Poly-4-hydroxybutyrate), polylactic and polyglycolic acid copolymers, polycaprolactone, polydioxanone and poly(trimethylene carbonate) or they may be non-absorbable materials such polypropylene, polyester, polytetrafluoroethylene, polyamide and polyethylene.

### Device

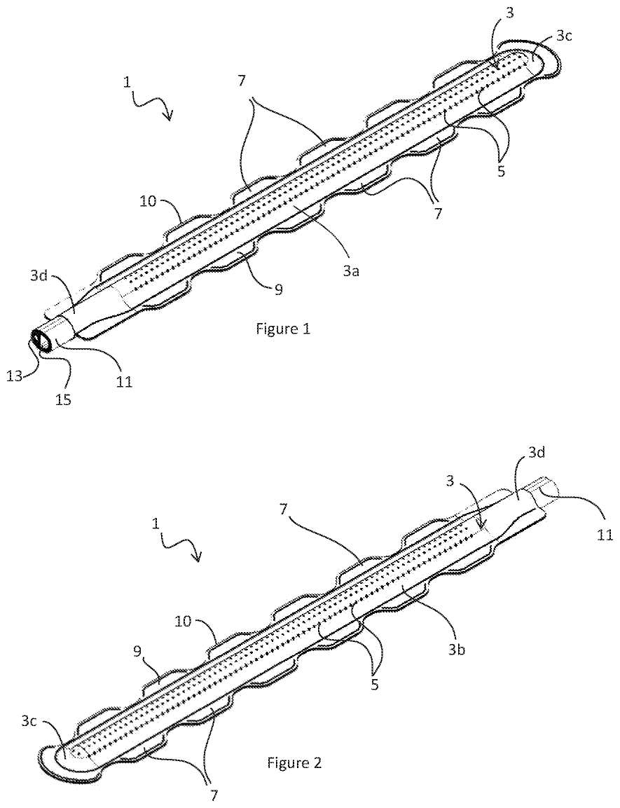

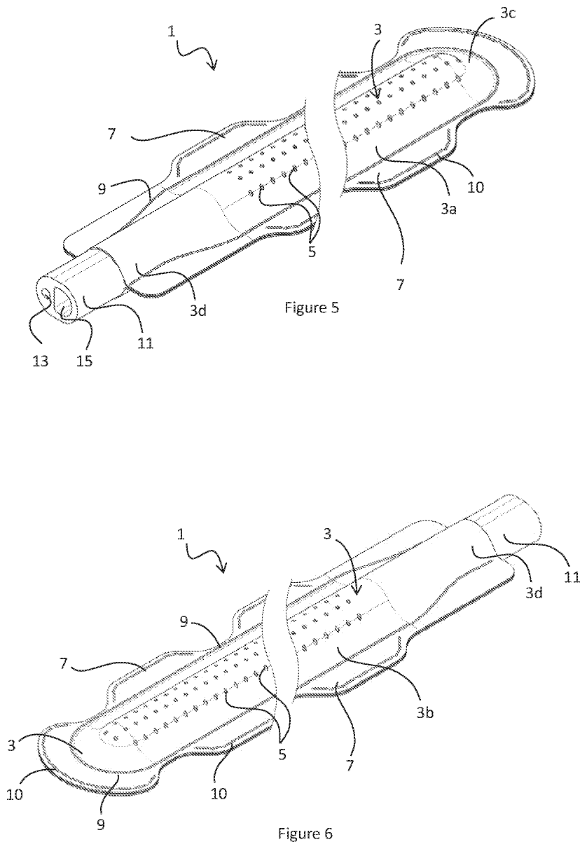

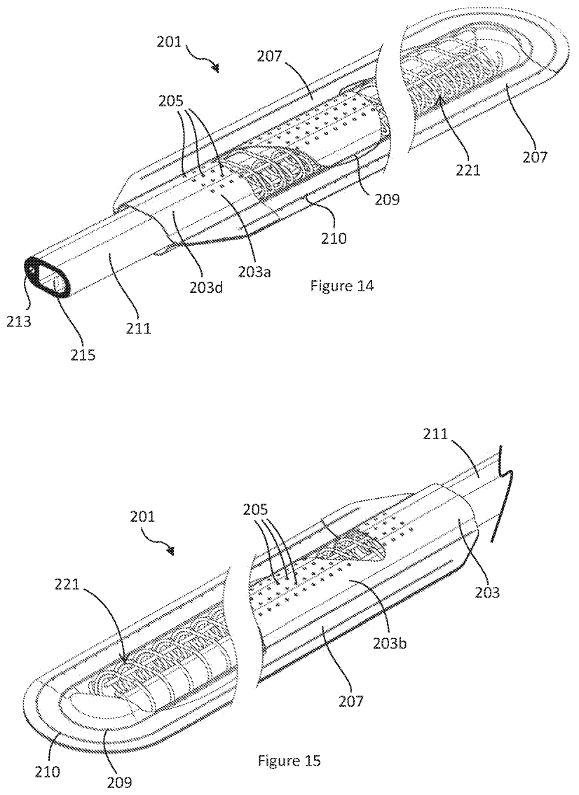

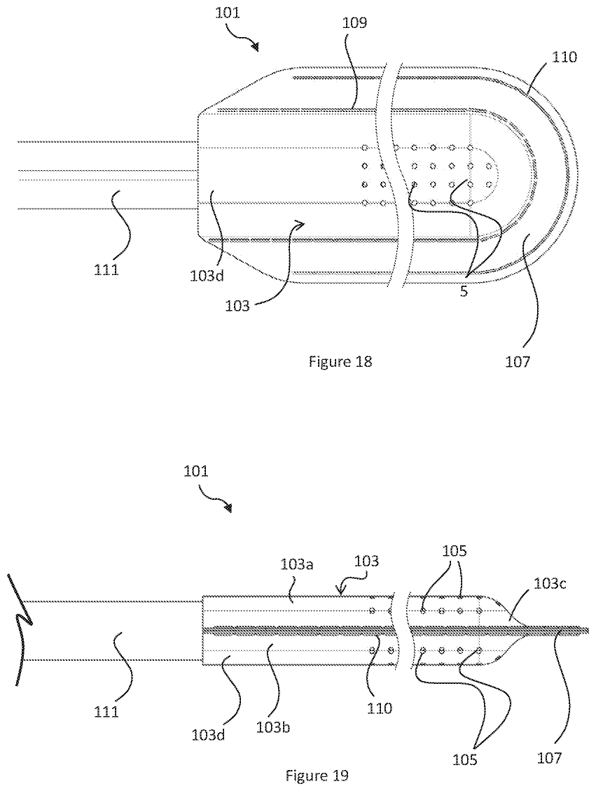

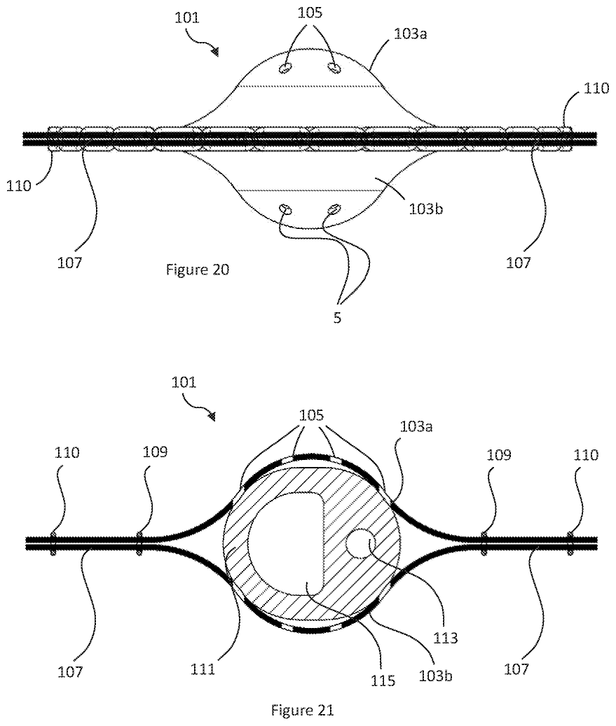

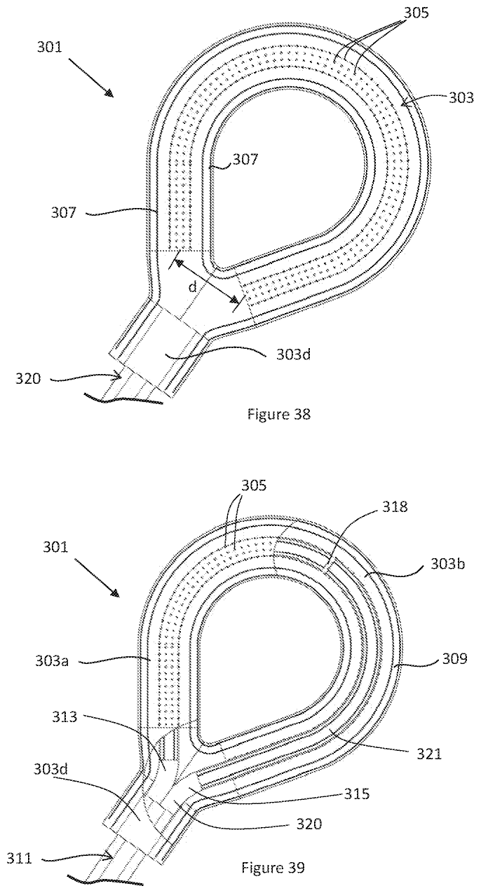

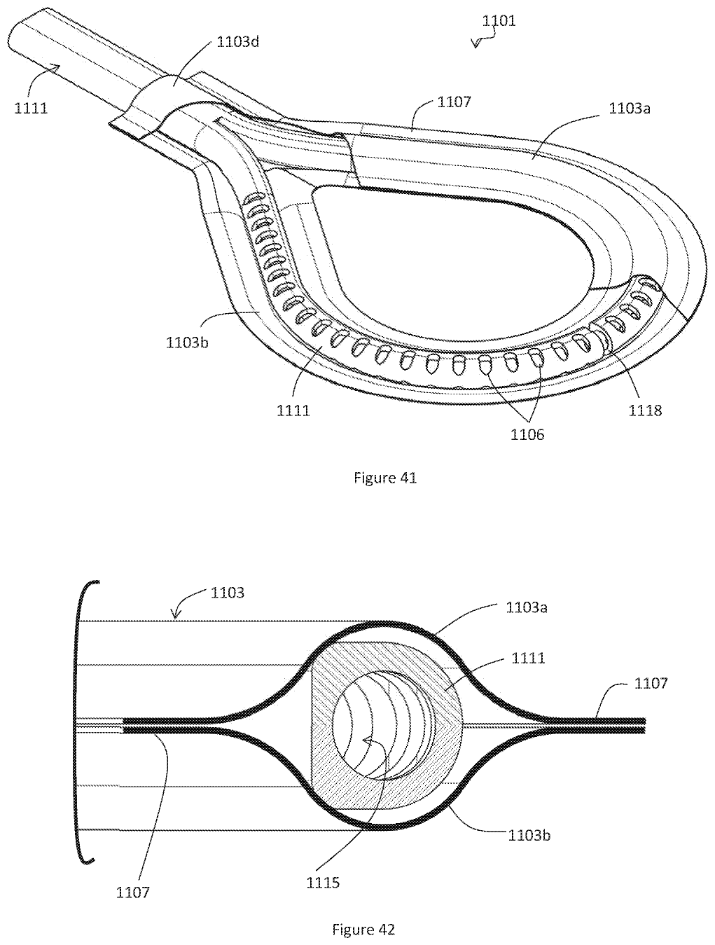

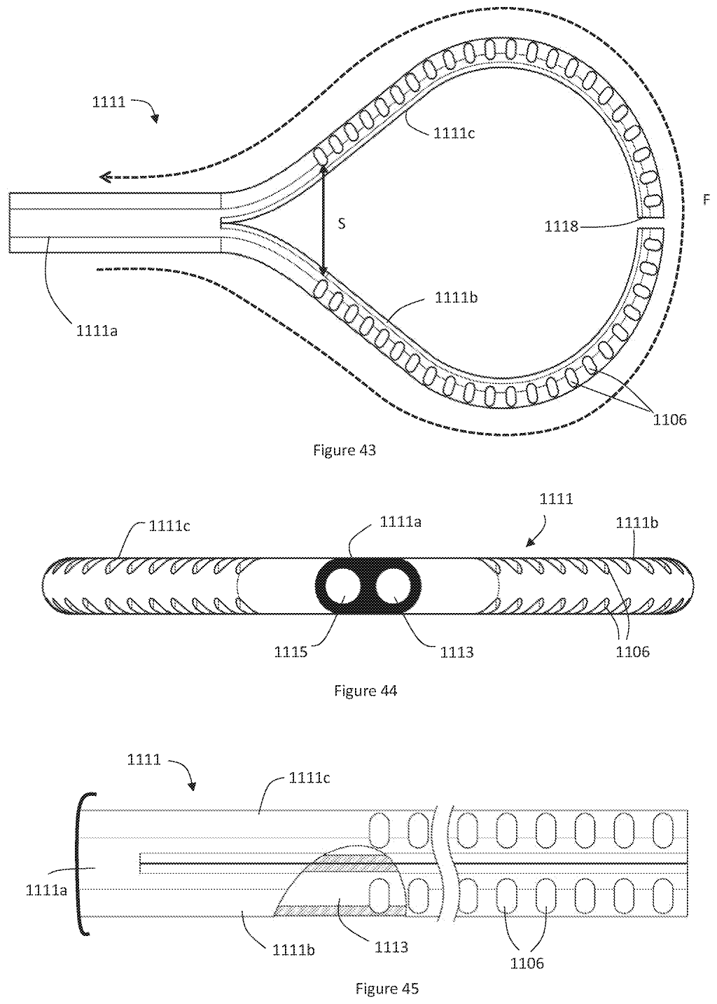

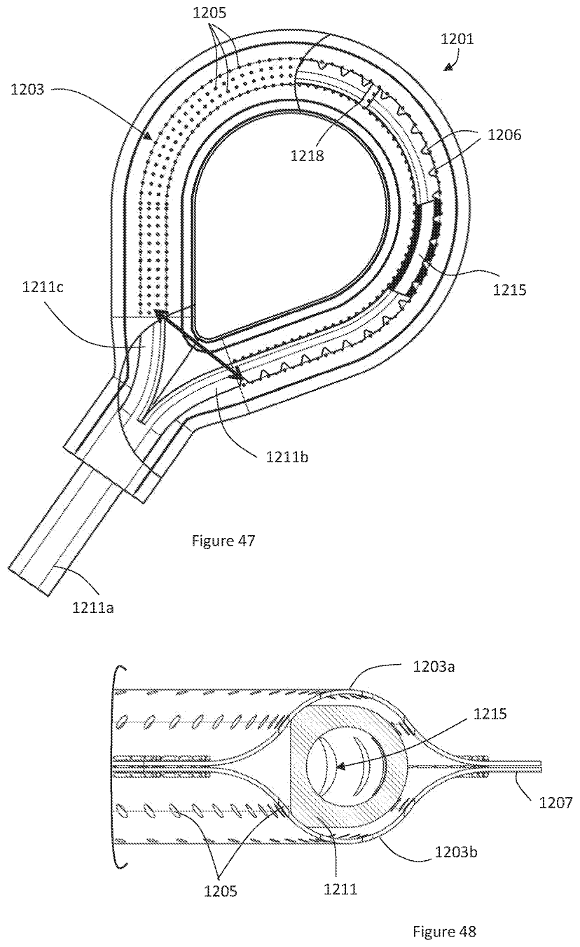

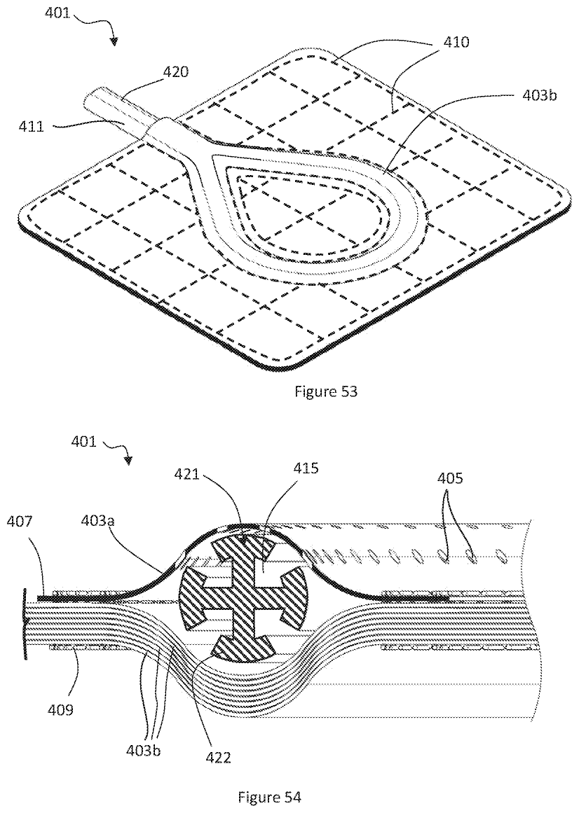

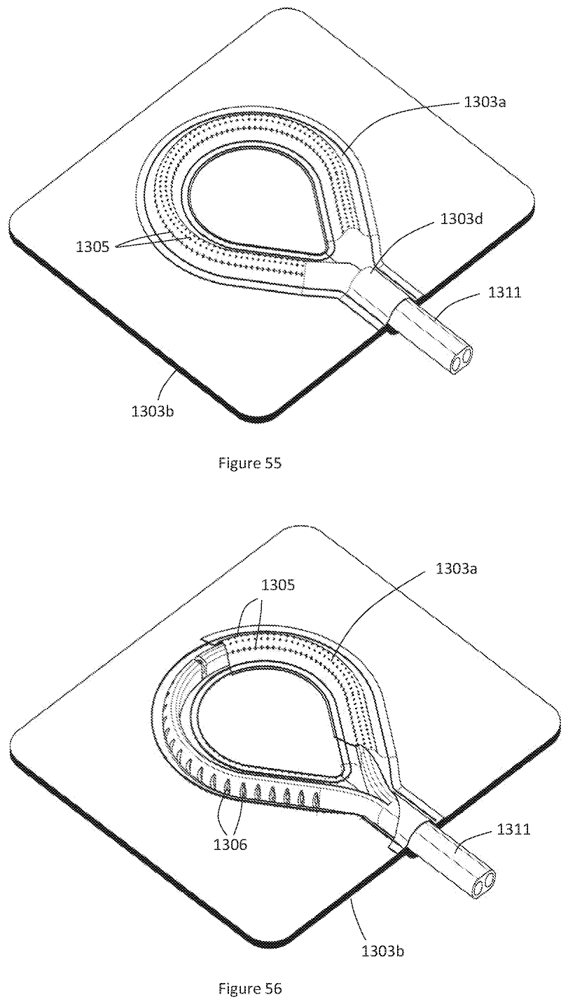

Various embodiments of the device and system of the present invention will now be described with reference to FIGS. 1 to 59B. In these figures, unless otherwise described, like reference numbers are used to indicate like features. Where various embodiments are illustrated, like reference numbers may be used for like or similar features in subsequent embodiments but with the addition of a multiple of 100, for example 2, 102, 202, 302 etc.

Directional terminology used in the following description is for ease of description and reference only, it is not intended to be limiting. For example, the terms ‘front’, ‘rear’, ‘upper’, ‘lower’, and other related terms are generally used with reference to the way the device is illustrated in the drawings.

FIG. 1 illustrates one embodiment device 1 for implantation at a treatment site in the body of a patient for delivering fluid to the treatment site and also for draining fluid from the treatment site. The drained fluid may include the treatment fluid and/or wound exudate. The device comprises a bioresorbable porous sheath 3 that surrounds a removable conduit structure 11. The conduit structure 11 acts to hold apart two tissue surfaces of the wound treatment site to create a channel for delivering and removing fluid.

The device 1 is a flexible device such that the device can generally conform to the contours of a wound site. The device may be elongate, but may have other forms.

The conduit structure 11 is a flexible structure comprising a material that is non-resorbable by a body, such that the conduit structure is configured to be removed at the end of the treatment. The conduit structure defines a fluid supply lumen 13 and a fluid removal lumen 15. The fluid supply lumen and fluid removal lumen may be positioned side-by-side or may be coaxial.

The fluid supply lumen 13 is a generally closed wall lumen configured to supply a fluid to an inlet end of the fluid removal lumen. In contrast, the fluid removal lumen 15 has a generally porous wall along a length of the lumen, to allow fluid communication between the fluid removal conduit and the treatment area. The fluid removal lumen may have a circular or non-circular cross section. The fluid removal lumen has a cross-sectional area of at least 16 mm2, for example an area of 18 mm2.

The bioresorbable sheath 3 surrounding the conduit structure comprises a plurality of apertures 5 positioned to enable fluid communication across the sheath 3, between the treatment site and the conduit structure. The apertures 5 each have an area of about 1 mm² or less, preferably about 0.8 mm² or less, for example between about 0.2 mm² and about 0.5 mm². If the apertures are too small, the device 1 may be ineffective for prevention of seroma formation. If the apertures 5 are too large, wound debris such as fatty tissue may be drawn into the device and cause blockages.

The fit of the sheath 3 over the conduit structure 11 should be tight to ensure the sheath isn't sucked into the fluid removal lumen on the application of negative pressure, and to minimise the likelihood of wound debris entering the conduit structure other than through the sheath apertures 5. In preferred embodiments, the sheath 3 comprises top and bottom sheets 3a, 3b that wrap over and sandwich the conduit structure 11 between the sheets. The top and bottom sheets 3a 3b are joined together along a side seam 9, along the side of the conduit structure 11, the side seam 9 may comprise one or more rows of stitching, for example.

The sheath 3 comprises one or more flange, or tabs 7 for securing the device 1 to the wound treatment site, for example by suturing the flange or tab 7 to tissue at the wound treatment site. This ability to secure the device enables accurate placement of the device 1 at the wound site, and reduces the likelihood of the device moving away from the installed position, particularly for treatment sites that undergo high levels of movement. Securing the sheath 3 of the device 1 to tissue at the treatment site also allows the removable conduit structure 11 to be removed while minimising movement of the sheath 3, and thereby reduces disruption to surrounding tissue which may have bonded with the sheath 3.

The flange or tabs 7 extend out beyond the side seam 9, and preferably comprise both the top and bottom sheath layers 3a, 3b to provide a stronger connection with the securing sutures and to stiffen the flange or tabs to improve the ease of stitching. The flange or tabs 7 may be stitched together at or near and edge of the flange of tab 7 along a peripheral stitch line 10 to prevent the sheets 3a, 3b separating.

In some embodiments of the invention, the sheath 3 is formed from extracellular matrix (ECM). The ECM sheets are typically collagen-based biodegradable sheets comprising highly conserved collagens, glycoproteins, proteoglycans and glycosaminoglycans in their natural configuration and natural concentration. ECM can be obtained from various sources, for example, dermis pericardial or intestinal tissue harvested from animals raised for meat production, including pigs, cattle and sheep or other warm-blooded vertebrates.

The ECM tissue suitable for use in the invention comprises naturally associated ECM proteins, glycoproteins and other factors that are found naturally within the ECM depending upon the source of the ECM. One source of ECM tissue is the forestomach tissue of a warm-blooded vertebrate. The ECM suitable for use in the invention may be in the form of sheets of mesh or sponge.

Forestomach tissue is a preferred source of ECM tissue for use in this invention. Suitable forestomach ECM typically comprises the propria-submucosa of the forestomach of a ruminant. In particular embodiments of the invention, the propria-submucosa is from the rumen, the reticulum or the omasum of the forestomach. These tissue scaffolds typically have a contoured luminal surface. In one embodiment, the ECM tissue contains decellularised tissue, including portions of the epithelium, basement membrane or tunica muscularis, and combinations thereof. The tissue may also comprise one or more fibrillar proteins, including but not limited to collagen I, collagen III or elastin, and combinations thereof. These sheets are known to vary in thickness and in definition depending upon the source of vertebrate species.

The method of preparing ECM tissues for use in accordance with this invention is described in U.S. Pat. No. 8,415,159.

In some embodiments of the invention, sheets of polymeric material may be used. The polymeric material may be in the form of sheet or mesh. Synthetic materials such as polyglycolic acid, polylactic acid and poliglecaprone-25 will provide additional strength in the short-term, but will resorb in the long term. Alternatively, the polymeric material may be a natural material, or derived from a natural material, such as a proteins (e.g. collagen), a polysaccharides (e.g. alginate), and a glycoprotein (e.g. fibronectins).

Any desirable bioactive molecules can be incorporated into the ECM or polymeric material. Suitable molecules include for example, small molecules, peptides or proteins, or mixtures thereof. The bioactive materials may be endogenous to ECM or maybe materials that are incorporated into the ECM and/or polymeric material during or after the grafts manufacturing process. In some embodiments, two or more distinct bioactive molecules can be non-covalently incorporated into ECM or polymer. Bioactive molecules can be non-covalently incorporated into material either as suspensions, encapsulated particles, micro particles, and/or colloids, or as a mixture thereof. Bioactive molecules can be distributed between the layers of ECM/polymeric material. Bioactive materials can include, but are not limited to, proteins, growth factors, antimicrobials, and anti-inflammatories including doxycycline, tetracyclines, silver, FGF-2, TGF-B, TGF-B2, BMR7, BMP-12, PDGF, IGF, collagen, elastin, fibronectin, and hyaluronan.

FIGS. 1 to 6 show a first exemplary embodiment device 1 having a sheath 3 with an upper sheet 3a, and a lower sheet 3b. As described previously, the upper and lower sheets 3a, 3b are joined at a sewn seam 9 along the side of the conduit structure 11, with tabs 7 protruding from the seam. Apertures 5 are provided in both the top 3a and bottom 3b sheath sheets. Referring to FIG. 4, the apertures 5 are distributed such that some of the apertures 5 are positioned on a side of the device. These side apertures may be helpful in some applications to allow negative pressures to be continued to be applied to the treatment site if the top apertures are in contact with a tissue surface. In the embodiment shown, the two outer rows of apertures 5 on the upper sheet 3a open to an angle upwards and outwards, and the two outer rows of apertures 5 on the lower sheet 3b open to an angle downwards and outwards.

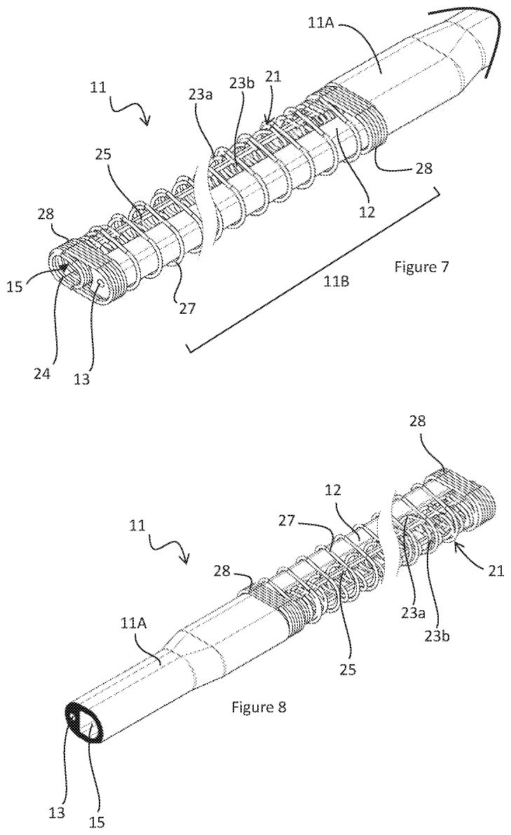

The device 1 has an elongate shape with both the inlet and outlet ports for the device provided at the same end, and with an opposite closed end 3c of the device. The sheath 3 comprises a sealing end section 3d at the end of the sheath proximal the inlet and outlet, where the conduit 11 protrudes from the sheath 3. This end section 3d is free from apertures and extends over a portion of the conduit structure 11A that comprises fluid impervious walls (Further illustrated in FIGS. 7 to 10). This end section 3d forms a tight fit with the underlying conduit structure 11 and acts to provide a type of seal with the conduit structure 11 that prevents or reduces the ingress of wound debris, tissue debris and fat between the sheath 3 and conduit structure 11, which has the potential to cause blockages.

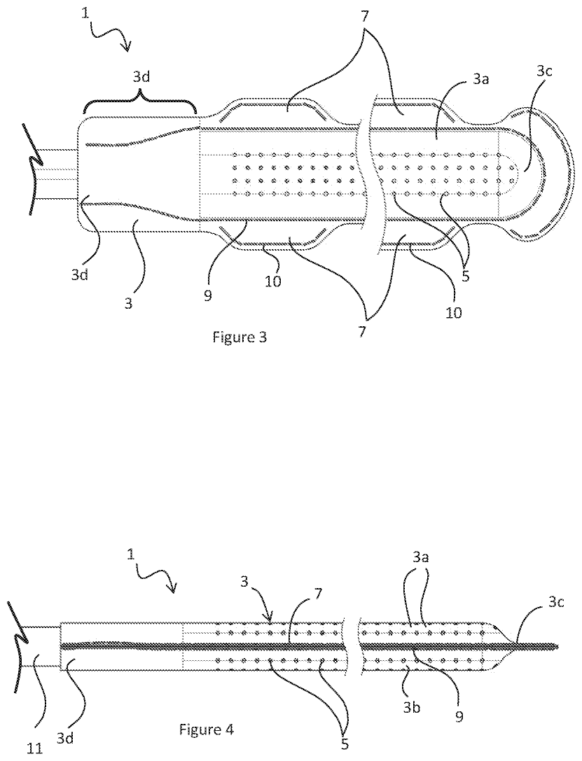

The conduit structure 11 and sheath 3 may neck at or along the sealing end section 3d of the sleeve 3 to create a smaller cross section at the opening of the sheath 3, as best illustrated in FIG. 3. This necked section further enhances the seal between the sheath 3 and the outer surface of the conduit structure 11 at the end region 3d and improves the retention of the conduit structure 11 within the sheath 3. This configuration is particularly intended for use in embodiments in which the conduit structure comprises a truss, as described in more detail below.

In some embodiments the seal between the sealing end section 3d and the conduit structure 11 could be further improved by lengthening this end section 3d. Additionally, or alternatively, the sheath 3 could be tied to the conduit structure 11 at this section 3d, using a noose-type tie, tightly wrapping around the sheath. In some embodiments, an additional sheet of bioresorbable material may be wrapped around the conduit 11 at this end section 3d to improve the seal.

In the embodiment shown in FIG. 1, the device 1 comprises a removable conduit structure 11 having the fluid supply lumen 13 and removal lumen 15 arranged side-by-side. In this embodiment, a length of the conduit structure 11 (including the length external of the sheath) consists of an extruded dual lumen conduit. The fluid supply lumen 13 is a generally closed wall tube with fluid impervious walls, that is positioned in the sheath 3 to deliver fluid to the distal, closed end 3c of the device 1 and thereby to the inlet end of the fluid removal lumen 15. The outlet end of the inlet lumen 13 is adjacent to and in fluid communication with the inlet end of the fluid removal lumen 15. Since no part of the fluid supply lumen 13 is in fluid communication in a downstream direction from the wound site, supplied fluid is delivered to the fluid removal lumen 15 consistently without blockages occurring in the supply lumen 13.

FIGS. 7 to 12 illustrate one exemplary embodiment conduit structure 11 for use in the embodiment of FIGS. 1 to 6. In this embodiment, the conduit structure comprises two portions, a first integrally formed portion 11A positioned proximal the first end of the sheath 3, and protruding from the sheath, and a second portion 11B wholly contained in the sheath 3.

The integrally formed portion 11A of the conduit structure 11 comprises a dual lumen conduit defining a first portion of the inlet lumen 13 and a second portion of the outlet lumen 15 and forming the inlet and outlet to the device 1. The lumens 13, 15 of the integrally formed portion 11A comprise impervious walls with no through apertures. Typically, the inlet lumen 13 for the fluid supply is significantly smaller than the larger fluid removal lumen 15. The integrally formed portion 11A may be a moulded piece and preferably formed from a material such as silicone.

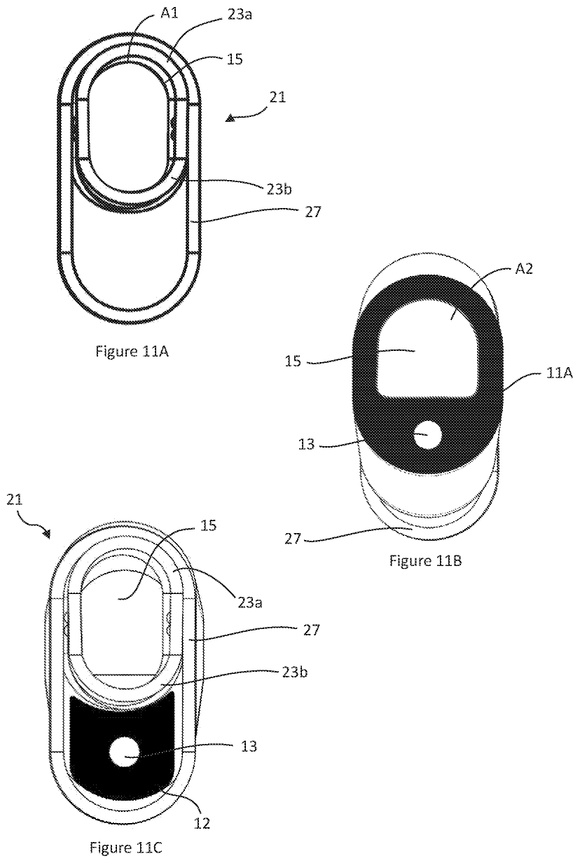

The second portion 11B of the conduit structure 11 comprises a separate fluid supply conduit 12 defining a second portion of the fluid supply lumen 13, and a flexible truss structure 21 defining a first portion of the fluid removal lumen 15. The fluid supply conduit 12 is arranged to be in fluid communication with the fluid supply lumen of the integrally formed portion 11A, preferably with the first and second portions of the fluid supply lumen arranged coaxially. The fluid supply conduit 12 may be an extruded component having fluid impervious walls, for example formed from a material such as silicone. Referring to FIG. 11C, the exterior of the fluid supply conduit 12 may be shaped to complement the truss structure 21.

The flexible truss structure 21 forms the walls of the porous section of the fluid removal conduit 15. The truss 21 is tubular in nature, with a non-circular or circular cross section (in this embodiment the truss defines a lumen 15 with a substantially oval cross section). The truss 21 is configured to, in use, provide support to the surrounding tissue surfaces in all generally radial directions. The truss 21 is flexible in its longitudinal and traverse directions to allow the channel(s) to flex to substantially conform to the contours of the treatment site while having sufficient strength to hold two tissue surfaces apart, at least at the time of implantation, without the truss buckling or the channel collapsing or kinking under movement or application of clinically appropriate levels of negative pressure. The truss 21 is preferably relatively incompressible in the longitudinal direction of the truss 21.

The truss 21 comprises two flexible elongate wall members 23a, 23b, which are wound in a manner to form a framework for, and thereby define, the fluid removal lumen 15 into which fluid from the treatment site can drain or from which fluid can be delivered to the treatment site. The wall members are wound such that they intersect each other periodically at a plurality of cross-over nodes. The wall members 23a, 23b are most commonly helically wound, with the two wall members having opposite (left-hand and right-hand) winds. Alternatively, the truss 21 may comprise helical members wound in the same direction but with different pitches, or a plurality of wall members of an alternative non-helical repetitive shape, such that the wall members periodically intersect each other at cross-over nodes.

The truss 21 further comprises at least two flexible elongate bracing members 25, each bracing member is bonded or linked to the two elongate wall members 23a, 23b at a plurality of the wall member cross-over nodes forming periodic interlocked points along the truss, for example by way of heat bonding. In preferred embodiments, each bracing member 25 extends generally longitudinally along a wall of the outlet lumen 15. These bracing members 25 act to hold the periodic cross-over nodes of the wall members 23a, 23b in spaced apart relation, to reduce or prevent collapse of the lumen walls due to relative movement of these points, thereby preventing or minimising the likelihood of crushing and kinking.

Finally, one or more securing truss members 27 is provided to secure the separate fluid supply conduit 12 alongside the truss 21 defining the removal lumen 15. The securing truss member 27 in the embodiment shown comprises a helical member 27 that is wound about the outside of the fluid removal truss 21 defining the outlet lumen 15 and the separate fluid supply conduit 12. This securing truss member 27 also advantageously spaces the sheath 3 from the impervious wall of the fluid supply conduit 12, thereby creating a fluid path over the wall of the fluid supply conduit 12 to the fluid removal lumen 15. This enables fluid supply to and removal from across the full width of the device 1.

The wall truss members 23a, 23b, and the securing truss member 27, may be tightly wound at respective end portions 24, 28, 29. These tightly wound portions 24, 28, 29 anchor the helical members and facilitate connection between the various device components such as coupling with the integrally formed portion 11A.

To manufacture the truss 21, in a first step, a filament is clamped at one end by a clamp and wound around a first rod-like mandrel in a helical manner at a first pitch length to form part of the first end portion 29. The filament is then further wound around the first mandrel in a helical manner at a second pitch length to form a first wall member 23a with the filament then clamped in place at the opposing end. Two elongate bracing members 25 are then also clamped at their ends by the clamp and laid over the first wall member 23a, along opposing sides of the mandrel, typically top and bottom. A second filament is then clamped by the clamp and wound around the first rod-like mandrel at a first pitch length in the opposite direction to the first wall member 23a to form the second end portion 24. The second filament is then further wound around the mandrel at a second pitch length to form a second wall member 23b. Upon reaching the first part of the first end portion 29, the second filament is then wound at a third pitch length to complete the tight wind of the first end portion 29. The first helical member, bracing members, second helical member and the first rod-like mandrel to form an inner truss.

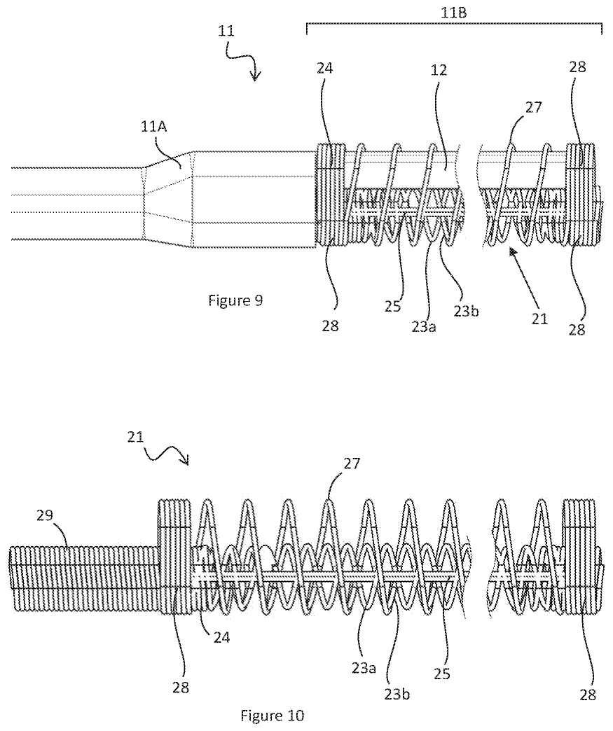

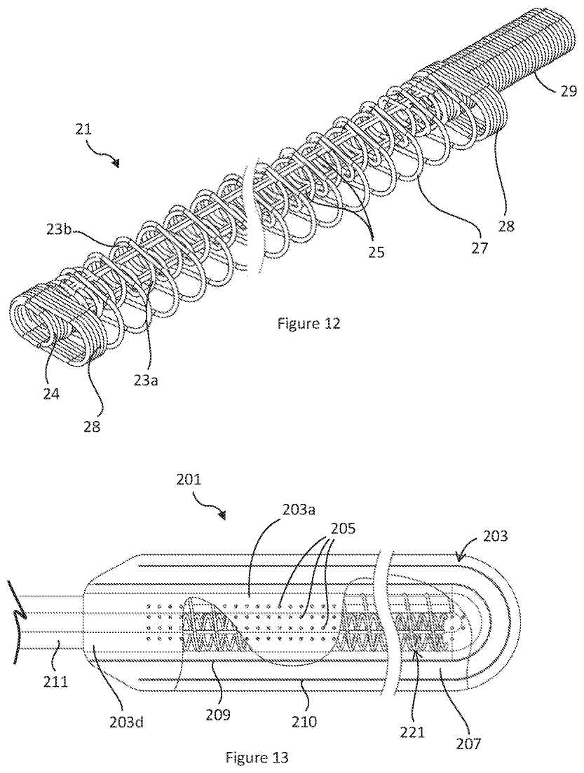

In a next step, a second mandrel is positioned alongside the inner truss and first rod-like mandrel. A filament for forming the securing member 27 is clamped and wound in a helical like manner in a first pitch length to produce a tightly wound first end portion 28. The filament is then further wound in a helical like manner at an increased pitch length to form the securing truss member 27 along a majority of the length of the truss before being tightly wound to form the second end portion 28 at the opposite end to the first end portion 28. The wound filaments are then heated to fuse the bracing members 25 to the first and second wall members 23a, 23b at the points where they overlap. The truss is allowed to cool, thereby setting the shape of the truss members. After cooling the clamp and mandrel are removed leaving the hollow truss as shown in FIGS. 10 and 12. The end 11A of the extruded adjoining removal lumen is pushed over the end portion 29 of the truss structure 21 and the extruded fluid supply lumen 12 is inserted, to form the structure shown in FIG. 9. It will be apparent that the order of the method steps may vary, and that not all steps are necessary.

The truss members 23a, 23b, 25, 27 preferably comprise a non-absorbable polymer filament such as monofilament polypropylene, however, any suitable absorbable or non-absorbable polymer can be used. Preferably the filaments are selected such that the filament can be heated to a melting point without excessive melting occurring that would measurably modify the mechanical properties of the filament.

FIGS. 13 to 15 illustrate a second embodiment device 201, comprising a truss-type conduit structure 221 similar the truss structure 21 described above, but within an alternative form sheath 203. In this device 201, the sheath 203 comprises a single flange 207 that protrudes from a midline of the device, rather than a plurality of tabs 7. The singular flange 207 advantageously allows for better securement when the device 201 is used in undulating sites or sides containing discrete areas that the device cannot be attached to such as bone.

In this embodiment, the open end 203d of the sheath 203 has a constant width and does not narrow. This provides for easier assembly compared to an embodiment that narrows at the inlet.

In devices such as those described above having a linear conduit structure, rather than being sandwiched between two sheets of bioresorbable material, the sheath may comprise a single sheet of bioresorbable material wrapped around the conduit structure and joined along one side the conduit structure. A securement flange may be created along the opposite sides of the conduit by folding the resorbable sheet and sewing along and in from the fold.

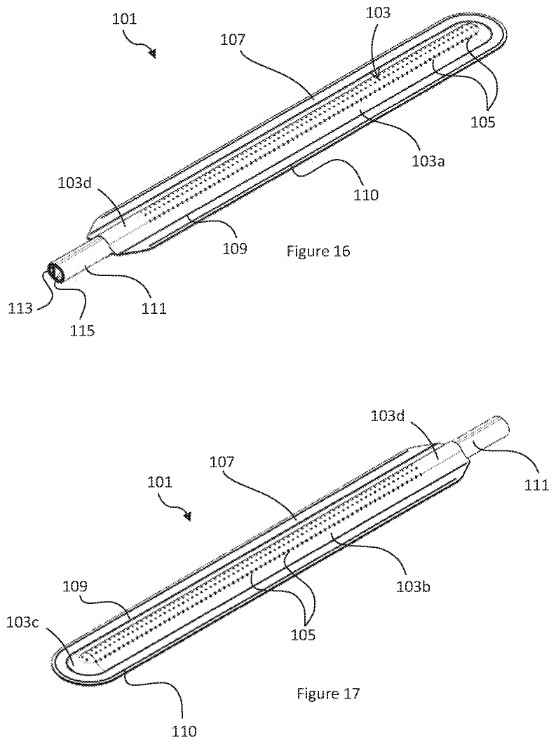

In alternative embodiments, the conduit structure may have alternative forms. FIGS. 16 to 22B illustrate a second embodiment device 101 in which full length of the conduit structure 111 comprises a flexible dual lumen extrusion, in contrast to the truss-comprising structure described previously.

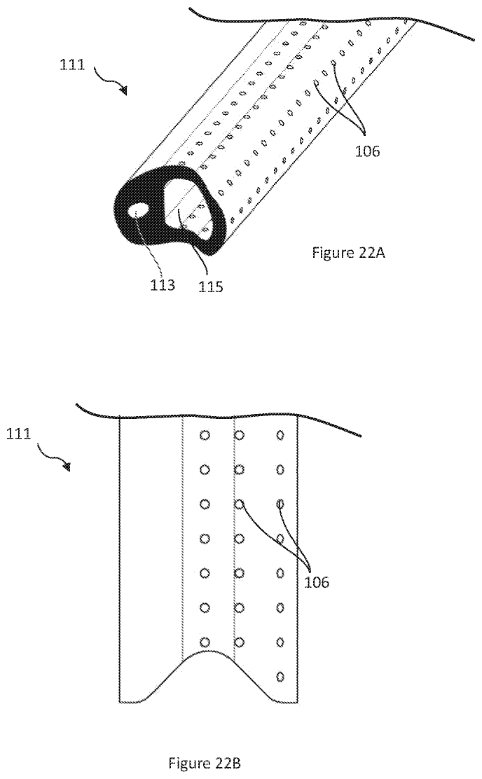

The dual lumen extrusion 111 is typically formed from a material such as silicone, with the fluid supply lumen 115 and fluid removal lumen 113 formed side-by-side. Referring in particular to the section view of FIG. 21, in this embodiment, the fluid supply lumen 113 has a circular cross-section and fluid impermeable walls. The fluid removal lumen 115 is significantly larger than the fluid supply lumen 113 and is D-shaped in cross section. The D-shape provides for an increased removal lumen volume compared with a circular cross-section, given the outer diameter of the conduit structure 111. Apertures 106 are provided in the wall of the fluid removal conduit to allow the egress and ingress of fluid into and from that channel. The apertures 106 each have an area of about 0.5 mm².

The inlet end 103d of the sheath 103 is a constant width and does not narrow towards the device inlet, this facilitates easy removal of the conduit structure 111 when required.

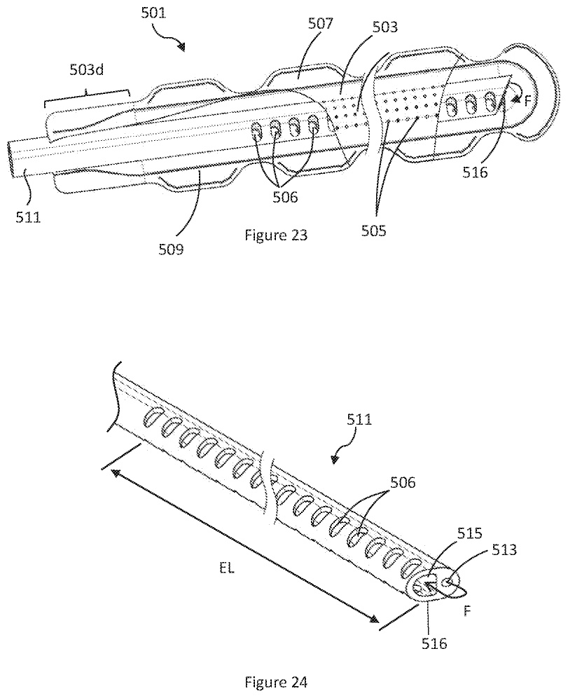

FIGS. 23 to 25(vi) illustrate an alternative form device 501 that is adjustable in length to customise the device to fit various wounds. The device 501 is provided in a first length illustrated in FIG. 23, for shortening as required to fit a smaller treatment site.

This embodiment device 501 comprises a sheath 503 that is substantially the same as the sheath 3 of the first embodiment device 1. The conduit structure 511 comprises a flexible dual lumen extrusion shown in FIG. 24 having a fluid supply lumen 513 with fluid impervious walls, and a larger fluid removal lumen 515. The walls of the fluid removal lumen 515 comprise two oppositely positioned rows of apertures 506 along a length TD of the conduit structure 511. The apertures 506 in this embodiment conduit 511 are larger than those of the previous embodiment 111 and so are more likely to overlap with the smaller apertures 505 of the sheath 503. As such the larger apertures 506 may provide improved exchange of fluid from the treatment site into the conduit.

The apertures 506 are provided along a length EL of the device that is typically shorter than the length of the sheath. This length EL of the device must be contained within a sealed environment (i.e. within the sealed treatment site) to ensure the vacuum is maintained. In addition, for this embodiment, in which the apertures on this conduit are larger than a minimum threshold dimension for preventing blockages (for example, 0.5 mm²), they must also be contained within the sheath 503 of the device 501 to prevent blockages.

The distal end of the conduit structure 511 comprises an angled surface 516, angled to position the outlet of the fluid supply lumen 513 further along the device than the inlet to the fluid removal lumen 515. This angled surface creates a cavity within the sheath 503 between the surface 516 and the end of the sheath 503c, to accommodate fluid flow F from the outlet of the fluid supply lumen 513 to the fluid removal lumen 515.

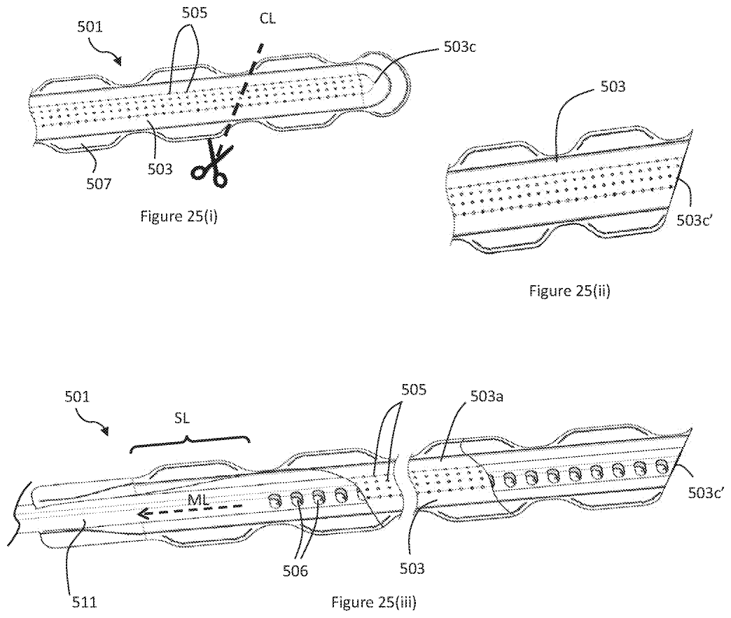

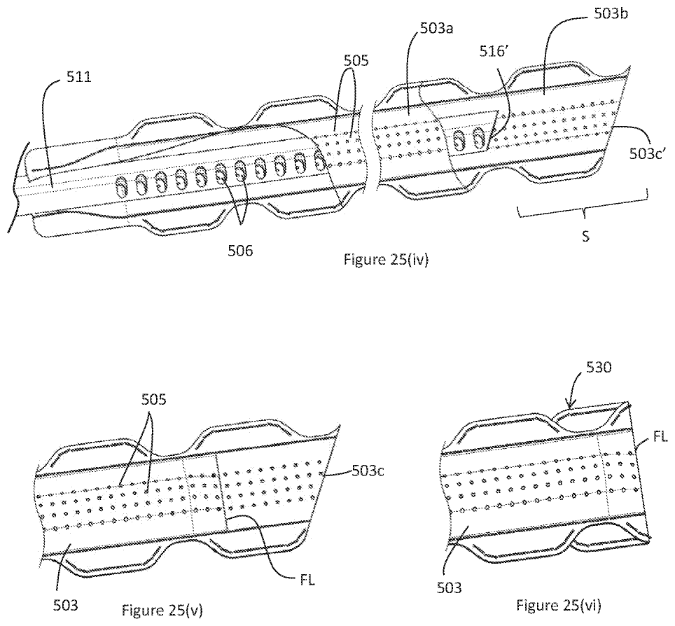

FIGS. 25(i) to 25(vi) illustrate the process of shortening the length of the device 501. In a first step illustrated in FIGS. 25(i) to 25(iii), the device 501 is cut along a cut line CL. The cutline CL is at an angle to the longitudinal direction of the device and should be substantially parallel with the angled end 516 surface of the conduit structure 511. Cutting the device 501 at an angle in this manner ensures that the shortened device will retain the cavity within the sheath 503 between the surface 516 and the end of the sheath to accommodate fluid flow F. The position of the cutline CL should be selected to be slightly longer than the desired length of the device to accommodate the sealing of the sheath end as will be described below.

In a second step, the conduit structure 511 is pulled in the direction of the inlet, as illustrated by the like ML of FIG. 25(iii), relative to the sheath. This creates a spacing S between the cut, angled end 516′ of the conduit structure 511 and the cut end 503c′ of the sheath 503 as illustrated in FIG. 25(iv). To accommodate this anticipated movement of the conduit structure 511 within the sheath to resize the device, the conduit structure may have a sealed length SL free from apertures 506. This length SL ensures the large apertures 506 are not pulled beyond the sheath or to a position that may compromise the seal of the device or result in blockages when the device is shortened.

To mitigate this, and to provide a device with a longer effective length EL, small apertures having a dimension smaller than a blocking threshold may optionally be provided along the length SL. A longer effective length EL is advantageous because it increases the effective treatment area of the device, which is determined by distance to the nearest aperture 506. As one example, in the embodiment of FIG. 23, the device may only provide treatment to within about 25 mm to the nearest aperture.

Referring to FIGS. 25(v) and 25(vi), in a final step, the excess material 530 at the end of the sheath 503 is flattened and folded over along a perpendicular fold line FL to close the end of the sheath. The folded portion 530 is secured to tissue at the treatment site to prevent any unwanted tissue ingress into through the cut end of the device.

In one embodiment, the device illustrated in FIGS. 25(i) to 25(vi) may be provided as separate components for assembly by a clinician before use. In such an embodiment, the sheath 503 may be in a generally tubular form having two open ends, with the proximal section 503d substantially as described herein in relation to the various embodiments, with the opposing distal end 503c open, in a form similar to that shown in FIG. 25 (ii). The open end at 503c may have any suitable shape, such as a square edge or an angled edge. The end edges of the top and bottom sheaths may align (as shown in FIG. 25(ii), or one sheath of the device may extend beyond the other sheath to facilitate assembly with the conduit structure. This alternative embodiment device may be assembled following the steps shown in FIGS. 25(iii) to 25(v) within the clinical setting prior to implantation, with the distal end of device 503c folded over and secured place (as shown in FIG. 25(vi)) during implantation.

FIGS. 22A and 24 in particular exemplify two alternative non-resorbable dual-conduit type structures for use in the devices of FIG. 23. However, alternative designs are envisaged. In the embodiment of FIGS. 23 to 25(vi) the fluid removal lumen 515 has a D-shaped cross-sectional with an area of about 18 mm2, and the fluid supply lumen has a circular cross section with a diameter of about 1.4 mm. However, other sizes and shapes for these lumens are envisaged. For example, the inlet lumen may have a diameter from about 1 mm to about 2 mm.

In embodiments where the conduit structure 111 of FIGS. 22A and 22B is used, the small 0.5 mm apertures 106 enable the apertures to cover a longer length of the device. When adjusting the length of the device, the conduit structure 111 may be pulled so that some of the apertures are in the sealing region 503d of the sheath, or even outside of the sheath, but the small size of the apertures prevents the blocking of the fluid removal lumen.

In a further embodiment, small sized apertures 106 may only be provided in a localised zone near the sealing end 503d of the sheath (but internally in the sheath). The remaining length of the fluid removal conduit may include larger apertures to provide a higher degree of fluid exchange between the conduit and the treatment area. This mean that if a user inadvertently removes too much of the tube from the sleeve during the steps to shorten the device such that some of the apertures are outside of the sheath, the small size of the apertures prevents this resulting in blocking of the fluid removal lumen.

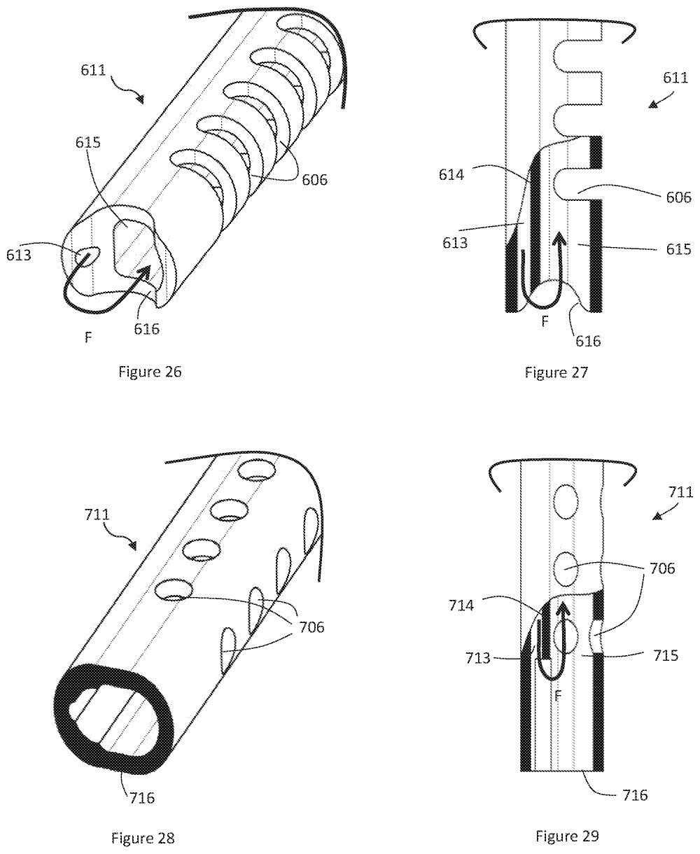

As examples, FIGS. 26 to 35 illustrate some further embodiment conduit structures. FIGS. 26 and 27 illustrate one embodiment conduit structure 611 having a fluid supply lumen 613 and a fluid removal lumen 615, separated by an internal call 614. In this embodiment, the fluid removal lumen 615 comprises a row of slot-like apertures 606. The apertures 606 follow the curvature of the lumen wall and their larger size improves the passage of fluid from the treatment site into the fluid removal lumen 616. The end face 606 of the conduit 611 has a concave surface 616 that extends at least partly across the respective ends of the fluid supply and fluid removal lumens 613, 615. This concave surface creates a cavity within the sheath between the concave surface 616 and the end of the sheath, to accommodate fluid flow F from the outlet of the fluid supply lumen 613 to the fluid removal lumen 615, ensuring that neither the outlet of the fluid supply lumen 613 of the inlet of the fluid removal lumen 615 is blocked by the sheath.

FIGS. 28 and 29 illustrate another embodiment conduit structure 711. In this embodiment, the internal wall 714 separating the fluid supply lumen 713 and the fluid removal lumen 715 terminates before the end of the conduit 716. This setting back of the dividing wall 714 provides a passage for fluid to flow F between the two lumens within the conduit 711, at a point spaced from the conduit end wall 716. This ensures that the passage between the lumens 713, 715 is maintained if the sleeve is sucked against or into the exposed end of the conduit 716 during use.

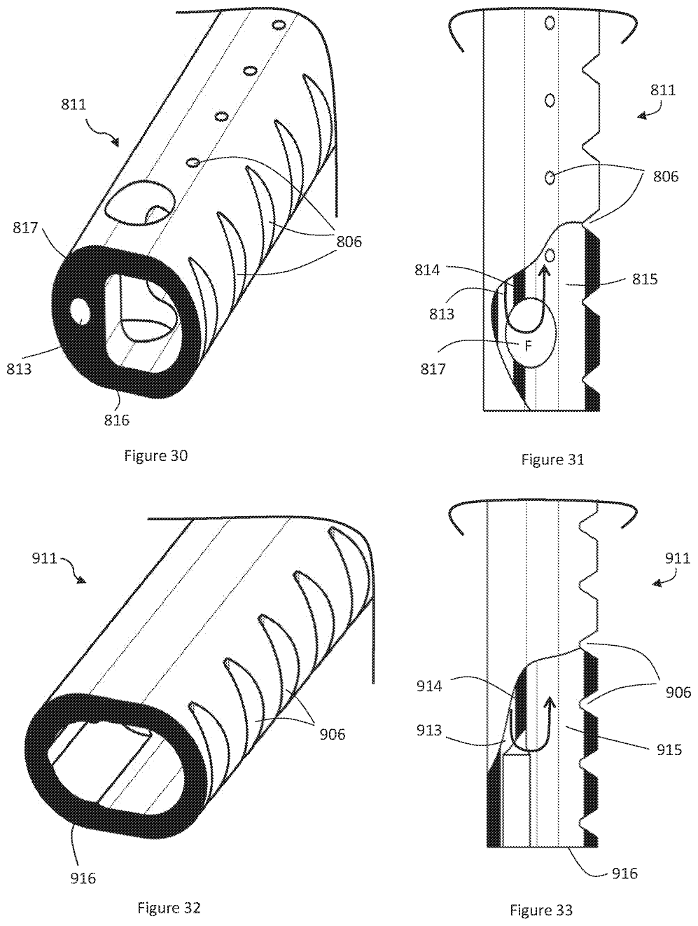

FIGS. 30 and 31 illustrate a further embodiment conduit structure 811. In this embodiment, apertures 806 along the fluid removal conduit are provided by both rows of apertures and a row of slits. The apertures 806 are elongate, slit-like apertures, V-shaped in profiles. A through hole 817 is provided through the conduit, perpendicular to the longitudinal direction of the conduit 811, at a point spaced from the conduit end 816. This through hole extends through the internal wall 814 separating the fluid supply lumen 813 creating a passage for fluid to flow F between the two lumens within the conduit 811, at a point spaced from the conduit end wall 816. This ensures that the passage between the lumens 813, 815 is maintained if the sleeve is sucked against or into the exposed end of the conduit 816 during use.

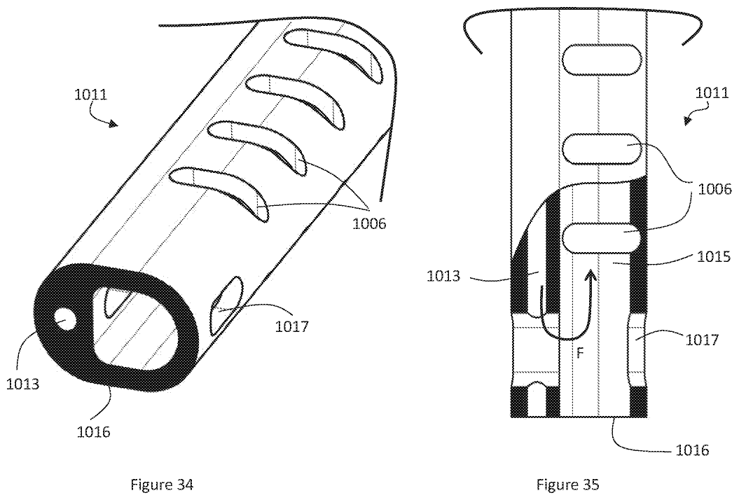

FIGS. 32 and 33 and FIGS. 34 and 35 illustrate two further embodiment conduits 911, 1011 illustrating variations of the previously described embodiment. The conduit of FIGS. 32 and 33 comprises a shortened internal wall 914, and a row of slit-like apertures along the fluid removal conduit 915. The conduit of FIGS. 34 and 35 comprises a transverse through hole 1017, and dual rows of slit-like apertures 1006. It will be appreciated that features may be selected from different ones of the embodiments described above and combined as desired to create further embodiments.

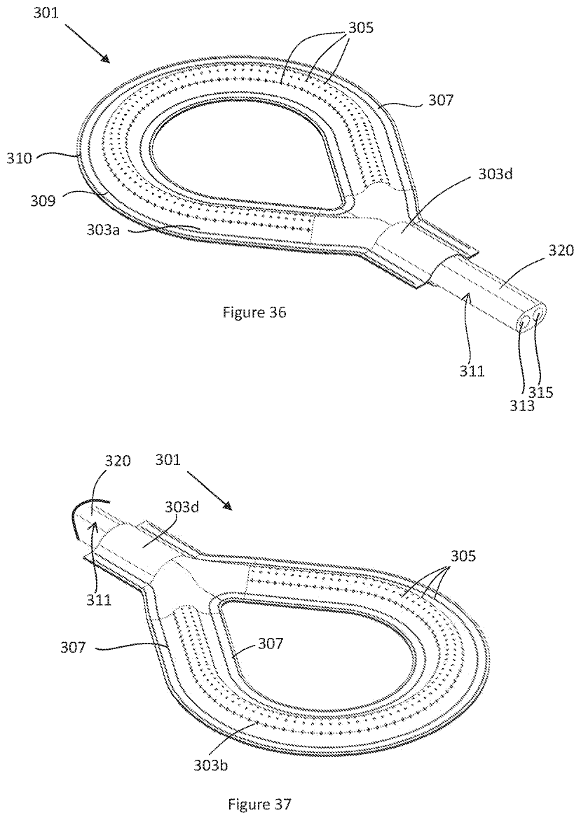

FIGS. 36 to 40 illustrate an alternative form device 301 that may be particularly suitable for large wounds. In some applications this embodiment device 301 may be placed along a plane of tissue, for example secured to a fascia of repaired muscle, or across a plane of repaired tissue, for example, incorporated into a repair across a layer of tissue such as the closure of the sub-cutaneous layer of tissue. In this embodiment, the fluid supply lumen 313 and the fluid removal lumen 315 are coaxial and form a loop.

The conduit structure 311 in this embodiment 301 comprises a coupling inlet/outlet component 320 of the device 301, which defines a portion of the fluid supply lumen 313 and an outlet portion of the fluid removal lumen 315. The component 320 is Y-shaped, with the lumens 313, 315 being generally parallel and side-by-side for a length through the component, then diverging at the end of the component 320 distal to the inlet/outlet ports. The lumens 313, 315 and have the same cross-sectional shape and size. In this example the lumens 313, 315 are circular in cross-section, but other shapes are envisaged.

The diverging ends of the coupling inlet/outlet component 320 are configured to fluidly couple to a structure 321 that forms the framework for the remaining length of the fluid removal lumen 315. Therefore, in this embodiment, the fluid supply lumen 313 extends only through the coupling component 320 (and optionally any upstream coupled component) and so is much shorter in length than the fluid removal lumen 315 which extends around the loop as well as through the coupling component 320.

The walls of both the fluid supply and fluid removal lumens 313, 315 are substantially fluid impervious through the coupling component 320 such that 100 percent of fluid supplied to the device 321 is supplied to the looped portion 321 of the conduit structure 311. The coupling component 320 may be a moulded component, for example, moulded from silicone.

If this spacing is too close, supplied fluid may bypass the looped portion 321 and thereby the majority of the treatment site and instead be drawn straight out of the device 301.

Any conduit structure defining an elongate channel may be suitable for use in the looped portion 321 of the conduit structure 311, for example, a truss-based or hollow extrusion type conduit structure. The structure 321 may comprise bioresorbable material that doesn't require removal from the wound site, or it may comprise a non-resorbable material such that the structure 321 will be removed once treatment is completed.

The looped structure advantageously enables the application of vacuum pressure to the centre portion of the device which applies vacuum pressure directly to the central area of the treatment site which is positioned the furthest away from the edges of the defect site (which has the highest likelihood of moving or remaining detached).

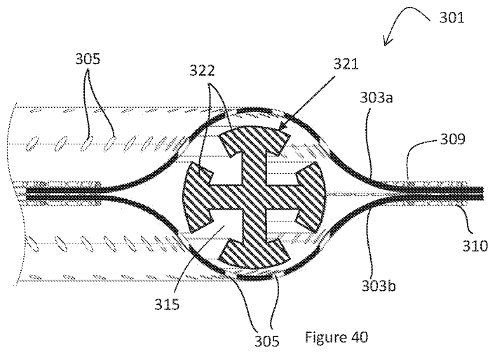

FIG. 40 illustrates one form of conduit structure 321 for use in the embodiment of FIGS. 36 to 39. The structure 321 is formed from a non-resorbable material such as silicone and comprises an extrusion having an X-shaped cross section The X-shape defines four flow paths along the fluid removal lumen 315. Curved flanges 322 on the ends of each cross member act to support the sheath 303 sufficiently to prevent the sheath being drawn into the fluid removal conduit upon the application of negative pressure. The flanges 322 define four elongate slit-like openings into the four respective passages to allow for the passage of supplied fluid out of the lumen and for the passage of wound fluids into the lumen 315.

In the device 301 of FIGS. 36-40, the sheath 303 encompassing the structure 321 comprises a top sheet 303a and a bottom sheet 303b, with a single flange 307 formed around the outer perimeter of the device and a second internal flange with an opening in the centre of the loop. Apertures 305 are provided in both the top and bottom sheath sheets 303.

The sheath forms an inlet portion 303d free of apertures that wraps over the Y-shaped coupling 320.

FIGS. 41 to 45 and 46 to 51 illustrate two alternative loop-type embodiments 1101, 1201. In these embodiments, the separate Y-shaped connector is omitted, and instead the conduit structures 1111, 1211 are manufactured as an integral component with a dual lumen portion 1111a, 1211a splitting at a junction into two separate single-lumen limbs 1111b, 1211b, 1111c, 1211c. Each limb 1111b, 1211b, 1111c, 1211c comprises a plurality of apertures 1106, 1206 for the exchange of fluids through the wall of the structure 1111, 1211.

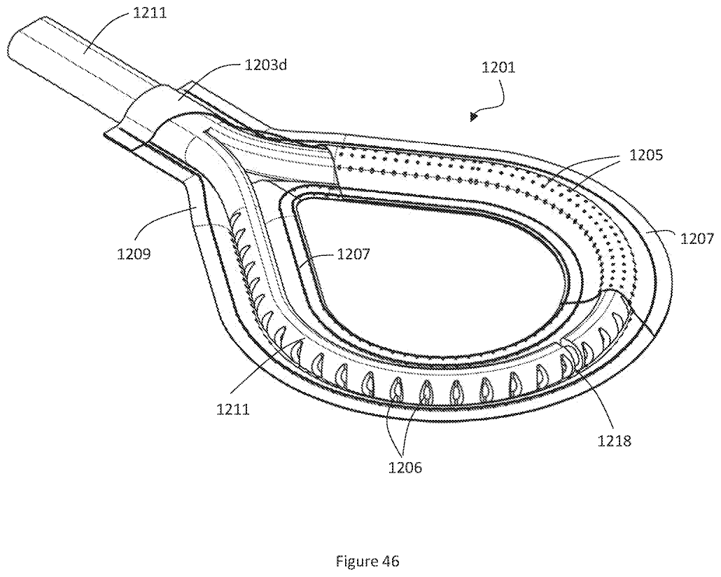

FIGS. 45 and 51 illustrate the respective conduit structures 1111, 1211 before assembly of the device 1101, 1201. To assemble the device, the ends of the conduit structure limbs 1111b, 1211b, 1111c, 1211c are butted together 1118, 1218 to be coaxial, forming a single continuous lumen through the device 1101, 1201. The sheath then holds the limbs in position when the two sheets are stitched together around the conduit structure. Alternatively, an additional sleeve of bioresorbable material may be tightly wrapped around the conduit limbs where they are butted together 1118, 1218 to firmly maintain the conduits in position. For those skilled in the art other means for maintaining these conduit limbs in position is envisaged.

The aperture arrangements 1106, 1206 on the conduit structures 1111, 1211 of these two embodiments 1101, 1201 are examples only, and many different aperture shapes and layouts will be possible. For each limb of the conduit structure, the wall of a first length of the conduit adjacent the Y-junction is free from apertures 1106, 1206 such that no fluid transfer into or from the lumen is possible over that length. These aperture-free portions of the limbs 1111b, 1211b, 1111c, 1211c are important to create a spacing S between the first point at which supplied fluid can migrate through the sheath 1103, 1203 to the wound site, and the first point at which fluid can be drawn from the wound site. If this spacing S is too close, supplied fluid may bypass the looped portion and thereby the majority of the treatment site and instead be drawn straight out of the device 1101, 1201.

As for the above embodiments, the sheath 1103, 1203 may comprise apertures, or may be free from apertures and rely on the porosity of the sheath for fluid transfer. In the embodiment 1101 shown in FIG. 41, the sheath is free from apertures and relies on the porosity of the sheath to facilitate fluid transfer across the sheath 1103. FIG. 59B illustrates flow AF across a processed layer of ECM material. The sheath 1203, in the embodiment of FIGS. 46 to 48 comprises multiple rows of apertures on both the top and bottom sheath sheets 1203a, 1203b.

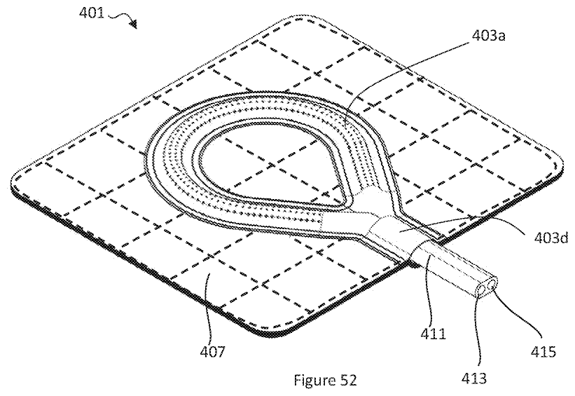

FIGS. 52-54 show a further embodiment 401, where the fluid drainage and supply device is incorporated with a multi-layer reinforced surgical mesh. The surgical mesh may be resorbable or non-resorbable. The surgical mesh forms the lower layer of the sheath 403b. A top sheet of material forms the top layer of the sheath 403a. Apertures 405 are provided on the top layer 403a of the sheath. This top layer 403a may cover the entire surgical mesh or may be shaped to only cover the conduit structure 411. The multilayer surgical mesh is reinforced using stitching 410 in a pattern that accommodates the conduit structure 411 and ensures the conduit structure 411 can be removed when treatment is concluded.

This embodiment 401 may have particular application for abdominal wall repair. If used, for example, in a complex hernia repair the sheath apertures 405 would typically face towards the skin and away from the abdominal cavity. This advantageously ensures the vacuum is applied to the separated tissues that lay above the device, ensuring effective fluid removal and the removal of dead space. The underside of the device 403b is free from apertures, which can aid in the healing of abdominal wall by preventing the application of vacuum pressure resulting in the underlying tissue adhering to the surgical mesh. While the apertures 405 the top sheath layer 403a act to improve the apposition of the separated subcutaneous tissues to the device 401 to diminish the clinical dead space that remains following the completion of the surgery.

In an alternative embodiment multiple conduits and/or upper sheath layers may be fixed to a single mesh.

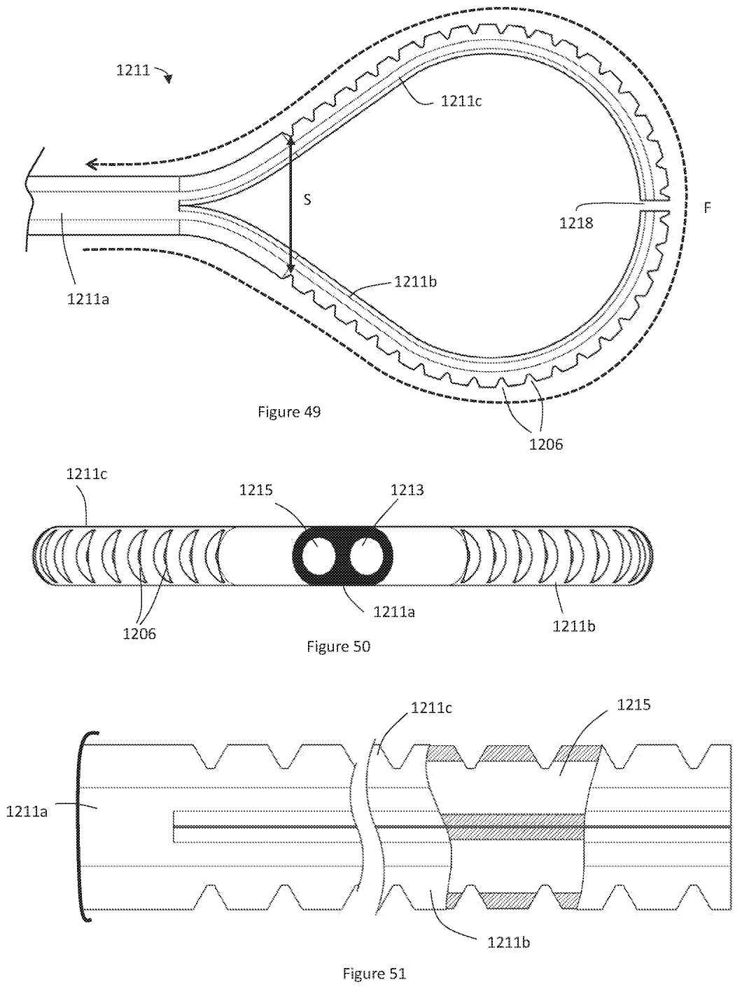

FIGS. 55 to 57 illustrate a similar embodiment device but having an alternative conduit structure 1311 that is substantially as described with respect to FIGS. 49-51. In this embodiment the top of the sheath 1303a has a series of apertures, where the underside of the sheath 403b is free of holes. The lower sheath 1303b of the device 1301 could be formed from one or more layers of polymeric material, for example, ECM, polymer, foam etc, for use as an implant or as a cover to achieve a vacuum seal over a wound.

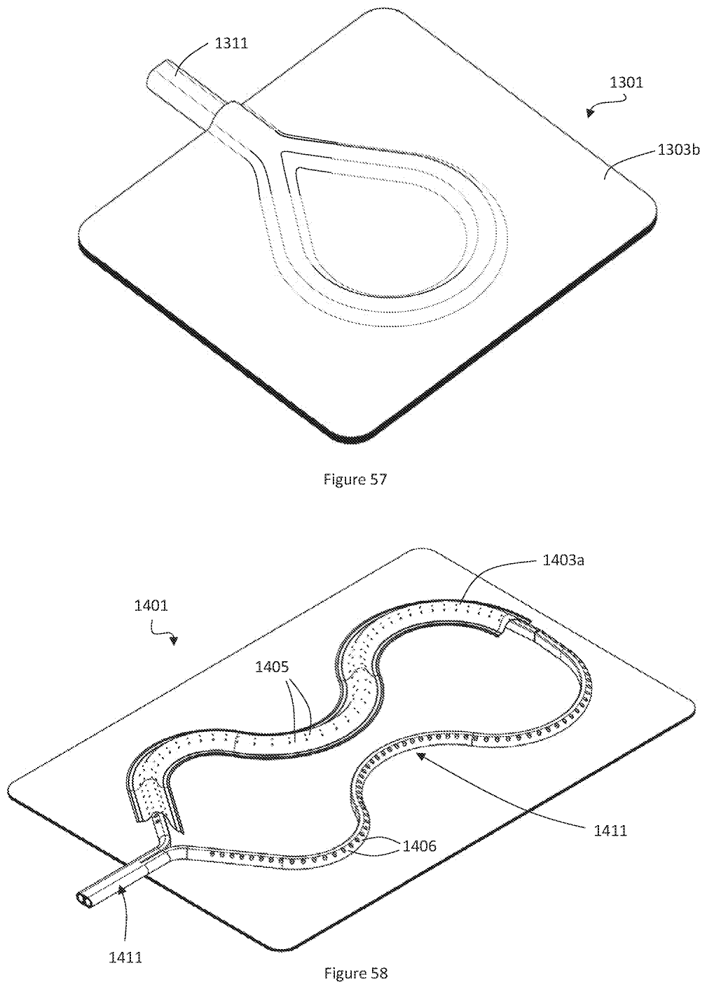

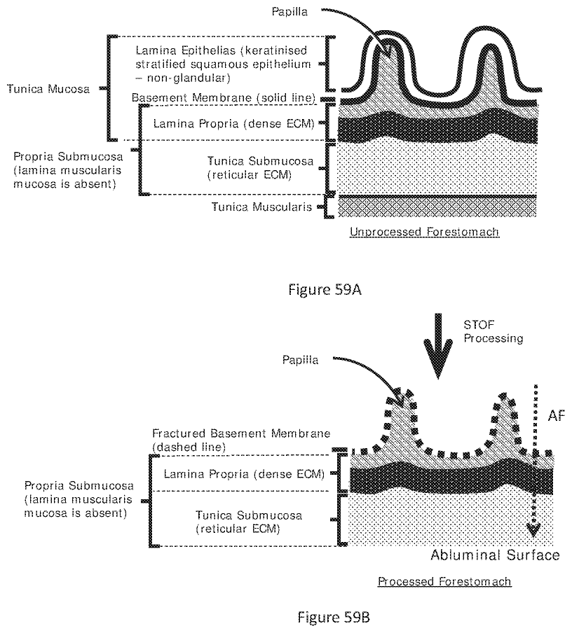

As illustrated by the exemplary embodiment 1401 in FIG. 58, the conduit structure 1411 incorporated into a multi-layer product or structure may follow any desired path. For devices that cover larger areas, snaking of the conduit structure, as illustrated in FIG. 58, may be desirable to increase the length of the fluid removal lumen and thereby to increase the area across which fluid and/or negative pressure is delivered.

Greater conduit coverage across the surface of the surgical mesh will increase the total force supplied to the treatment site, which improves the likelihood of achieving complete dead space closure and fluid removal and is likely to improve the clinical outcomes of the treatment.

For all embodiments, the devices may be engineered to provide a longer or shorter resorption time by adding additional layers of bioresorbable material to the sheath of the device, or by providing sheath layers that will be more quickly resorbed. Longer resorption time may be advantageous for sites which require prolonged periods of vacuum pressure, drainage or the delivery and removal of fluid. A prolonged ‘service lifetime’ of the device may also be obtained through the use of either non-absorbable suture material in truss-based conduit structures or long-lasting absorbable materials for the stitching on the seam features. The size of the device apertures can also be reduced in those situations where prolonged removal of fluid is favoured over the application of vacuum pressure to the surrounding tissue.

The device 1 . . . 1401 described herein is configured to allow the effective supply of fluid to and removal of fluid from a treatment site. Specifically, the fluid being supplied to and removed from the treatment site that is receiving a consistent vacuum pressure of between 60 mmHg to 150 mmHg.

The treatment site may be a space between surfaces of muscle tissue, connective tissue or skin tissue that have been separated during surgery or as a result of trauma and/or any site where soft tissue has been removed or repaired. The device may also be wholly contained within a layer of tissue, such as the sub-cutaneous layer or muscle layer, where the application of vacuum pressure and/or the delivery and removal of fluid is desired. Some examples include the abdominal wall after surgery, or the breast post-mastectomy or breast reconstruction. The treatment site may be the site of a seroma or hematoma, or maybe used as a prophylactic following surgical excision of tissue. Alternatively, the treatment site may be an open wound such as following trauma, injury or surgical excision of necrotic or infected tissue which can either be closed via an advancement of a tissue flap or sealed using an occlusive layer, such as a drape, to ensure a level of vacuum pressure can be sustained.

The treatment site may also be a site traversing across one or more layers of tissue, for example, across all or a portion of the subcutaneous tissue layer, from the interface with the underlying muscle layer to the connection with the dermal or epidermal layer of skin. One example may be a treatment site at which the flange of the device was anchored or affixed to a muscle fascia at one side, with the remaining device positioned within the subcutaneous layers of tissue during closure of a primary surgical incision, such as following a caesarean incision or a laparotomy.

The tabs or flanges 7 . . . 1407 of the device 1 . . . 1401 advantageously allow the device to be secured at the wound site by suturing the tabs or flanges to tissue in a position where the application of vacuum pressure, fluid removal, and/or targeted delivery of treatment fluids is most desired. This allows the targeted administration of vacuum pressure to areas of the treatment site that would most benefit from the obliteration of post-surgical dead space and removal of fluid, such as a site with extensive resection where a resultant tissue gape or mismatch will exist following primary closure of the surgical site, for example an internal tumour site or a donor site.

The ability to retain the device in place at the treatment site also helps to ensure the device will continue to function for a prolonged period of time once the patient starts to move. In other prior art devices, unwanted movement of the device within the treatment site can cause further internal trauma, prevent the previously separated tissue planes from being held back together by any administered vacuum, and can cause the conduit to move to a site where the movement of bone and/or muscle could cause the conduit to become blocked or pinched within the body.

A suitably shaped device may be selected, or for some embodiments, the length or shape of the device may be adjusted to best suit the wound site and the desired treatment areas. This may also include selecting or adjusting or shaping the device to avoid proximity to area where the application of negative pressure may be undesirable, for example, where it may be unsafe. Such sites may include areas of ligated vessels, exposed nerves, or other sensitive vessels.

The device 1 . . . 1401 is used as part of a system for delivering and draining fluid from the treatment site. The device conduit structure holds the two tissue surfaces spaced apart, thereby defining a channel into which fluid from the treatment site can drain or from which fluid can be delivered to the treatment site. The two tissue surfaces need to be held apart because they would otherwise collapse together, particularly under application of negative or reduced pressure (vacuum) to assist with fluid drainage.

A port in the form of an opening or a pair of openings at one end of the device 1 . . . 1401, allows for connection of the channel with a source of negative pressure or positive pressure. The port may be a dual lumen conduit and/or may be provided by the exposed open ends of the supply and removal conduits 13 . . . 1413, 15, . . . 1416. A fluid supply conduit is releasably coupled to the port to be in fluid communication with the fluid supply lumen, and a fluid removal conduit is coupled to be in fluid communication with the fluid removal lumen.

In some embodiments, the port may be coupled to an impermeable dressing located on the exterior surface of the patient's skin which provides an airtight hermetic seal around the incision of the skin and an alternative means to which a conduit is releasably coupled to the dressing. In other embodiments, the port could be provided by a connector that interfaces with the conduit structure and an external device.

A reservoir is located external to the body of the patient, and arranged in fluid communication with the fluid removal lumen for receiving fluid from the device. The source of pressure may be capable of delivering negative pressure to the device so that fluid is drained from the treatment site into the device and transferred through the conduit to the reservoir and/or so that a treatment fluid is drawn through the conduit into the device and delivered to the treatment site, or may be capable of delivering positive pressure to the device so that fluid in the reservoir is transferred through the conduit into the device and to the treatment site. The treatment fluid may be a liquid or a gas, for example may include filtered air or other mixed phase fluids such as vapour or humidified air. In embodiments where a treatment liquid is introduced, a further reservoir containing or holding a treatment fluid may be coupled to the fluid supply lumen for delivering the treatment fluid to the device.

The source of pressure will typically be a pump for pumping fluid from the reservoir into the device for delivery to the treatment site or a vacuum pump for applying negative pressure to drain fluid from the treatment site. The system operates to substantially maintain the negative pressure of the treatment site during the introduction of filtered air and/or other treatment fluids. The pump may be manually operated, for example using a squeeze bulb, or may be electronically controlled for more precise delivery of fluid to the site. One particularly suitable pump is described in U.S. application 63/117,995, incorporated herein by reference.

In a system where fluid is being delivered to the treatment site, the fluid to be delivered may contain one or more nutrients, ‘flowable fluids’ such as Thixotropic gels or highly viscous fluids that can still be transported via a conduit, cell-suspensions therapeutic agents for promoting wound healing. The fluid may comprise flowable gels derived from ECM, hyaluronic acid, growth factors to aid healing, to antimicrobial drugs for the treatment of infection, analgesic drugs such as fentanyl or morphine for pain relief and anti-inflammatory drugs such as ketorolac or diclofenac, for example, although other fluids are envisaged and will be apparent to a skilled person.

In some alternative embodiments the device could be operably connected to one or more other devices, implanted at different respective sites for treating the respective sites with the same pressure source.

### Animal Studies

### Test 1

Prior to commencement of the animal study, a bench test was carried out in which the device 1101 illustrated in FIG. 41 to 45 was placed within a sealed polyurethane bag and connected to the pump device described in our U.S. application 63/117,995. Blood from a blood bag was mixed with a coagulant to simulate a slow clot and instilled into the device 1101 to assess the ability of the implant to maintain the vacuum pressure at a treatment site when the clotted fluid is being removed by the connected pump via the repeated delivery of filtered air to the implant.

The test setup confirmed that an implant device such as the device 1101 of FIGS. 41 to 45, without sheath apertures, can effectively deliver the vacuum pressure to the treatment site.

The internal conduit structure of this implant was approximately 260 mm long and constructed using two Ø3.2 mm mandrels where a Ø450 μm (410-450 μm) monofilament polypropylene suture was used to construct a truss with a pitch length of 2.5 mm in between the nodes which proves an internal lumen area of ˜16 mm². The device 1111 was sewn with two runs of Ø125 μm (100-149 μm) PGA multifilament stitching along the seam 1109 on both sides of conduit structure to secure the upper and lower sheath sheets 1103a, 1103b over the conduit structure 1111.

As illustrated in FIGS. 59A and 59B, fluid flow rates through ECM sheets is higher in one direction compared to the other direction. In embodiments tested, the ECM sheets were arranged with the papilla (luminal) surface of the ECM facing outward, away from the internal conduit structure 1111.

The device 1101 was placed into a sheep weighing 90 kg where the entire latissimus dorsi muscle was removed (weighing 195 g). The conduit structure 1111 of the device was coupled to a dual lumen silicone tube which had a fluid supply lumen size of 1.65 mm² (Ø1.45 mm) and a fluid removal lumen size of ˜9 mm² (equivalent diameter of Ø3.39 mm) via a push fit connector, which had an internal conduit area of 9.65 mm².

The multi-lumen silicone tube was in turn connected to an external battery powered vacuum device which was targeting the maintenance of the vacuum pressure measured along the fluid supply lumen of between 60 mmHg-115 mmHg, where the pump was configured to limit the vacuum being supplied along the fluid removal lumen to a maximum of 150 mmHg. The pump was configured to ensure that this level of vacuum pressure was maintained during the introduction of filtered ambient air which was controlled via a valve that was programmed on a cycled of 14 seconds open and 20 seconds closed. The valve cycle was operating in a continuous cycle until the pressure measured at along the fluid supply lumen reached a 60 mmHg vacuum pressure threshold, at which point the system ceases to operate the valve.

The vacuum pressure measured at far end of the implant/along the fluid supply lumen of the tube was found to sustain the target 60-115 mmHg vacuum pressure for a period of approximately 5½ days following surgery where a total amount of 349 g of exudate was removed during this time.

Upon euthanasia of the study animal it was found that a large seroma had formed over the top of the implant device, concluding that while the device was effective for removing fluid for the entire duration of treatment it was not effective at managing the post-surgical dead space created by the complete resection of the latissimus dorsi muscle.

### Test 2

The animal study described above for test 1 above was repeated using an implant device 1201 shown in FIG. 46 to 51, with the Ø0.5 mm aperture features in the sheath. In this test the only other variable modified was the addition of sterile saline supplied via the fluid supply lumen at periodic intervals over the first 3 days of treatment post-surgery, to assist in purging the device 1201 of any clotting factors such as fibrin/fibrinogen or other blood components. The sterile saline was delivered to the treatment site using a manual means to draw sterile saline through the implant by utilising the vacuum pressure being maintained within the implant device, which maintains the vacuum pressure at the treatment site throughout the introduction of the saline.

In this test a total of 345 g of saline was added, comprising of 92 g on the day following surgery (t=0), 142 g two days following surgery (t=2) and 111 g three days (t=3) following surgery. The vacuum pressure measured along the fluid supply lumen was found to fall below the 60 mmHg lower vacuum pressure threshold at approximately 2½ days following surgery at which point the opening and closing of the air valve ceases to function, with the system targeting a constant vacuum pressure level within the system at a single pressure level.

A sheep weighing 87 kg was used for the study with the removed latissimus dorsi muscle weighing 116 grams. A total of 800 grams of exudate was removed from the animal over a period of 5 days following surgery.

Upon euthanasia of the study animal it was found that a clearly defined line of highly opposed tissue crossing over the middle of the device with approximately 50-70% of the device well integrated with the surrounding tissue. The region of the defect site laying in closer proximity to the ulna (cranial end of the animal), accounting for approximately 30-50% of the device, was found to have a seroma, with the opposing side of the defect area extending towards the rear (caudal end of the animal) appearing to be completely healed with well opposed tissue.

The gross observations from this euthanasia found there to be significantly improved clinical outcomes for dead space management following treatment when compared to the previous device.

### Test 3

A further animal study was performed to compare the treatment outcomes of a linear device similar to the device 101 shown in FIGS. 16-21 to an existing prior art closed wound drainage device (control device).

The closed wound drainage (control) device was a Cardinal Health 3-spring mechanically powered closed wound drainage device with a perforated 15 Fr (Ø5 mm O.D tube-Ø3 mm ID tube) sized PVC drain (part number SU130-403D). These reservoirs are known to deliver approximately 70 mmHg of vacuum pressure when fully primed.

The treatment device was approximately ˜100 mm in length where the central conduit device was a multi-lumen silicone tube with a cross sectional shape shown in FIG. 21. The fluid supply lumen 113 is Ø1.4 mm and the fluid removal lumen 111 is approximately 18 mm² in area. The conduit was fabricated as shown in the embodiment in FIGS. 28 and 29 with a series of 3×Ø3 mm sized cut apertures 106 positioned around the top, side, and bottom face of the fluid removal lumen, which are distributed along the length of the conduit that is positioned within the sleeve. The internal wall between the two lumens 113, 115 was back cut in the manner illustrated in FIGS. 28 and 29 to ensure the path between the fluid supply lumen and the opening of the fluid removal lumen is preserved when the device is under vacuum.

The implant device was fabricated with two sheets of ECM material which were sewn using Ø125 μm (100-149 μm) PGA multifilament stitching along the seam on both sides to secure the sleeve with the papilla (luminal surface) side of the device facing inwards towards the conduit. Both sides of the sleeve device contained a series of Ø0.5 mm diameter apertures along the length of the device.

The external vacuum pump device connected to this implant was configured to open the fluid supply valve for 14 Seconds with a closed duration of 2 minutes which introduces filtered air into the conduit via the fluid supply lumen with the system maintained at a vacuum pressure level of 80 mmHg during the instillation of filtered air. Once the air valve closes to return the device to a second equilibrium pressure of 100 mmHg. This cycle continues to operate until the point at which the pressure being measured along the fluid supply lumen of the device drops to 60 mmHg or below, at which point the opening cycle of the valve ceases to operate and the system targets the constant delivery of 100 mmHg along the fluid removal lumen of the device. The system is programmed to ensure the vacuum pressure level along the fluid removal conduit does not exceed 150 mmHg as a safety mechanism.

An ovine bi-lateral external abdominal oblique dead space seroma model was used to compare the two devices. In this study a sheep weighing 58.5 kg was used with one side receiving the treatment device and the other receiving the closed wound drainage (control) device.

The closed wound drainage device defect site was created by excising 18 grams of external abdominal oblique muscle from an undermined area above the muscle to create a resultant defect area of approximately 38 cm². The drainage catheter was placed into the wound at the lowermost aspect of the wound with the tube also ported at the lowermost aspect of the wound. The tube was routed alongside the external surface of the sheep and was connected to a spring reservoir located within an equipment harness located on the back of the animal, which provides the vacuum pressure to the catheter. The reservoir was primed and connected to the treatment device once the wound was closed to administer the 70 mmHg of vacuum pressure to the perforated catheter.

The treatment device was placed within a defect that was created on the opposing side of the animal. A defect site of ˜82 cm² in area was created by excising 19.5 grams of external abdominal oblique muscle from an undermined area above the muscle. The implant device was positioned along the longitudinal axis of the defect site which extended vertically across a defect site on an angle. The implant device was secured to the treatment site using a series of passed sutures that were tied off to affix the implant in place. Once the treatment site was closed the implant device was connected to the externally mounted vacuum pump device to function as programmed.

For this animal study the treatment duration for both the treatment and control devices were set to run for 14 days following surgery. The manually powered closed wound drainage (control) device was primed each day to ensure the continual application of negative pressure where the amount of wound exudate collected by both devices was also weighed and recorded each day.

An ultrasound assessment was performed at days 7 and 14 post-surgery to assess the size of any seroma forming at the defect site for both devices. The volume of any seroma measured at the defect site was calculated using the formula to determine the volume of an ellipsoid.

Both the treatment and control devices were removed at 14 day post-surgery using a force gauge to determine the removal force. The animal was euthanised at 28 day post-surgery to perform a gross assessment of the defect site for both animals.

The results from the study are shown in the table below;

| Total Collected | | Seroma Volume | | Seroma Volume | | MAX Tube | |
|---|---|---|---|---|---|---|---|
| Fluid after 7 | | on Day 7 using | | on Day 14 using | | Removal | |
| Days (mL) | | Ultrasound (mL) | | Ultrasound (mL) | | Force (N) | |
| Treat- | Con- | Treat- | Con- | Treat- | Con- | Treat- | Con- |
| ment | trol | ment | trol | ment | trol | ment | trol |
| 153 | 55 | 0 | 0 | 0 | 521 | 5.46 | 8.06 |

Following 7 days of treatment the closed wound drainage (control) device was found to collect a total of 55 mL's of wound exudate where the treatment device was found to collect 153 mL's of exudate. There was no seroma present at either the 7 day or 14-day post-surgery timepoint for the treatment device, with the closed wound drainage (control) device found to have zero seroma at the 7-day time point and a large ˜521 mL (cm³) seroma at the defect set following 14 days post-surgery.

The force required to remove the conduit of the treatment device from the animal at the 14-day post-surgery treatment end point was recorded at a maximum of 5.5 N with the PVC tube from the closed wound drainage (control) device requiring a maximum of 8.1 N.

A gross examination of the defect sites for both the treatment and closed wound drainage (control) devices was performed at 28 days post-surgery. The defect site with the treatment device was found to be completely integrated with no signs of any seroma or wound fluid at the defect site. The defect site with the closed wound drainage (control) device was found to have a large seroma consistent with the ultrasound findings at the 14-day post-surgical timepoint, with virtually zero signs of any integration of the separated tissue planes of the defect site.

The results from this observation confirmed that the treatment device provided complete dead space closure of the defect site following surgery.

Although the invention has been described by way of example, it should be appreciated that variations and modifications may be made without departing from the scope of the invention as defined in the claims. The embodiments described herein are provided to exemplify alternatives for various features of the device. It will be appreciated that many permutations of these features are possible to create other embodiments within the scope of the invention claimed. That is, features from one embodiment may be combined with features of another embodiment to create a new embodiment that remains within the scope of the present invention. Furthermore, where known equivalents exist to specific features, such equivalents are incorporated as if specifically referred in this specification.

The device described herein may advantageously be customised to adjust the duration for which the device is functional in-situ for any given application. For example, by adjusting channel size, wall thicknesses, or the thickness or density of truss members, or the number and type of bracing members.

### System

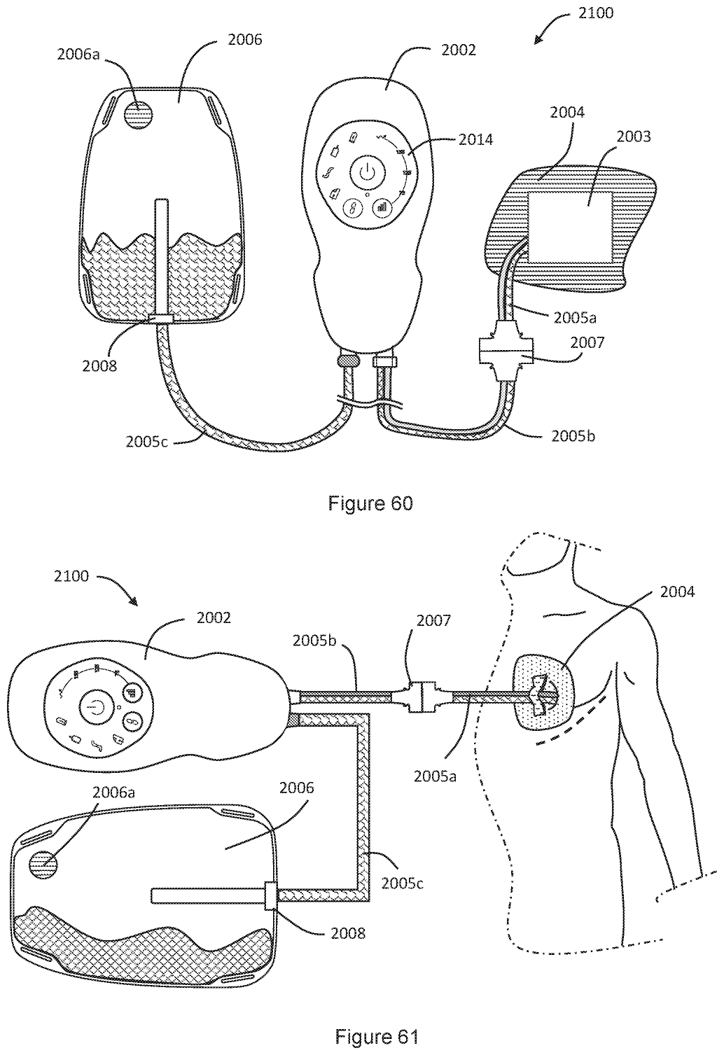

FIGS. 60 to 64 show exemplary embodiments of negative pressure treatment systems 2100, 2200, 2300 (herein treatment systems) for the removal of fluid from a wound or for supplying treatment fluid to a wound 2004 and removing fluid from a wound using a wound treatment device 2003. The wound treatment device 2003 of the system may be any one of the devices 1 . . . 1401 described herein.

In relation to the exemplary embodiment systems, like reference numbers are used for different embodiments to indicate like features.

Referring to FIG. 60, at a general level the treatment system 2100 comprises a wound treatment device 2003 to be located at a wound treatment site 2004 (‘wound’), a vacuum pressure unit 2002 comprising a vacuum pump assembly for applying negative pressure to the wound 2004 via the treatment device 2003, and a fluid collection reservoir 2006 for collecting fluid returned from the wound 2004. FIG. 61 illustrates an internal wound site located at a chest area of a patient; however, the system may be used to treat internal wounds located at other sites for example to treat an abdominal wound.

The vacuum pressure unit (or vacuum unit) 2002 is configured to position the pump assembly 2015 upstream of the fluid collection reservoir 2006 and downstream of the wound treatment device 2003. The wound treatment device 2003 may comprise a topically applied wound dressing, an implanted treatment device or a combination of both in a coupled configuration. The fluid collection reservoir 2006 is configured to include one or more air permeable filters or vents 2006a to maintain the fluid collection reservoir 2006 and connected conduit 2005c at an ambient pressure level.

The vacuum unit 2002 fluidly couples to the wound treatment device 2003 via at least one conduit. The conduit from the vacuum unit 2002 to the wound treatment device 2003 may comprise a two-part conduit, with a first conduit 2005b extending from the vacuum unit 2002, and a second conduit 2005a extending from the wound treatment device 2003. The second conduit may be part of the wound treatment device 2003 or may be connected to the treatment device 2003 by a connector (not shown). A connector 2007 is provided to fluidly couple the first and second conduits 2005a, 2005b. Alternatively, a continuous conduit may extend between the vacuum unit 2002 and the treatment device 2003.

The connector 2007 may comprise a one-way valve oriented to allow fluid flow in a direction from the wound 2004 towards the vacuum unit 2002 and prevent a backflow of fluid from the pump to the wound. In alternative embodiments, a one-way valve may instead be provided within the vacuum unit 2002, elsewhere on the conduit 2005a, 2005b, or as part of the treatment device 2003. In a further alternative, the treatment system 2100 may be without a one-way valve between the treatment device 2003 and the vacuum unit.

In some embodiments, the conduit(s) between the vacuum unit 2002 and the treatment device 2003 may comprise a dual lumen conduit with a primary lumen for the passage of fluid flowing from the wound to the pump assembly 2015, and a secondary lumen. The secondary lumen may allow for measurement of pressure at the wound site. The secondary lumen provides for the delivery of air and/or treatment fluids to the wound 2004. However, in alternative embodiments multiple conduit(s) may be provided between the vacuum unit 2002 and the treatment device 2003 each with a single lumen.

A further conduit 2005c is provided between the vacuum unit 2002 and the reservoir 2006 to fluidly couple the pump assembly 2015 to the reservoir 2006. A connector 2008 may be provided to fluidly couple the conduit 2005c to the reservoir 2006.

In preferred embodiments, the vacuum unit 2002 is a portable hand-held unit. The vacuum unit 2002 may be a single use unit that is intended to be used for a single patient. In an alternative embodiment the vacuum unit 2002 could be configured for multi-patient use. The vacuum unit 2002 comprises a (plastic) shell or enclosure to house the pump assembly 2015 and other components. The vacuum unit 2002 comprises a user interface 2014 for operating the vacuum unit 2002. The user interface may include controls to turn the pump assembly 2015 of the system 2100 on and off, and may allow an operator to control parameters of a pressure treatment being applied to the wound 2004 such as the level of vacuum pressure being applied or the length, size and frequency of pressure oscillations between upper and lower set points.

In alternative embodiments the user interface 2014 may also include controls to remotely connect a monitoring device to the vacuum unit to enable the transmission of data to an operator or user of the system to aid in the monitoring of treatment.

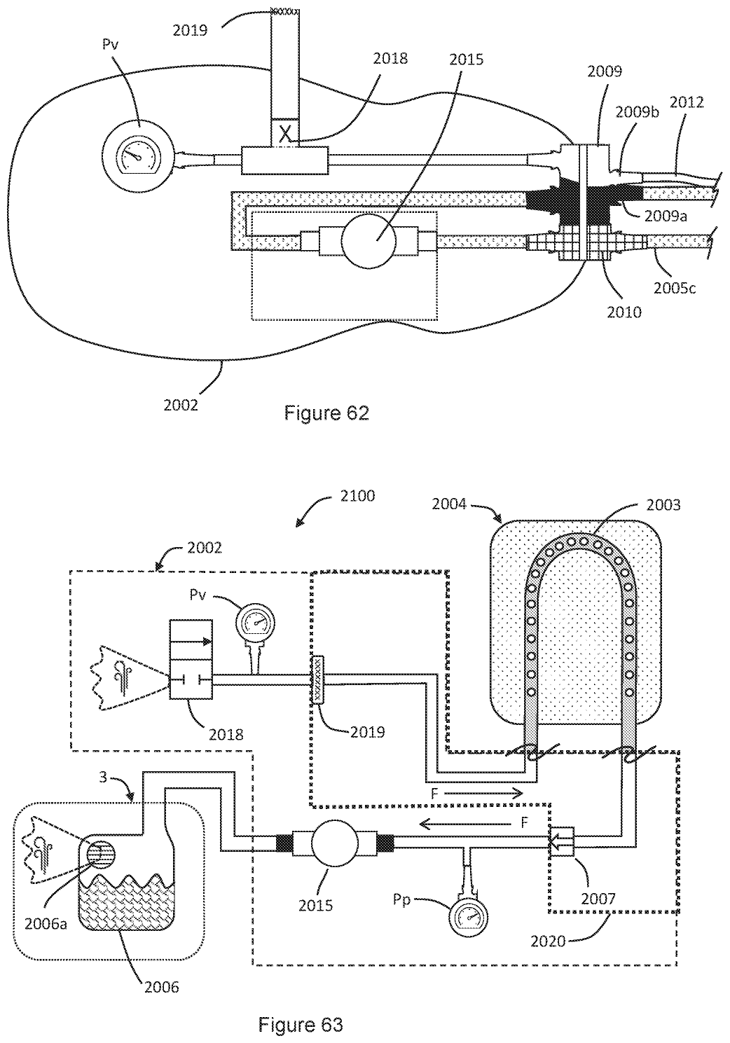

Referring now to FIG. 62 together with FIG. 60, the vacuum unit 2002 comprises a housing or enclosure that houses a vacuum pump assembly 2015 described in more detail below, batteries or other power supply, a vacuum unit connector 2009 in fluid communication with the conduit(s) 2005b, 2005a to deliver and receive fluid from the wound treatment site 2004, and a vacuum unit outlet connector 2010 in fluid communication with the conduit 2005c to the reservoir 2006, for the fluid flow from the pump assembly 2015 to the reservoir 2006. The connectors 2009, 2010 are configured to couple with ends of respective conduits 2005b, 5c and may be of any suitable form, for example, they may comprise luer-type connectors.

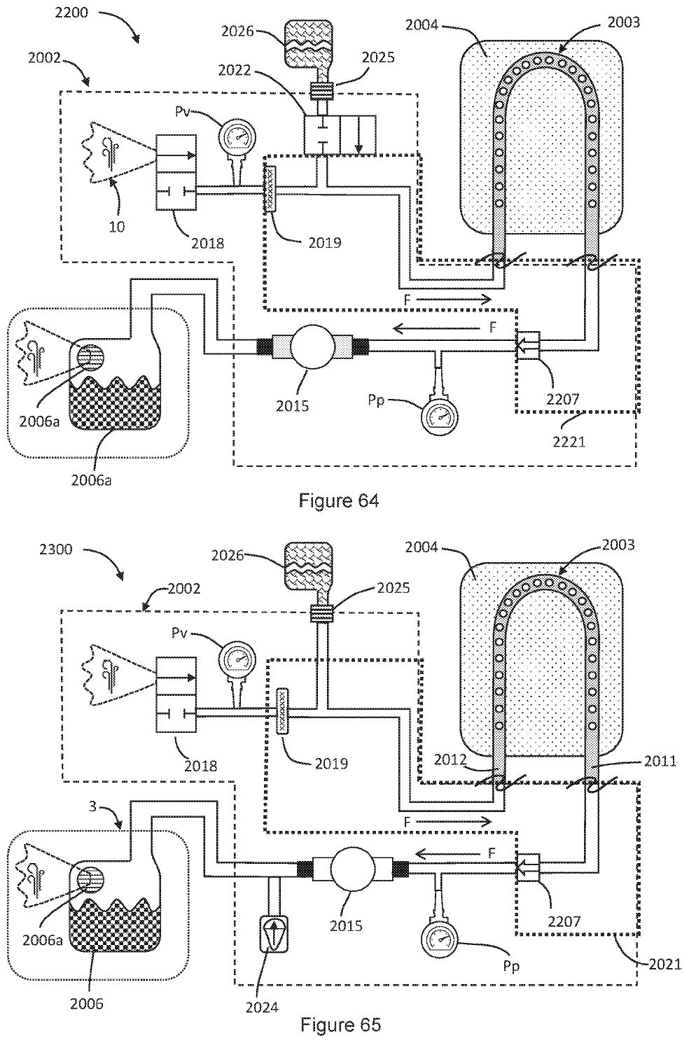

In one embodiment the vacuum unit connector 2009 may comprise two one-way valves such that a one-way valve within the secondary connector 2009b is oriented to allow the flow of fluids from an upstream source, such as ambient air that has been passed through a sterile filter (filter 2019 in FIGS. 64 and 65) or from a treatment fluid reservoir (reservoir 2026 in FIGS. 64 and 65), to the wound 2004. The corresponding one-way valve within the primary connector 2009a is oriented to allow the flow of fluid in a direction from the wound 2004 towards the vacuum unit 2002. In some embodiments the one-way valves within the primary 2009a and secondary 2009b connector may be configured to be closed when the vacuum unit connector 2009 is disconnected from the vacuum unit 2002. These valves are then subsequently opened to allow the passage of fluids when the vacuum unit connected 2009 re-connected to the vacuum unit 2002. Examples of known prior art connectors that possess such features include needle-free or needless connectors for use within IV applications, such as the BD® MaxPlus™ needle-free connectors, which only allow a passage of fluid once engaged with an appropriate luer-lock connector.

The conduit 2005b for fluid flow into and out of the vacuum unit connector 2009 is a dual lumen conduit with a primary lumen 2011 and a secondary lumen 2012. The connector 2009 includes a primary connector 2009a providing a fluid inlet to connect to the primary lumen 2011, and a secondary connector 2009b providing a fluid outlet to connect to the secondary lumen 2012 while keeping the flow from these lumens separated. The larger primary lumen 2011 allows the passage of fluid flowing from the wound, through the primary connector, to the vacuum pump assembly 2015. The secondary or supply connector 2009b may be separate from the primary or removal connector 2009a.

The primary and secondary lumens 2011, 2012 are preferably provided as adjacent passages in a single body/conduit along most of their length. However, adjacent the vacuum unit 2002 and/or adjacent the wound treatment device 2003, the dual lumen conduit 2005a, 2005b may be split or separated into two separate limbs or conduits, a supply conduit comprising the secondary lumen 2012 and a removal or exudate conduit comprising the primary lumen 2011, for ease of coupling to the vacuum unit 2002 and/or to allow the supply conduit to enter the wound or wound treatment device 2003 at a different location to the removal conduit. The primary or removal conduit and lumen may be referred to interchangeably and referenced by reference numeral 2011 and the secondary or supply conduit and lumen may be referred to interchangeably and referenced by reference numeral 2012.

The supply conduit 2012 is in fluid communication with a pressure sensor Pv to allow for measurement of pressure on an upstream side of the wound treatment device 2003.

The vacuum unit 2002 comprises an air inlet valve 2018 in fluid communication with the supply conduit 2012. The air inlet valve 2018 is controlled in a manner to introduce air into the treatment system 2100 to assist with lifting fluid from the wound site 2004, as described in more detail below.

As shown in FIG. 62, the air inlet valve 2018 may have an inlet to draw ambient air to the system from outside the vacuum unit 2002 enclosure. Alternatively, the inlet for the air inlet valve may take air from inside the vacuum unit housing/enclosure.

A sterile filter 2019 is provided to prevent the ingress of bioburden and non-sterile air into the system 2100 and wound site 2004. In FIG. 62, the filter 2019 is provided on an inlet of the air inlet valve 2018, however a filter may be placed at another location between the air inlet valve 2018 and the vacuum unit fluid supply connector 2009b, or between the air inlet valve 2018 and the wound site 2004.

FIG. 63 illustrates the treatment system 2100 schematically in more detail. The boundary or outer enclosure of the vacuum unit 2002 is illustrated by the dashed line in FIG. 63. On an upstream side of the treatment device 2003 the vacuum unit 2002 comprises the air inlet valve 2018, optionally the pressure sensor Pv and the sterile filter 2019, and on a downstream side of the treatment device 2003 the vacuum unit 2002 comprises the pump assembly 2015 and optionally a pressure sensor Pp between the pump assembly 2015 and treatment device 2003. The vacuum unit 2002 may also comprise a connection manifold 2020 providing a connection interface between the conduit 2005a, 2005b to the treatment device 2003 and the vacuum unit 2002. The connection manifold 2020 is illustrated by the dotted line in FIG. 63 and replaces connector 2009 shown in FIG. 62.

In the embodiment system 2300 of FIG. 65 the vacuum unit 2002 additionally includes a colour sensor 2024 that is electronically connected to the vacuum unit controller 2017. In this embodiment 2300, the colour sensor 2024 is positioned along the fluid flow path positioned between the outlet of the pump 2015 and the outlet connector 2010. However, the colour sensor could alternatively be positioned along the fluid pathway in any suitable position upstream of the inlet of the pump 2015.

The colour sensor 2024 may be beneficial to detect a colour change of wound exudate fluid flowing through the system from the treatment device 2003 at the wound site 2004. For example, a natural change in colour from a first blood rich wound exudate immediately following surgery, to a pink colour of serosanguinous drainage (blood and serum), and/or to a clear serous (serum only) drainage. This operation of the colour sensor 2024 may be enhanced by the supply of filtered air from upstream of the treatment device 2003. The filtered air displaces the fluid for a short to time frame to produce a readable sample of fluid within that short time frame, similar to that of a direct aspiration of fluid from the treatment site 2004 via a needle.

The inclusion of a colour sensor within various embodiment systems that supply treatment fluid to, and remove treatment fluid from, the wound may offer further benefits. For example, the colour sensor 2024 could be configured to detect the passage of treatment fluid being supplied from the treatment fluid reservoir 2026 and passing through the upstream fluid pathway, removal conduit 2011, wound treatment device 3 and supply conduit 2012, to the vacuum unit 2002 denoting the complete saturation of treatment fluid through the connected system. In other embodiments the treatment fluid could be combined with a colour based indicator for the detection of changes at the wound in response to the presence of infection, biofilm or other wound based pathologies.

FIGS. 64 and 65 illustrate further embodiment treatment systems 2200, 2300 for supplying fluid to and removing fluid from a wound. The embodiments of FIGS. 64 and 65 include the same or similar features of the system 2100 described above with reference to FIGS. 60 to 63, however are additionally configured to provide a treatment fluid to the wound treatment device 2003.

With reference to FIGS. 64 and 65, the vacuum unit 2002 may comprise one or more ports 2025 to receive therapeutic fluids for delivery to the wound site. The port 2025 is preferably configured to be nominally closed to the passage of liquids when disconnected from the treatment fluid reservoir 2026 which subsequently opens when engaged with a luer connector. The B. Braun Medical® CARESITE™ needless connector provides an example of such a port.

A therapeutic agent in the form of a treatment fluid may be selectively delivered to the wound treatment device 2003 via the supply conduit 2012. A fluid source or treatment fluid reservoir 2026 may be coupled to the fluid port 2025 of the vacuum unit 2002, for example via a conduit or connection to an intravenous (IV) fluid giving set such as a Baxter® EMC 9608 Admin Set, B. Braun Medical® Single Chamber IV Infusion Set or similar sterile IV infusion therapy set. The treatment fluid reservoir is preferably at atmospheric pressure whilst connected to the treatment system. This can be achieved by using a non-vented IV infusion therapy set in combination with a flexible fluid bag such as Baxter® Sodium Lactate (Hartmanns or compound sodium lactate) IV Bag or similar, or it may also be achieved by connecting a vented IV infusion therapy set to a rigid or semi-rigid container of treatment fluid, such as Prontosan® Wound Irrigation Solution by B. Braun Medical®.

Example therapeutic fluids include, but are not limited to, compound sodium lactate, physiological saline (0.9% NaCL—Sodium Chloride) and 0.45% normal saline (0.45NaCL). Antimicrobial agents and solutions could also be applied for the treatment of infections and may contain agents such as polyhexanide (PHMB), silver nitrate, hypochlorous acid (HOCI), sodium hypochloride, betaine, sodium hypochlorite, super-oxidized water with neutral pH or any other antimicrobial wound irrigation solutions.

Other treatment fluids may also include cell-suspensions and cell-based fluids for promoting wound healing. The fluid to be delivered may contain one or more nutrients, ‘flowable fluids’ such as Thixotropic gels or highly viscous fluids that can still be transported via a conduit, cell-suspensions therapeutic agents for promoting wound healing. The fluid may comprise flowable gels derived from ECM, hyaluronic acid, growth factors to aid healing, to antimicrobial drugs for the treatment of infection, analgesic drugs such as fentanyl or morphine for pain relief and anti-inflammatory drugs such as ketorolac or diclofenac, for example, although other fluids are envisaged and will be apparent to a skilled person.

Instillation of autologous or allogenic cell-based therapies containing either platelet rich plasma, stem cells, stromal cells, keratinocytes, lymphocytes, bone marrow aspirate, serum and dendritic cells could aid in the repair and healing of wounds.

The instillation of chemotherapeutic drugs could also aid in the localised treatment of cancerous cells that may not be operable, or could be used as an overall treatment plan following excision of cancerous tissue.

With reference to the embodiment 2200 of FIG. 64, a treatment fluid inlet valve 2022 is selectively operable to allow fluid to flow from the treatment fluid reservoir 2026 and into the supply conduit 2012 leading to the wound. The reservoir of fluid is at atmospheric pressure. When the treatment fluid inlet valve 2022 is selectively opened, negative pressure from the pump assembly 2015 applied to the wound 2004 via the removal conduit 2011 acts to draw fluid from the treatment fluid reservoir 2026 towards the dressing or wound treatment device 2003. Upon activation of the treatment fluid inlet valve 2022 the controller (not shown in this figure) within the vacuum unit 2002 detects a subsequent drop in the vacuum pressure level at the Pv and/or Pp pressure sensor(s) and activates the pump assembly 2015 to maintain the vacuum pressure at a target level of vacuum pressure. A control algorithm is described in more detail below. In the illustrated embodiment, the air inlet valve 2018 and sterile filter 2019 is provided upstream of the therapeutic fluid valve 2022.

In the embodiment 2300 of FIG. 65, the system is without a treatment fluid inlet valve 2022. The system 2300 may include an orifice or other flow restriction to control an amount of treatment fluid introduced to the system during negative pressure treatment. In one embodiment the administration of treatment fluids is controlled via the use of an intravenous (IV) fluid giving set such as a Baxter® EMC 9608 Admin Set, B. Braun Medical® Single Chamber IV Infusion Set or similar sterile IV infusion therapy set which is connected to the unit 2002 via the fluid port 2025. The fluid flow rate of treatment fluid being introduced to the supply conduit 2012 is controlled via a roller clamp in the set, which is adjusted to vary the flow restriction within the section of tube that interfaces with the roller clamp component. In this embodiment the rate of fluid instillation can be visually checked via the drip chamber of the IV infusion set when the chamber is orientated upright, with any further flow adjustments made via the roller clamp adjustment. This embodiment provides a manual means to introduce a treatment fluid to the wound 2004 via the wound treatment device 2003.

In an alternative embodiment the vacuum unit 2002 may be connected to an infusion pump via the fluid port 2025 to allow fluids to be supplied to the wound treatment device 2003 in a selectable and controllable manner. Such infusion pump systems could include the B. Braun Medical® Vista® basic large volume infusion pump or the BD® Alaris® Syringe Module for example, which can controllably deliver from 0.1 ml/hour to 1200 ml/hour of treatment fluid on either an intermittent or constant fluid delivery basis. These systems typically offer the means to select the amount, flow rate and frequency of which treatment fluid is dispensed. When treatment fluid is introduced into the vacuum unit 2 the system detects the subsequent drop in the set vacuum pressure level at the Pv and/or Pp pressure sensor(s) and activates the pump assembly 2015 to maintain the systems target level of vacuum pressure. A control algorithm is described in more detail below.

In the embodiments of FIGS. 64 and 65, the vacuum unit 2 comprises a connection manifold 2021 providing a connection interface between the conduit 2005a, 2005b to the treatment device 2003 and the vacuum unit 2002 and between the vacuum unit 2002 and the treatment fluid reservoir 2026 via the fluid port 2025. The connection manifold 21 is illustrated by the dotted line in FIGS. 64 and 65 and replaces connector 2009 shown in FIG. 62. The manifold is described in more detail below.

As described, the treatment system 2100, 2200, 2300 comprises a reservoir 2006 for collecting liquids removed from the wound site 2004, for example, wound exudate. In a preferred embodiment, the reservoir 2006 is positioned at the furthermost position away from the wound and therefore is downstream of the pump assembly 2015, for collecting fluids removed from the wound after they have passed through the pump assembly 2015. In the embodiments shown, the reservoir 2006 comprises a flexible bag. Alternatively, a rigid reservoir could be provided.

The reservoir 2006 comprises one or more air permeable filters or vents 2006a provided in a wall of the reservoir, for example a hydrophobic venting membrane provided over an aperture in the impermeable membrane. The air-permeable filter(s) or vents(s) allow the venting of gases and thereby prevent pressure build-up in the reservoir preventing effective pumping. An example reservoir has eight vents 2006a each having an 8 mm diameter and a pore size of 3 micron to sustain a high level of airflow passing through the system.

Blood clots, fibrin and other solidified fluids or tissue debris may block the venting membranes which causes the bag to inflate with air introduced to the fluid path. This inflation can cause the bag to pop and leak fluid or can prohibit the pump from generating vacuum pressure required by forcing the outlet valves from opening under excess positive pressure.

To avoid these issues a high salt compatible sodium polyacrylate polymer, or other equivalent blood compatible superabsorbent polymers may be added to the reservoir to solidify the blood and wound fluid in the bag. These polymers are available either as loose particles, particles suspended within a dissolvable PVA film pouch or polymer suspended within a textile/fabric like medium. The use of this polymer in tandem with one or more vents on the bag avoids bag inflation and allows the fluid path of the treatment system to cope with much more air as it is introduced into the system.

The pump assembly 2015 includes an inlet and outlet and is driven by a motor. In one embodiment, the pump assembly 2015 may be substantially as described in PCT/NZ2021/050205, comprising a swash plate a plurality of flexible chambers (diaphragms), a plurality of pairs of flexible valves, each pair of valves being in fluid communication with a respective flexible chamber, and a pump inlet and outlet.

The pump assembly 2015 comprises a fluid flow path through the pump from the pump inlet to the pump outlet. In a preferred embodiment the exudate reservoir 2006 is downstream of the pump assembly 2015. This means fluid from the wound passes through the pump assembly 2015.

The pump assembly 2015 preferably comprises a high capacity pump configured to maintain a negative pressure while introducing significant volumes of air to the treatment system 2100, 2200, 2300 with the air inlet valve 2018 open for a significant time portion of a valve open and close cycle time. A large capacity pump assembly 2015 is required to move the increased amount of air and lift fluid from the wound 2004 to the exudate reservoir 2006 while continuing to maintain a negative pressure at the wound 2004 at an effective negative treatment pressure level.

The air inlet valve 2018 may include an actuator such as a solenoid in electrical communication with the controller to drive the valve between open and closed positions. The air inlet valve 2018 does not operate as a pressure relief valve, i.e. the air inlet valve is not controlled to ‘crack open’ to limit a pressure at the wound. The air inlet valve is opened and closed based on a predetermined time period, i.e. the control of the air inlet valve is temporal control, not pressure control, as explained in more detail below.

The fluid inlet valve 2022 may include an actuator such as a solenoid in electrical communication with the controller to drive the valve between open and closed positions.

Dual lumen conduits may be provided for connecting between the vacuum unit 2002 and the treatment device 2003. The conduit may have a circular outer wall. This conduit is preferred for wounds treatments where the conduit must be subsequently removed without opening the wound. The round or circular outer wall allows the conduit to be rotated upon removal to gently release tissue adhered to the side of the conduit which can cause discomfort to the patient.

System Operation

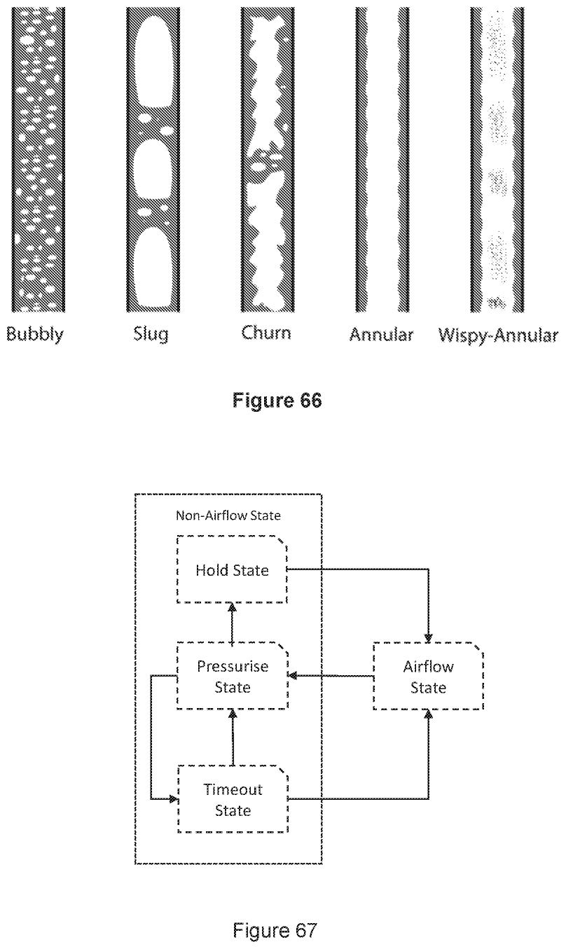

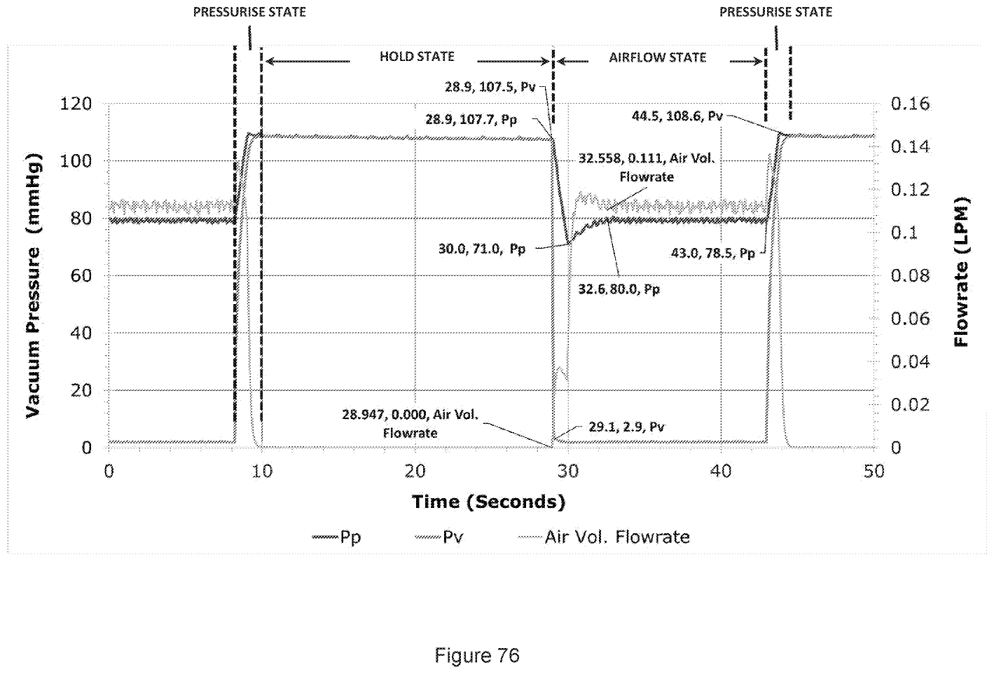

Operation of the treatment system 2100 described above with reference to FIGS. 62 and 63 is now described with reference to FIGS. 66 to 76. The system 2100 comprises the user interface 2014 to allow a user to operate the system. The user interface 2014 may provide visual (e.g. LEDs) and audio indication to the user of system settings and allows inputs, for example one or more buttons, for example to turn the unit on/off, operate the pump or select operation modes. The controller provides system logic and control algorithms in electrical communication with the air valve actuator, pump motor 2013 and pressure sensor(s) Pv, Pp to control the air inlet valve 2018 and the pump assembly 2015. The controller may also communicate with power management and sensor circuits to manage the power supply, for example to provide a battery charge indication to the user via the user interface.

The controller is configured to operate the pump assembly 2015 to maintain a negative pressure at the wound 2004 via the wound treatment device 2003 while opening and closing the air inlet valve. The air inlet valve 2018 is opened to introduce air to the wound site while the pump assembly continues to run to maintain a negative pressure at the wound.

Negative pressure treatment can result in a stagnant system, even when the wound continues to produce exudate. In a stagnant system, the system is effectively sealed from the ambient environment and no fluid transfer or flow is achieved from the wound to the exudate reservoir 2006. This can exacerbate system blockages due to coagulation of blood, fibrin etc at the wound and/or elsewhere in the system. A blockage ultimately results in failure to provide negative pressure at the wound, defeating the negative pressure treatment.

In order to avoid a stagnated system, the controller opens and closes the air inlet valve 2018 while continuing to run the pump assembly 2015 to maintain a negative pressure at the wound.

For example, the treatment system 2100 is configured to open the air inlet valve 2018 to introduce air to the wound site while maintaining a vacuum pressure (a first vacuum pressure) at the wound site 2004 wound treatment device 2003 of at least 40 mmHg, and preferably at least 50 mmHg. In an example embodiment the treatment system is capable of maintaining vacuum pressure at the wound site/wound treatment device of approximately 50 mmHg to 100 mmHg, or approximately, 60 mmHg to 100 mmHg, or 70 mmHg to 100 mmHg, or 80 mmHg to 100 mmHg, with the air inlet valve open introducing air to the wound. When the controller closes the air inlet valve, the pump continues to operate to maintain negative pressure at the wound. With the air valve closed the vacuum pressure at the wound site 2004 may be around 100 mmHg to 150 mmHg (a second vacuum pressure).

Preferably the vacuum pressure maintained at the wound treatment device when the air inlet valve is open is at least a substantial portion of the vacuum pressure maintained at the wound when the air inlet valve is closed, or may be equal to the vacuum pressure maintained at the wound when the air inlet valve is closed. For example, the vacuum pressure maintained at the wound with the air valve open may be approximately 30% to 100% of the vacuum pressure maintained at the wound with the air valve closed, or approximately 50% to 100%, or 70% to 100%, or about 80% of the vacuum pressure maintained at the wound with the air valve closed.

With the air inlet valve closed, the vacuum pressure at the wound may be about 20 to 50 mmHg higher than the vacuum pressure at the wound when the air inlet valve is open, or may be equal to the vacuum pressure at the wound when the air inlet valve is open.

In a preferred embodiment the system is configured to cycle the air inlet valve between the open and closed positions while continuing to maintain a negative pressure at the wound. When the air inlet valve is closed the system reverts quickly to a stagnant state. To avoid remaining in a stagnant state that could lead to blockages forming, the controller is configured to again open the air inlet valve while maintaining a negative pressure at the wound, and then again close the air inlet valve. The opening and closing of the air valve continues. The introduction air of into the system while maintaining a negative pressure at the wound promotes movement of fluid from the wound to the reservoir and reduces the risk of blockages. In some embodiments, the treatment system may be configured to continue to open and close the air inlet valve to achieve continuous operation of the pump to maintain fluid flow and avoid remaining in a no-flow or stagnant state for an extended period.

In a preferred embodiment the system is configured so that with the air inlet valve 2018 open, the system achieves an equilibrium state, with a flow rate of air into the treatment system through the air inlet valve 2018 equal to a flow rate of fluid (e.g. exudate) and air through the pump. In an equilibrium state, the vacuum pressure at the wound treatment device 2003 is maintained at or reaches a steady state or constant vacuum pressure level (the first vacuum pressure). The system may achieve the constant vacuum pressure level after a short duration, for example several seconds or less, for example 5 second or less. In some embodiments, with the air valve open and in an equilibrium state, the pressure drop across the treatment device is substantially zero, with substantially all of the pressure drop between the system vacuum pressure and ambient pressure occurring across the inlet restriction, provided for example by the air inlet filter. In some embodiments, with the air inlet valve open and in an equilibrium state, the pressure drop across the treatment device is constant. Introducing air to the wound can create a pressure drop across the wound site—between an upstream side of the treatment device and a downstream side of the treatment device—allowing for the transfer of fluid from the wound 2004 to the reservoir 2006, to thereby reduce the risk of coagulation and system blockage.

With the air valve closed, the pump is controlled to maintain a negative pressure at the wound and a flow rate from the wound to the pump is proportional to the patient's wound response; i.e. the flow rate is proportional to the exudate produced at the wound. With the air inlet valve closed, the pump is controlled to maintain the vacuum pressure at the wound treatment device at a steady state or constant vacuum pressure level (the second vacuum pressure). Again, the system may achieve the constant vacuum pressure level after a very short duration, for example several seconds or less, for example 5 second or less. As described above, the first vacuum pressure is less than or equal to the second vacuum pressure.

The steady state vacuum pressure at the wound treatment device 2003 with the air inlet valve 2018 open may be less than the steady state vacuum pressure at the wound treatment device with the air inlet valve closed. However, the vacuum pressure at the wound treatment device 2003 with the air inlet valve open is sufficient for effective negative pressure treatment. As described above, the first vacuum pressure is at least a substantial portion of the second vacuum pressure and may be equal to the second vacuum pressure. Thus, the cyclic opening and closing of the air inlet valve while running the pump to continuously achieve a negative treatment pressure not only improves removal of exudate and reduces the risk of system blockages, but also maintains the negative pressure environment at the wound for effective wound treatment.

Cycling the air inlet valve open and closed while maintaining a negative pressure at the wound achieves a reduced fluid density at the wound site by the introduction of air. Often a height differential exists at the wound site, for example when the patient is upright or in a standing position. A height differential at the wound can result in fluid remaining static in a lowermost location in the wound, with flow in only upper portions of the wound. By introducing air across the wound site, air reaching the lowermost portions of the wound can lift fluid from those lowermost portions and improve fluid movement throughout the wound. The introduction of air essentially allows the system to operate much like an air pump to allow lower density fluid to move ‘uphill’ or against gravity.

The inventors have additionally identified a preferred mode of operation whereby the air valve is operated between open and closed positions while maintaining a negative pressure at the wound in order to introduce a flow rate of air into the system that achieves a ‘bubbly flow’ or a ‘slug flow’ from the wound site to the reservoir. FIG. 66 illustrates a range of flow types in a fluid comprising both liquid and gas states. Introducing too much air due to leaving the air inlet valve open for too long can result in an annular type flow with exudate flowing along the inner wall of the conduit and air flowing through the middle of the conduit.

This can cause the exudate to become stagnant on the wall of the conduit which can lead to the fluid solidifying. A layer of solidified fluid can increase over time leading to a blockage. By cycling the air inlet valve open and closed, liquid exudate can reform a uniform column within the flow path of the system when the air valve is closed, with subsequent opening of the air inlet valve to introduce air results in bubbles or slugs of air passing through the exudate. The air valve is again closed before an annular type flow is achieved. The inventors believe that this results in an improved removal of exudate and reduction in blockages.

An example implementation of cycling the air inlet valve between open and closed during NPT is now described with reference to FIGS. 67 to 71. As illustrated in FIG. 67, the controller is configured to implement an airflow mode or state in which the air inlet valve is opened and the pump is operated to achieve a negative pressure at the wound, and a non-airflow mode or state in which the air inlet valve is closed and the pump is operated to achieve a negative pressure at the wound. In the illustrated embodiment the non-airflow state comprises a pressurise state, a hold state and a timeout state.

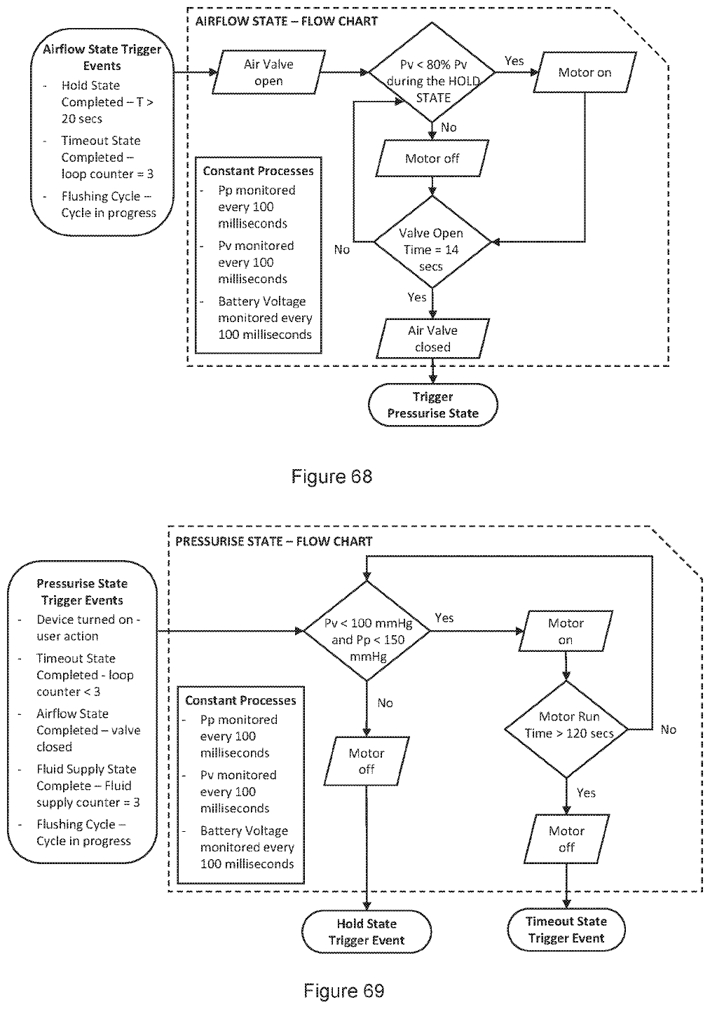

With reference to FIG. 68, in the airflow state the controller opens the air inlet valve to allow air to enter the system on the upstream side of the treatment device and runs the pump to achieve a pressure threshold. For example, if the pressure sensed by the pressure sensor Pp at the downstream side of the treatment device is less than a pressure threshold, the controller runs the pump (turns the pump on). In other words, if the pressure at Pp is greater than or equal to the threshold pressure, the controller turns the pump off.

In the illustrated embodiment, the pressure threshold at the downstream side of the treatment device (Pp) is a portion of a pressure threshold at the upstream side of the treatment device (Pv) when the air inlet valve is closed. In the illustrated embodiment, the pressure threshold at the downstream side of the treatment device (Pp) is 80% of a pressure threshold at the upstream side of the treatment device (Pv) when the air inlet valve is closed. For example, when the air inlet valve is closed, the pressure threshold at the upstream side of the treatment device at Pv is 100 mmHg, and in the airflow state with the air inlet valve open, the pressure threshold at Pp is 80 mmHg.

The pump may repeatedly turn on and off, e.g. under PID control by the controller, to maintain the vacuum pressure at the downstream side of the wound treatment device with the air inlet valve open. Preferably the system is configured to achieve the threshold pressure at the downstream side of the treatment device at Pp in a very short time period, i.e. within several seconds or less, for example 5 second or less. The air inlet valve remains in the open position for a time period. When the air inlet valve is open, the pressure at the wound is maintained constant. In the illustrated embodiment, the air inlet valve remains in the open position for 14 seconds. Once 14 seconds has elapsed, the controller closes the air inlet valve and the controller moves to the pressurise state of the non-airflow state.

The parameters of the above described airflow state are provided by way of example. In some embodiments, the system may be without the pressure sensor Pp on the downstream side of the treatment device. The pump may be provided with a suitable capacity such that the pump is run at a predetermined rate corresponding with a particular system performance to achieve a known or acceptable pressure level at the wound treatment device (the first vacuum pressure) with the air inlet valve open. Additionally, or alternatively, the system may include a pressure relief valve to introduce air to the system at the pump inlet to ensure the vacuum pressure generated by the pump does not increase too high. However, in the preferred embodiment the system includes pressures sensor Pp and the controller operates the pump so that the pressure does not increase beyond a predetermined pressure threshold, being 80 mmHg in the above example. Other pressure thresholds are possible depending on a desired treatment regime. Preferably the controller implements PID control to achieve accurate control of the pump and therefore control of the vacuum pressure at the wound. The controller may use a pulse-width modulation (PWM), or pulse-duration modulation, method in the control of the pump motor.

As shown in FIGS. 62 to 65, in the example embodiments the pressure sensor Pv is on an ambient side of the filter. The sterile filter 2019 presents a known pressure drop to prevent the vacuum pressure at the treatment device collapsing to ambient pressure when the air inlet valve is open. With the pressure sensor Pv on the ambient side of the filter the sensor Pv essentially measures ambient pressure when the air inlet valve is open. Thus, when the air inlet valve is open, the pressure sensed by sensor Pv is not used in the control of the pump, the pump will run until the pressure sensed by Pp increases above the pressure threshold. In some embodiments, the pressure at Pp will not reach the pressure threshold when the valve is open. The pump may run continuously when the air inlet valve is open, however this is less preferred.

With reference to FIG. 69, in a pressurise state, the air inlet valve is closed, and the controller runs the pump to achieve a pressure threshold to achieve a known or acceptable vacuum pressure at the wound treatment device (the second vacuum pressure). With the air valve closed the vacuum pressure at the wound treatment device may be increased compared to the vacuum pressure achieved in the airflow mode. In the illustrated embodiment, if the pressure sensed by the pressure sensor Pv at the upstream side of the treatment device is less than a 100 mmHg, and the pressure sensed by the pressure sensor Pp at the downstream side of the treatment device is less than 150 mmHg, the controller runs the pump. In other word, if the pressure Pv is greater than or equal to 100 mmHg or pressure Pp is greater than or equal to 150 mmHg, the controller turns the pump off.

The system may be configured to achieve the threshold pressure after a very short duration of closing or opening the air inlet valve, i.e. within several seconds or less, for example 5 second or less. With the air valve closed, since the system is closed or sealed, the system reaches a stagnant or no flow condition very quickly with zero pressure drop across the treatment device and therefore with the pressure at Pv=the pressure at Pp. In the illustrated embodiment, since the pressure threshold at Pv is less than the pressure threshold at Pp, the controller controls the pump based on the upstream pressure sensor Pv, the lower of the two pressure thresholds. However, a pressure drop through the system may occur when tissue debris and/or solidifying materials such as fibrin accumulate within the would treatment device and/or the pump, in which case a pressure differential may develop between the upstream and downstream sides of the treatment device as measured by sensors Pv and Pp. System restrictions may cause the system pressure to reach the higher threshold at the downstream side of the treatment device, before the lower threshold is reached at the upstream side of the treatment device, in which case the pump is controlled based on the downstream pressure sensor Pp to the higher pressure threshold at Pp.

Once the pressure threshold has been reached the controller turns the pump off and moves into a hold state. The pressurise state includes a time-out check so that if the pump has not achieved the pressure threshold (e.g. at Pp) within 120 seconds the motor is turned off and the controller moves to a time out state. This may occur for example due to a blockage within the system or other failure mode, such as a leak.

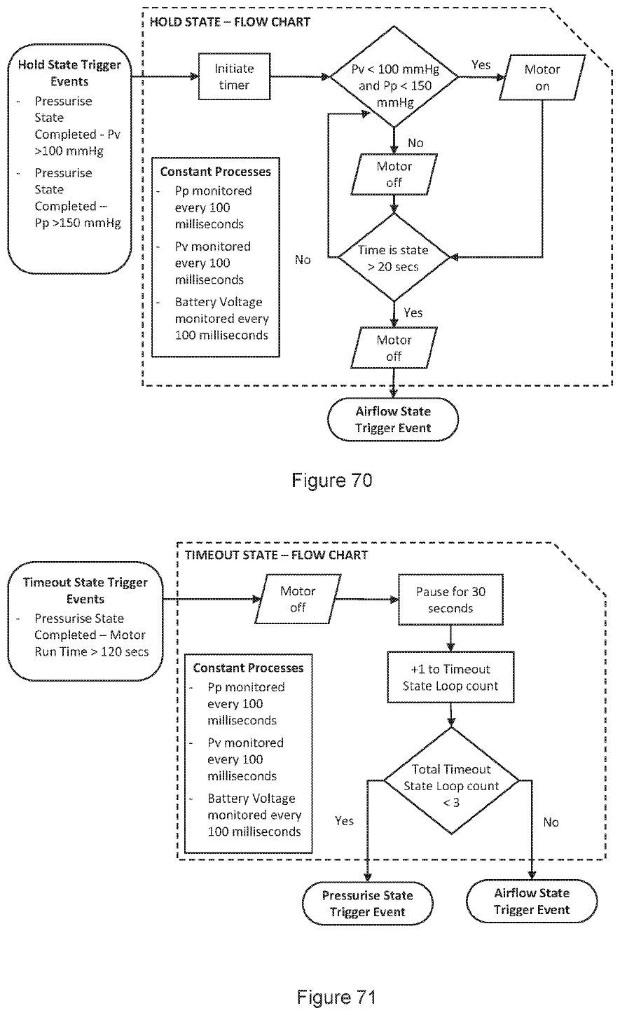

With reference to FIG. 70, in a hold state the controller maintains the air inlet valve in the closed position and continues to operate the pump to maintain the desired or acceptable vacuum pressure at the wound treatment device, by turning the pump on and off, for example under PID control to achieve a desired pressure threshold at Pv or Pp. The controller maintains the vacuum pressure with the air inlet valve shut for a time period. In the illustrated embodiment, the air inlet valve is closed for 20 seconds. Once 20 seconds has elapsed, the controller returns to the air flow mode and the cycling of the opening and closing of the air inlet valve is repeated. The opening and closing of the air inlet valve may be cycled continuously to achieve the above described benefits.

The above example implementation provides an air inlet valve open time of 14 second and an air inlet valve close time of 20 seconds. These time periods are by way of example and alternative time periods may be implemented. However, it is to be noted that the air inlet valve is open for a substantial portion of a total open/close cycle. In this embodiment, the total open/close cycle, or the ‘cycle pitch’ is 34 seconds, with the air inlet valve open for 14 second of the 34 second period, or around 40% of the total cycle. In some embodiments, the air inlet valve is open for at least 10% of the cycle pitch, or at least 20% of the cycle pitch, or at least 30% of the cycle pitch, or at least 40% of the cycle pitch. For example, the air inlet valve open time period may be around the same as the close time period (50% of the cycle pitch). In some embodiments, the air inlet valve may be open for more than 50% of the total cycle.

The above example system configuration provides a cycle time of 34 seconds. However longer or shorter cycle times are possible. As described above, the opening and closing of the air inlet valve required to achieve a slug or bubbly flow from the would site to the reservoir while maintaining negative pressure at the wound is ideal. A maximum valve cycle time may be 1 minute or several minutes. However, the air inlet valve should be open for at least approximately 10 seconds at the above pressures to ensure sufficient air is introduced to the system. The air inlet valve may be open for 10 to 40 second in each air inlet valve open/close cycle.

The time periods for which the air inlet valve is open and closed is dependent on the air inlet flow restriction, the pump capacity, the treatment device configuration and the supply and exudate conduit length and diameter. The above described system components and control parameters are provided by way of example. However, the inventors believe that the system parameters should be selected to enable the air inlet valve to be open for a significant duration while maintaining the negative pressure at the wound at a level useful in the negative pressure treatment of a wound.

With reference to FIG. 71, the example embodiment includes a time out state to safely manage a situation whereby the system is unable to reach an intended negative pressure level. As described above with reference to FIG. 69, if the system is unable to pressurise when the air inlet valve is closed after a predetermined time period (for example 2 minutes) the controller enters the time out state. The controller pauses the pump operation for 30 second and increments a timeout counter. If the time out counter is less than a predetermined count threshold, the controller then returns to the pressurise state to try and pressurise the wound treatment site. If the timeout counter threshold is reached, the controller returns to the air flow state. As described above, introducing air can reduce blockages. The system may have failed to pressurise due to a blockage. Returning to the air flow state may remove a blockage before returning to the pressurise state.

In some embodiments, the treatment system may implement other control parameters not presented in FIGS. 67 to 71. For example, in some embodiments, the system comprises the pressure sensor Pv on the upstream side of the treatment device and the pressure sensor Pp on the downstream side of the treatment device. The controller may operate the pump and/or air inlet valve based on a pressure differential measured between the two pressure sensors. For example, the controller may open the air inlet valve when the pressure differential increases above an upper threshold or is above an upper threshold for a predetermined time period. A system pressure differential may be indicative of a blockage in the system, especially when the air inlet valve is closed. With the air valve closed and with the system in a stagnant state, the pressure on the upstream and downstream sides of the treatment device should be substantially equal. The controller may close the air valve when the pressure differential decreases below a lower threshold or is below a lower threshold for a predetermined time period. The controller may stop the pump and/or the airflow state when the pressure differential increases above an upper or maximum threshold.

As described above with reference to FIGS. 64 and 65, in some embodiments the system is configured to introduce a treatment fluid to the wound. For the system of FIG. 64, the controller may be configured to operate the treatment fluid inlet control valve 2022 to introduce treatment fluid in a similar way to operation of the air inlet valve 2018. The treatment fluid reservoir 2026 is preferably at ambient pressure.

The controller opens the fluid inlet valve 2022 while operating the pump to maintain a negative pressure at the wound treatment device, to draw treatment fluid into the treatment device. In a preferred embodiment, the system is configured so that with the fluid inlet valve 2022 open, the system achieves an equilibrium state, with a flow rate of treatment fluid into the treatment system from the treatment fluid reservoir 2026 is equal to a flow rate of fluid (e.g. exudate and treatment fluid) through the pump. In an equilibrium state, the vacuum pressure at the wound treatment device is maintained at or reaches a steady state or constant vacuum pressure level (i.e. a third vacuum pressure). The system may achieve the constant vacuum pressure level after a very short duration, for example several seconds or less, for example 5 second or less. In a preferred embodiment, with the fluid inlet valve open and in an equilibrium state, the pressure across the treatment device is substantially zero.

When the fluid inlet valve is open, the controller may operate the pump to achieve the same pressure at the treatment device that the treatment system achieves when the air inlet valve is open.

With the fluid inlet valve closed, the pump is controlled to maintain a negative pressure at the wound. With the fluid inlet valve closed, the pump may be controlled to maintain the vacuum pressure at the wound treatment device at a steady state or constant vacuum pressure level (a fourth vacuum pressure). Again, the system may achieve the constant vacuum pressure level after a very short duration, for example several seconds or less, for example 5 second or less. When the fluid inlet valve is closed, the controller may operate the pump to achieve the same pressure at the treatment device that the treatment system achieves when the air inlet valve is closed.

The steady state vacuum pressure at the wound treatment device with the fluid inlet valve open may be less than the steady state vacuum pressure at the wound treatment device with the fluid inlet valve closed. However, the vacuum pressure at the wound treatment device with the fluid inlet valve open is sufficient for effective negative pressure treatment. The treatment fluid is not introduced under a positive pressure. Thus, the opening and closing of the fluid inlet valve while running the pump to continuously achieve a negative treatment pressure not only maintains the negative pressure environment at the wound for effective treatment but also provides for the installation of treatment fluid to improve treatment, the removal of exudate, and reduce the risk of system blockages.

The amount of treatment fluid administered to the system can be controlled based on the time the fluid inlet valve is open. A flow restriction (such as a constricting orifice) may be placed between the treatment fluid reservoir 2026 and the Pv pressure sensor positioned upstream of the wound treatment device. The resultant pressure drop across this restriction can allow the rate of fluid to be determined from the resulting pressure drop measured by the sensor Pv and the total amount of treatment fluid administered to be calculated. Alternatively, the treatment fluid inlet valve may be open until a differential pressure threshold is achieved or achieved for a time period, or the valve may be opened for a predetermined time period. The treatment fluid inlet valve is preferably opened when the air inlet valve is closed.

With reference to the embodiment of FIG. 65, the system is without a treatment fluid inlet valve controlled by the controller. The system administers treatment fluid during negative treatment pressure since the vacuum pressure at the wound draws the ambient treatment fluid into the system. When the air valve is open, air flows to the treatment device, and the flow of air into the system tends to stop the flow of fluid from the treatment fluid reservoir due to the much lower density of the air compared to the density of the treatment fluid. When the air inlet valve is closed, the negative pressure at the wound draws fluid from the treatment fluid reservoir into the system and flood into the wound. Treatment fluid passes through the treatment device and wound and through the pump to the reservoir as the pump maintains a vacuum pressure at the wound. Reopening the air valve again stops the flow of treatment fluid and causes a pressure differential to move fluid comprising treatment fluid and exudate from the wound. Thus, cycling the air inlet valve can also achieve addition and removal of treatment fluid to and from the wound in a cyclic manner. The amount of treatment fluid added is dependent on how long or how much air has been introduced. The amount of treatment fluid introduced to the system may be proportional to an amount of air introduced to the system.

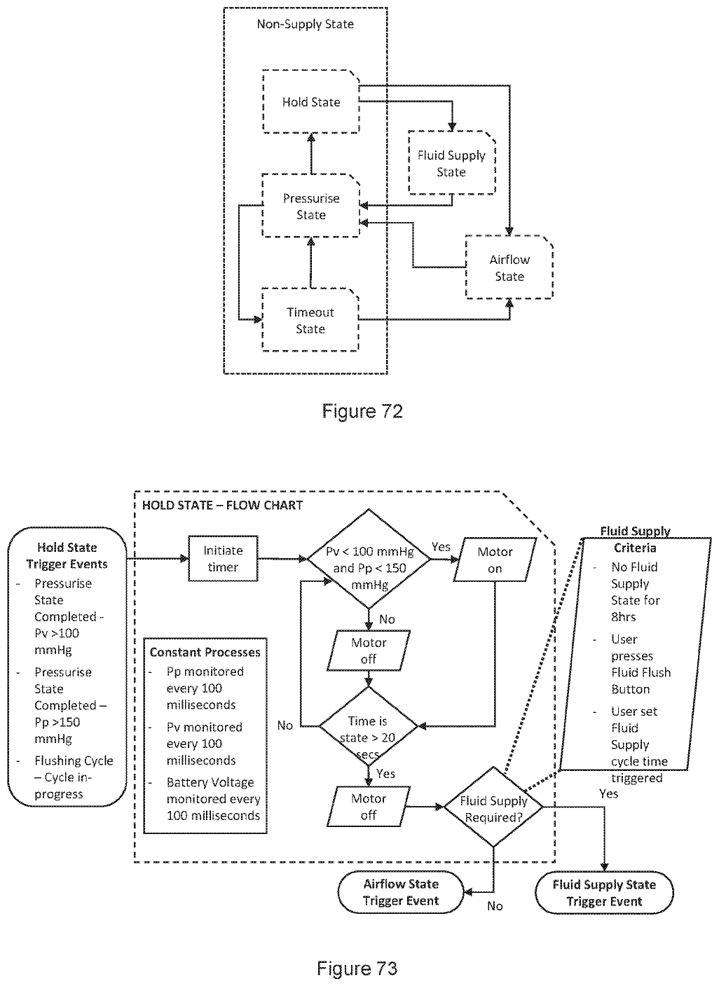

An example implementation of the system of FIG. 64 is now described with reference to FIGS. 72 to 75. As illustrated in FIG. 72, the controller is configured to implement a fluid supply mode or state in addition to the airflow state described above. The controller implements a non-supply/non-airflow mode in which the air inlet valve and the treatment fluid valve are closed and the pump is operated to achieve a negative pressure at the wound. In the illustrated embodiment the non-airflow state comprises a pressurise state, a hold state and a timeout state.

The air flow state and pressurise state of FIG. 72 are as described above with reference to FIGS. 68 and 69. Once the airflow state and pressurise state of FIGS. 68 and 69 have been run the controller implements the fluid supply hold state of FIG. 73.

With reference to FIG. 73, in the hold state the controller maintains the air inlet valve in the closed position and continues to operate the pump to maintain the desired or acceptable vacuum pressure at the wound treatment device, by turning the pump on and off, for example under PID control to achieve a desired pressure threshold (at Pp and/or Pv). The controller maintains the vacuum pressure with the air inlet valve shut for a time period, e.g. 20 seconds. Once 20 seconds has elapsed, the controller turns the pump off and checks to see if the fluid supply state is required. If the fluid supply state is not required, the controller returns to the air flow mode and the cycling of the opening and closing of the air inlet valve is repeated as described above with reference to FIG. 67. The controller implements the fluid supply state if no treatment fluid supply has been provided for a predetermined time period, for example 8 hours, or a user set fluid supply cycle time is triggered, or if a user manually requests a fluid supply, for example by pressing a button on the user interface of the vacuum unit.

The time period between activating the fluid supply state is much greater than the air inlet valve open and close cycle time period. For example, the air inlet valve cycle time period may be less than 1 minute and the time period between fluid supply states may be more than 1 hour

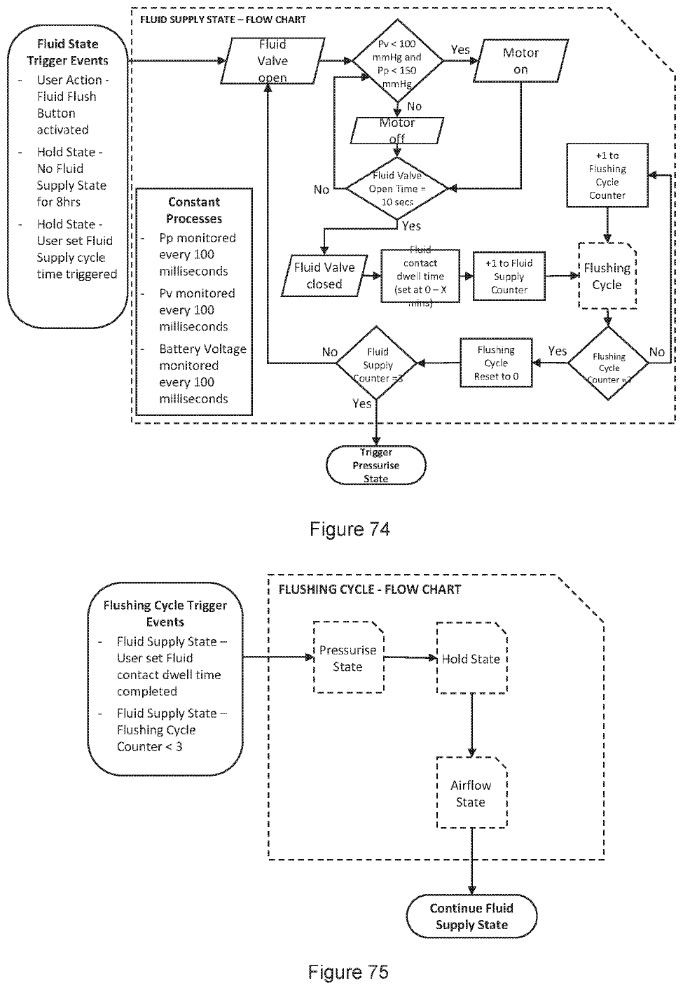

With reference to FIG. 74, in the fluid supply state the controller opens the fluid valve to allow the treatment fluid to flow from the treatment fluid reservoir to the upstream side of the treatment device and runs the pump to achieve a pressure threshold. If the pressure sensed by the pressure sensor Pv at the upstream side of the treatment device is less than 100 mmHg, and the pressure sensed by the pressure sensor Pp at the downstream side of the treatment device is less than 150 mmHg, the controller runs the pump. The control of the pump when the treatment fluid valve is open may be the same or similar to the pump control when the air inlet valve is open as described above. In the illustrated example the controller maintains the fluid valve open for 10 seconds, however other time periods are possible. The controller closes the fluid valve and may allow for a fluid contact dwell time to allow the fluid introduced to the wound to flood or remain in the wound site for a set period of time. The controller may allow for a user input to set the dwell time of between 0 minutes to 10 minutes or other time period. Following the delay to allow fluid contact within the wound the controller enters a flushing cycle to flush the treatment fluid from the wound. In the illustrated embodiment the controller repeats the flushing cycle three times, however the controller may perform the flushing cycle once, twice or more than three times. In the illustrated embodiment the controller repeats the fluid supply state three times before returning to the pressurise state, however the controller may perform the fluid supply state once, twice or more than three times.

With reference to FIG. 75, in the flushing cycle the controller steps through the pressurise state, hold state and airflow state as described above with reference to FIGS. 69, 73 and 68 respectively, before continuing with the fluid supply state to repeat the fluid supply state to open the fluid valve again if required as shown in FIG. 74. At the conclusion of the fluid supply state the controller returns to the pressurise state of FIG. 69. The system continues to pressurise, hold pressure and cycle the air inlet valve open and closed as described above.

In the illustrated embodiment, the fluid inlet valve is open for 10 seconds and closed for 102 seconds in each open and close cycle of the fluid inlet valve. The close time is dependent on the dwell time and the combined flushing cycle run time. In the illustrated embodiment, the fluid supply state includes three flushing cycles. With each flushing cycle requiring 34 seconds, and for an example dwell time of zero, in the illustrated example the fluid supply valve is closed for a total of 102 seconds. In the illustrated example the fluid inlet valve is open for around 10% of the cycle pitch. The fluid inlet valve may be open for at least 5% of the cycle pitch, or at least 10% of the cycle pitch, or at least 20% of the cycle pitch.

The fluid supply and flushing states provides a treatment fluid to the wound while maintaining a negative pressure and flushes the treatment fluid from the wound using the introduction of air to remove the fluid and exudate from the wound. As described above, a number of treatment fluid flushes may be provided. This procedure reduces stagnated fluid in the wound, thereby reducing blockages in the system and ensure negative pressure to be continually applied to the wound site.

The operation of the system 2100, 2200, 2300 may be via the user interface 14, which enables a user to selectively operate the system. The user interface may provide visual (e.g. LEDs) and/or audio indication to the user to communicate system settings. The user interface 2014 may includes several buttons to initiate or cease the delivery of negative pressure to the connected wound treatment device 2003, turn the unit power on or off, silence the audible alarm and/or connect the device to a remote wireless receiving device to transmit data regarding the operation or status of the system.

The controller may provide system logic and control algorithms in electrical communication with the actuator for the air valve 2018, the motor of the pump 2015, and pressure sensors Pv, Pp. The controller 2017 is configured to control the air inlet valve 2018, and the pump assembly 2015 based on the readings at the pressure sensors Pv, Pp. The controller may also communicate with power management and sensor circuits to manage the power supply or provide battery level warning alarm.

The controller 2017 is configured to operate the pump assembly 2015 to maintain a negative pressure at the internal wound 2004 via the implanted wound treatment device 2003 while opening and closing the air inlet valve 2018. The air inlet valve 2018 is opened to introduce air to the wound site while the pump assembly continues to run to maintain a negative pressure at the wound as described elsewhere within this specification.

Negative pressure treatment can result in a stagnant system that can exacerbate system blockages due to coagulation of blood, fibrin etc at the wound and/or elsewhere in the system. A blockage can ultimately result in failure to provide negative pressure at the wound, reducing the effectiveness of the negative pressure treatment.

The controller may be configured to adapt to anticipated changes that can occur system in response to the changes occurring at the wound treatment site 2004 and implanted treatment device 2003. As the treatment device is subjected to repeated cycles through the pressurise, hold and airflow state it has been discovered that a pressure differential between the Pv and Pp pressure sensors can occur in response to changes in the treatment site 4 and/or implanted wound treatment device 2003 as a result of tissue in-growth, accumulation of wound debris and many other factors.

In response to these dynamic changes the system adjusts the target pressure level being applied at the Pv pressure sensor during the pressure site to compensate for the changes in the treatment device 2003. For example, if the motor has stopped as a result of the Pp pressure sensor being above 150 mmHg the system may be configured drop the target vacuum pressure level from for example, the Target 1 (100 mmHg) pressure being applied at the Pv pressure sensor by 10 mmHg to a Target 2 pressure of 90 mmHg before advancing to the hold state. If the pressure drop across the implanted treatment device 2003 increases again the system will continue to drop the target level by one step until the Pv pressure level reaches a pressure below 60 mmHg (Target 5).

| Pv Target Level | Pressure Level |
|---|---|
| Target 1 | Pv = 100 mmHg |
| Target 2 | Pv = 90 mmHg |
| Target 3 | Pv = 80 mmHg |
| Target 4 | Pv = 70 mmHg |
| Target 5 | Valve closed |

Once the pressure level measured at the Pv pressure sensor reaches this level the system will then halt the transition from the hold state to the airflow state which will revert the system to a continuous vacuum pressure level system.

If the vacuum pressure level at Pv returns to 90 mmHg (Target 2), following a drop to below 60 mmHg (Target 5) during the hold state, the system will resume the advancement to the airflow state where the cycling between hold, airflow and pressurise will resume.

### Animal Studies

A series of animal studies were performed to compare the effect of various valve cycle timings on clinical outcomes for seroma prevention within a unilateral ovine external abdominal oblique dead space seroma model.

The animal studies utilised an implanted wound treatment device 2003 similar in shape to the device 1201 illustrated in FIG. 46. The implanted wound treatment device 2003 comprised a perforated central conduit approximately 260 mm long comprising a repeating row of four Ø0.5 mm+0.2 mm sized perforations spaced approximately 2 mm apart along a central conduit with an approximate internal conduit area of 18 mm² (equivalent internal tube diameter of Ø4.8 mm). The implanted wound treatment device had an outer diameter of approximately 120 mm with an inner diameter of approximately 60 mm.

A removal conduit 2011 approximately 1000 mm long with an internal diameter of 3/16″ (Ø4.8 mm ID) was connected to a downstream end of the perforated central conduit, with a supply conduit 2012 approximately 1000 mm long with an internal diameter of 1/16″ (Ø1.6 mm ID) was connected to an upstream end of the central conduit.

Each implanted wound treatment device was connected to an externally mounted vacuum device 2 constructed to reflect the embodiment treatment system 2100 represented in FIG. 63, with the treatment algorithm as described above in relation to FIGS. 67 to 71.

The external vacuum pump device 2002 connected to this implant 2003 was configured to open the air inlet valve for 14 Seconds, with the closed duration time varied to assess the difference in clinical outcomes associated with varying hold lengths. Tests were carried out with a 20 second, 120 second, 240 second and 360 second valve close timing in the ‘HOLD STATE’.

The system was maintained at a vacuum pressure level of 80 mmHg during the instillation of filtered air during the AIRFLOW STATE with the system returning to a second equilibrium pressure of 100 mmHg during the PRESSURISE STATE. This cycle operated in a continuous pattern with the vacuum pressure level along the fluid removal conduit 2011 capped at 150 mmHg as a safety mechanism.

Tests were carried out in five sheep, with each animal receiving a single implanted wound treatment device 2003. A defect site of ˜110 cm² in area was created by excising approximately 60 grams of external abdominal oblique muscle from an undermined area above the muscle. The implant device was positioned at the lowermost ventral aspect of the defect site and was secured to the treatment site using a series of passed sutures that were tied off to affix the implant in place. The removal conduit 2011 and supply conduit 2012 both exiting the wound at the upper and forward most ventral-cranial aspect of the wound with the conduits held in place at the skin portal using stay sutures. Once the treatment site was closed the implant device was connected to the externally mounted vacuum pump device 2002 to function as programmed.

An ultrasound assessment was performed at days 7 post-surgery to assess the size of any seroma forming at the defect site, where the volume of any seroma measured at the defect site was calculated using the formula to determine the volume of an ellipsoid.

The volume of wound exudate collected within the reservoir of the device was measured daily to determine the total amount of fluid collected over the 7 days post-surgery. All animals were euthanised at 14 days post-surgery to perform a gross assessment of the treatment site; with exception of Animal ID 5 which was euthanised 7 days post-surgery. The results from the animal study are shown in the table below.

TABLE 1
Animal study results

| | | | | Ultrasound | Total | |
|---|---|---|---|---|---|---|
| | | | | Seroma | Exudate | |
| | Valve | | Resected | Volume at 7 | Collected | Seroma |
| | Closed | Animal | Muscle | Days Post- | Following | Observed |
| Animal | Time | Weight | Weight | Surgery | 7 Days | Following |
| ID | (seconds) | (KG) | (grams) | (mL) | (mL) | Euthanasia |
| 1 | 20 | 62 | 71.0 | 0 | 369.9 | No |
| 2 | 20 | 60 | 61.8 | 0 | 280.3 | No |
| 3 | 120 | 61 | 63.5 | 0 | 188.9 | No |
| 4 | 360 | 63 | 58.9 | 156.2 | 736.2 | Yes, Large |
| 5 | 240 | 59 | 57.8 | 4.6 | 306.2 | *Yes, Minor |

*Euthanasia performed at 7-days post-surgery.

There were no signs of any seroma or wound fluid at the defect site for animal IDs 1, 2 and 3 following euthanasia at 14 days post-surgery time point, with the implanted wound treatment device 3 found to be completely integrated with the surrounding tissue.

There were moderate signs of a seroma at the defect site for Animal ID 5 which was euthanised 7 days post-surgery, with the result also consistent with the ultrasound assessment at the same time point.

The defect site of Animal ID 4 was found to have a large seroma at the 14 day post-surgery time point which was consistent with the ultrasound findings at the 7-day post-surgical timepoint with virtually zero signs of any integration of the separated tissue planes of the defect site.

The results from this animal study support the conclusion that an air inlet valve closed time of 120 seconds or less is more likely to lead to complete dead space closure and the prevention of seroma formation at the defect site with an animal.

A system as described herein may provide significant benefits, including but not limited to one or more of the following:
Improved fluid removal from the wound site, providing improved healing benefits such as reduced edema by the removal of excess exudate;
Reduced risk of blockages forming in the system;
Maintaining effective negative pressure at the wound even during addition of air to ensure effective treatment;
Removal of exudate from a lower portion of a wound where there is a height differential at the wound;
Low power consumption suited for application in portable wound treatment systems;
Application of treatment fluids to the wound while maintaining effective negative pressure at the wound to ensure effective treatment;
Provision of negative pressure to a larger portion of a treatment space to improve treatment throughout the entire treatment space;
System configurability with and without the provision of a treatment fluid supply to the wound;
Ease of providing a sterile interface between an air inlet and a wound site.

1. A device for implantation at a treatment site in the body of a patient for the removal of fluid from the treatment site; the device comprising a conduit structure at least in part defining a fluid removal lumen, and a porous bioresorbable sheath surrounding a portion of the conduit structure;
wherein the conduit structure comprises a removable component configured for removal from the treatment site upon completion of treatment.

2. A device as claimed in claim 1, wherein the device is configured to deliver a fluid to the treatment site, and wherein the conduit structure further defines a fluid supply lumen.

3. A device as claimed in claim 1 or 2, wherein one end of the fluid supply lumen is in fluid communication with one end of the fluid removal lumen.

4. A device as claimed in any preceding claim, comprising a dual lumen port for connection with one or more external components, wherein a first lumen of the port is in fluid communication with the fluid removal lumen.

5. A device as claimed in any preceding claim, wherein the bioresorbable sheath comprises a plurality of apertures positioned to enable fluid communication between the treatment site and the conduit structure, the apertures each having an area of about 1 mm² or less.

6. A device as claimed in claim 5, wherein the apertures in the sheath each have an area of between about 0.2 mm² to about 0.8 mm².

7. A device as claimed in any preceding claim, wherein the sheath comprises a top sheet that wraps over a top part of the conduit structure, and a bottom sheet that wraps over a bottom part of the conduit structure, wherein the top and bottom sheets are joined around the conduit structure along a side seam.

8. A device as claimed in claim 7, wherein the top and bottom sheets are stitched together.

9. A device as claimed in claim 7 or 8, wherein the sheath forms one or more flange(s) or tab(s) extending beyond the side seam, for securing the device to tissue at the treatment site.

10. A device as claimed in claim 9, wherein the flanges or tabs comprise two layers, and the layers are attached at or near an edge of the flange or tab.

11. A device as claimed in any preceding claim, wherein the apertures in the sheath are provided on upper and lower surfaces of the device.

12. A device as claimed in any preceding claim, wherein the sheath comprises an end section proximal an inlet and outlet of the device, configured to prevent or minimise the ingress of wound debris into the conduit structure.

13. A device as claimed in claim 12, wherein the end section of the sheath does not comprise through apertures.

14. A device as claimed in any preceding claim, an end of the sheath distal an inlet and outlet of the device is closed.

15. A device as claimed in any one of claims 1 to 13, wherein an end of the sheath distal an inlet and outlet of the device is open.

16. A device as claimed in any preceding claim, wherein the sheath comprises one or more layers of extracellular matrix (ECM) or polymeric material.

17. A device as claimed in claim 16, wherein the ECM is formed from decellularised propria-submucosa of a ruminant forestomach.

18. A device as claimed in any preceding claim, wherein the fluid supply lumen of the removable conduit structure comprises a non-porous wall along at least a major part of the length of the structure.

19. A device as claimed in any preceding claim, wherein the fluid removal lumen of the removable conduit structure comprises a porous wall along a major part of the length of the structure.

20. A device as claimed in claim 19, comprising a truss defining at least a major portion of the fluid removal lumen of the removable conduit structure.

21. A device as claimed in claim 20, wherein the truss comprises two flexible elongate wall members wound such that they intersect each other periodically at a plurality of cross-over nodes.

22. A device as claimed in any preceding claim, wherein each elongate wall member is generally helical, and wherein the two wall members are oppositely wound.

23. A device as claimed in any preceding claim, wherein the truss forms a flexible tube having a round or oval cross-section.

24. A device as claimed in claim 21 or 22, comprising at least two flexible elongate bracing members, each bracing member being linked to the two elongate wall members at a plurality of the cross-over nodes.

25. A device as claimed in claim 24, wherein the bracing members extend generally longitudinally along a side of the channel.

26. A device as claimed in claim 25, wherein the bracing truss members are provided on opposite sides of the channel.

27. A device as claimed in any one of claims 24 to 26, wherein each bracing member is bonded to the two elongate wall members at the respective cross-over nodes.

28. A device as claimed in any one of claims 20 to 27, further comprising a securing truss member, wound to secure the truss of the fluid removal lumen to the fluid supply lumen.

29. A device as claimed in any one of claims 1 to 19, wherein the removable conduit structure comprises a silicone form.

30. A device as claimed in any preceding claim, wherein the fluid removal lumen has a cross-sectional area of at least 7 mm².

31. A device as claimed in claim 30, wherein the fluid removal lumen has a cross-sectional area of about 18 mm².

32. A device as claimed in any preceding claim, wherein the fluid removal lumen has an inlet end and an outlet end, and wherein the fluid supply lumen is configured to supply fluid to adjacent the inlet end of the fluid removal lumen.

33. A device as claimed in any preceding claim, wherein the fluid supply lumen and the fluid removal lumen are generally the same length and positioned adjacent each other.

34. A device as claimed in any one of claims 1 to 32, wherein the fluid supply lumen and the fluid removal lumen are colinear.

35. A device as claimed in claim 34, wherein the device forms a loop.

36. A device as claimed in claim 35, wherein the loop comprises two limbs of the conduit structure with abutted ends.

37. A device as claimed in any preceding claim, comprising a port in fluid communication with the fluid removal and/or fluid supply lumens and being connectable to a source of negative pressure or positive pressure.

38. A device as claimed in any preceding claim, wherein the treatment site is a region between surfaces or planes of muscle tissue, connective tissue and/or or skin tissue that have been separated during surgery or as a result of trauma, or a region within a layer of tissue.

39. A device as claimed in any preceding claim, wherein the sheath comprises a sealing end section free from apertures and having a tight fit with the underlying portion of the conduit structure.

40. A device as claimed in claim 39, wherein the sealing end section of the sheath extends over a portion of the conduit structure that comprises fluid impervious walls.

41. A device as claimed in claim 39 or 40, wherein the cross-sectional area of the sheath and the underlying conduit structure is reduced along at least a portion of the sealing section.

42. A device as claimed in claim 41, wherein the cross-sectional area of the sheath and the underlying conduit structure is tapered along at least a portion of the sealing section.

43. A device for implantation at a treatment site in the body of a patient for the delivery of fluid to and/or removal of fluid from the treatment site; the device comprising:
a conduit structure defining a fluid supply and/or removal lumen; and
a bioresorbable sheath surrounding a portion of the removable conduit structure, and comprising a plurality of apertures sized and positioned to enable fluid communication between the treatment site and the conduit structure while preventing blockages in the device.

44. A device as claimed in claim 43, wherein the apertures in the sheath each have an area of between about 0.2 mm² to about 0.8 mm².

45. A device as claimed in claim 43 or 44, wherein the sheath comprises a sealing end section free from apertures and having a tight fit with the underlying portion of the conduit structure.

46. A device as claimed in claim 45, wherein the sealing end section of the sheath extends over a portion of the conduit structure that comprises fluid impervious walls.

47. A device as claimed in claim 45 or 46, wherein the cross-sectional area of the sheath and the underlying conduit structure is reduced along at least a portion of the sealing section.

48. A device as claimed in claim 47, wherein the cross-sectional area of the sheath and the underlying conduit structure is tapered along at least a portion of the sealing section.

49. A device as claimed in any one of claims 43 to 48, comprising a port in fluid communication with the lumen(s) of the conduit structure.

50. A device as claimed in any one of claims 43 to 49, wherein the conduit structure comprises a removable component configured for removal from the treatment site upon completion of treatment.

51. A device for implantation at a treatment site in the body of a patient for the delivery of fluid to and/or removal of fluid from the treatment site; the device comprising:
a conduit structure defining a fluid supply lumen and a porous fluid removal lumen, one end of the fluid supply lumen being in fluid communication with a first end of the fluid removal lumen;
a bioresorbable sheath surrounding a portion of the removable conduit structure; and
a port in fluid communication with the fluid supply lumen and/or the fluid removal lumen(s).

52. A device as claimed in claim 51, comprising a dual lumen port, with a first lumen of the port in fluid communication with the fluid supply lumen and a second lumen of the port in fluid communication with the fluid removal lumen.

53. A device as claimed in claim 51 or 52, wherein a portion of the conduit structure defining the fluid supply lumen is integrally formed with a portion of the conduit structure defining the fluid removal lumen.

54. A device as claimed in any one of claims 51 to 53, wherein the fluid supply lumen and fluid removal lumen are co-axial.

55. A device as claimed in any one of claims 51 to 53, wherein the fluid supply lumen and fluid removal lumen are substantially parallel.

56. A device as claimed in any one of claims 51 to 55, wherein the port is configured for connection with one or more external components.

57. A device as claimed in any one of claims 51 to 52, wherein the sheath comprises a multiplicity of apertures to facilitate fluid transfer across the sheath, each aperture having an area of between about 0.2 mm² to about 0.8 mm².

58. A device as claimed in claim 43 or 44, wherein the sheath comprises a sealing end section free from apertures and having a tight fit with the underlying portion of the conduit structure.

59. A device as claimed in any one of claims 43 to 49, wherein the conduit structure comprises a removable component configured for removal from the treatment site upon completion of treatment.

60. A system for draining fluid from a treatment site and delivering fluid to a treatment site in the body of a patient comprising:
(vi) a device as claimed in any one of claims 1 to 59;
(vii) a conduit releasably coupled to either a port of the device or to a fluid impermeable dressing;
(viii) a reservoir located external to the body of the patient and containing a treatment fluid, the reservoir in fluid communication with the fluid supply lumen;
(ix) a second reservoir located external to the body of the patient, the second reservoir in fluid communication with fluid removal lumen for receiving fluid from the device; and
(x) a source of pressure coupled to the conduit for delivering positive pressure or negative pressure to the device.

61. A system as claimed in claim 60, wherein the source of pressure is capable of delivering negative pressure to the device so that fluid is drained from the treatment site into the device and transferred through the conduit to the reservoir.

62. A system as claimed in claim 60, wherein the port of the device is positioned external to the patient's body.

63. A kit of parts for forming the device as claimed in any one of claims 1 to 59, comprising a conduit structure defining a fluid removal lumen, and a bioresorbable sheath defining a passage for receipt of the conduit structure.

64. A kit of parts as claimed in claim 63, wherein the bioresorbable sheath is generally tubular having two open ends.
